(19) United States
(12) Patent Application Publication
(10) Pub. No.: US 2010/0215710 A1
(43) Pub. Date: Aug. 26, 2010

(54) Beta-2 Adrenergic Receptor Agonists and Antagonists and Modulation of Wound Healing

(75) Inventors: Roslyn Rivkah Isseroff, Sacramento, CA (US); Christine E. Pullar, Sacramento, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of the University of California

(21) Appl. No.: 11/886,363

(22) PCT Filed: Apr. 10, 2006
(86) PCT No.: PCT/US06/13415

§ 371 Date: Feb. 26, 2008

Related U.S. Application Data
(60) Provisional application No. 60/669,839, filed on Apr. 8, 2005.

(51) Int. Cl. A61K 31/5377; C07D 417/12; C07C 237/32; A61K 31/166; C07C 215/56; A61K 31/138; C07D 209/88; A61K 31/403; C07D 215/20; A61K 31/4704; C07C 215/64; C07C 311/32; A61K 31/18; C07C 215/60; A61K 31/137; A61K 9/00; A61P 17/02; A61P 27/02

(52) U.S. Cl. 424/423; 544/134; 514/236.2; 564/165; 514/620; 564/428; 514/652; 548/444; 514/411; 546/158; 514/312; 564/443; 514/650; 564/99; 514/605; 514/653

(57) ABSTRACT

Methods for increasing rate of healing of wounds in epithelial tissues by administration of beta-2 adrenergic receptor antagonists to target patients are provided. Methods for decreasing cell growth around implanted devices and methods for decreasing wound contraction by administration of beta-2 adrenergic receptor agonists are also provided. Pharmaceutical compositions and kits including beta-2 adrenergic receptor agonists and antagonists are described, as are devices coated with beta-2 adrenergic receptor agonists.

### CROSS-REFERENCE TO RELATED APPLICATIONS

This application is a non-provisional utility patent application claiming priority to and benefit of the following prior provisional patent application: U.S. Ser. No. 60/669,839, filed Apr. 8, 2005, entitled “BETA-2 ADRENERGIC RECEPTOR AGONISTS AND ANTAGONISTS AND MODULATION OF WOUND HEALING” by Isseroff and Pullar, which is incorporated herein by reference in its entirety for all purposes.

### STATEMENT AS TO RIGHTS TO INVENTIONS MADE UNDER FEDERALLY SPONSORED RESEARCH AND DEVELOPMENT

This invention was made with government support under Grant. Nos. AR44518 and AR048827 from the National Institutes of Health. The government may have certain rights to this invention.

### FIELD OF THE INVENTION

The present invention is in the field of wound healing. The invention relates to methods for modulating wound healing, wound contraction, and/or epithelialization by modulating beta-2 adrenergic receptor activity using agonists and antagonists. The invention also relates to compositions, kits, and devices comprising beta-2 adrenergic receptor agonists and antagonists for modulating wound healing, wound contraction, and/or epithelialization.

### BACKGROUND OF THE INVENTION

Impaired wound healing is a growing clinical problem, most evident in the remarkable numbers of chronic wounds in our aging population: 6.5 million have chronic skin ulcers caused by pressure, venous stasis, or diabetes mellitus (Cupp and Bloom (2002) Gene therapy, electroporation, and the future of wound-healing therapies. Facial Plast Surg 18:53-57), costing the health care system a staggering $9 billion annually (Ashcroft et al. (2003) Estrogen modulates cutaneous wound healing by downregulating macrophage migration inhibitory factor. J Clin Invest 111:1309-1318; Ruckley (1997) Angiology 48:67-69; Phillips (1994) J Invest Dermatol 102:38 S-41S; Kantor and Margolis (2003) Semin Cutan Med Surg 22:212-221; and Margolis (2004) Int J Low Extrem Wounds 3:4-6). There is thus a need for treatments that can improve healing (e.g., re-epithelialization) of such chronic wounds.

Epithelialization of surfaces where such cell growth is not desirable, for example, around an implanted medical device, is a related problem. Such cell growth around a device such as an indwelling catheter or stent can decrease the effectiveness of the device and necessitate its frequent replacement, at increased cost and increased risk to the patient. Means of decreasing such unwanted epithelial cell growth are thus likewise desirable.

Among other aspects, the present invention provides methods and compositions that can improve healing of wounds, e.g., chronic wounds, or decrease undesirable cell growth around implanted devices. A complete understanding of the invention will be obtained upon review of the following.

### SUMMARY OF THE INVENTION

Modulation of beta-2 adrenergic receptor activity by administration of agonists and antagonists can influence epithelial cell growth. The present invention provides methods for increasing rate of wound healing by administration of β2-AR antagonists. The invention also provides methods for decreasing cell growth around implanted devices and for decreasing wound contraction by administration of β2-AR agonists. Novel pharmaceutical compositions for topical administration of β2-AR agonists and antagonists are also described, as are kits for administering such agonists and antagonists and devices coated with such agonists.

One general class of embodiments provides a pharmaceutical composition that includes a beta-2 adrenergic receptor antagonist. The composition is formulated for topical delivery of the antagonist to a tissue or organ other than an eye.

In one class of embodiments, the composition is formulated for topical delivery of the antagonist to skin. For example, the composition can comprise an ointment, cream, or lotion. One class of embodiments provides a dressing comprising the composition. The dressing can be impregnated with the composition, or at least one surface of the dressing can be coated with the composition.

Exemplary antagonists include, but are not limited to, timolol, labetalol, dilevelol, propanolol, carvedilol, nadolol, carteolol, penbutolol, sotalol, ICI 118,551, and butoxamine. In some embodiments, the antagonist has a K_{d} for a beta-3 adrenergic receptor that is about 100 or more times greater than a K_{d} of the antagonist for a beta-2 adrenergic receptor. In some embodiments, the antagonist is substantially free of activity as a beta-3 adrenergic receptor agonist.

Another general class of embodiments provides a pharmaceutical composition comprising a beta-2 adrenergic receptor agonist. The composition is formulated for topical delivery of the agonist to a tissue or organ, which tissue or organ is other than an eye or a tissue or organ comprising a respiratory tract.

Essentially all of the features noted for the embodiments above apply to this class of embodiments as well, as relevant. For example, in one class of embodiments, the composition is formulated for topical delivery of the agonist to skin. Thus, the composition optionally comprises an ointment, cream, or lotion. One class of embodiments provides a dressing comprising the composition. The dressing can be impregnated with the composition, or at least one surface of the dressing can be coated with the composition.

Exemplary agonists include, but are not limited to, isoproterenol, L-dobutamine, salbutamol, albuterol, terbutaline, bambuterol, fenoterol, formoterol, reproterol, salmeterol, tolubuterol, metaproterenol, pirbuterol, and ritrodine.

Yet another general class of embodiments provides a kit that includes a pharmaceutical composition comprising a beta-2 adrenergic receptor agonist or antagonist and instructions for administering the composition to a patient comprising or at risk for comprising a wound in an epithelial tissue, packaged in one or more containers (e.g., a flexible tube containing the composition).

The composition is optionally formulated for topical delivery of the agonist or antagonist. For example, the composition can be formulated for topical delivery of the agonist or antagonist to skin.

Essentially all of the features noted for the embodiments above apply to this class of embodiments as well, as relevant, e.g., with respect to type of agonist or antagonist. Thus, for example, the composition can comprise an ointment, cream, or lotion. The kit optionally includes a dressing comprising the composition, wherein the dressing is impregnated with the composition or wherein at least one surface of the dressing is coated with the composition.

In one aspect, the present invention provides methods for increasing the rate of repair of wounds in epithelial tissues, e.g., in humans. The methods involve administration of β2-AR antagonists to stimulate wound repair.

One general class of embodiments provides methods for increasing a rate of wound healing in a target patient. In the methods, the target patient is identified by identifying a person comprising or at risk for comprising a wound in an epithelial tissue, and an effective amount of a beta-2 adrenergic receptor antagonist is topically administered to the target patient.

In one class of embodiments, the wound comprises a chronic skin wound, e.g., a venous stasis ulcer, a diabetic foot ulcer, a neuropathic ulcer, or a decubitus ulcer. In another class of embodiments, the wound results from surgical wound dehiscence. The methods can also be applied to other types of wounds. For example, the wound can comprise a burn, cut, incision, laceration, ulceration, abrasion, or essentially any other wound in an epithelial tissue.

The methods can be applied to repair of wounds in essentially any epithelial tissue, including, but not limited to, skin, a genitourinary epithelium, a gastrointestinal epithelium, a pulmonary epithelium, or a corneal epithelium.

As noted, the antagonist is administered topically. For example, the antagonist can be topically administered by application of an ointment, cream, lotion, gel, suspension, spray, or the like comprising the antagonist to the wound. As another example, the antagonist can be topically administered by application of a dressing comprising the antagonist to the wound, e.g., a dressing impregnated with the antagonist or having at least one surface coated with the antagonist, e.g., a pad or self-adhesive bandage. As yet another example, the antagonist can be topically administered by introduction of a foam (e.g., a biologically inert or pharmaceutically acceptable foam) or other carrier comprising the antagonist to an epithelial-lined cavity comprising the wound, e.g., an oral, vaginal, or bladder cavity.

Treatment is optionally prophylactic; e.g., the antagonist can be administered to a patient at risk for comprising a wound. Thus, in some embodiments, the antagonist is administered prior to creation of the wound or at the time of wounding. More typically, however, the antagonist is administered after the wound is created, e.g., after the patient presents to a physician for treatment of a chronic wound.

Essentially all of the features noted for the embodiments above apply to this class of embodiments as well, as relevant, e.g., with respect to type of antagonist.

Another general class of embodiments also provides methods for increasing a rate of wound healing in a target patient. In the methods, the target patient is identified by identifying a person comprising or at risk for comprising a wound in an epithelial tissue, and an effective amount of a beta-2 adrenergic receptor antagonist is administered to the target patient. In this general class of embodiments, the wound is other than a burn. Exemplary wounds to which the methods can be applied include, but are not limited to, a chronic skin wound (e.g., a venous stasis ulcer, a diabetic foot ulcer, a neuropathic ulcer, or a decubitus ulcer), a wound resulting from surgical wound dehiscence, a cut, an incision, a laceration, an ulcer, an abrasion, or essentially any wound in an epithelial tissue that is other than a burn.

The antagonist can be administered systemically, locally, and/or topically. For example, the antagonist can be administered systemically, e.g., orally or intravenously. As another example, the antagonist can be administered topically, e.g., by application of an ointment, cream, lotion, gel, suspension, spray, dressing, foam, or the like comprising the antagonist to the wound. As yet another example, the antagonist can be administered by injecting the antagonist directly into tissue underlying or immediately adjacent to the wound.

Essentially all of the features noted for the methods above apply to this class of embodiments as well, as relevant, for example, with respect to epithelial tissue, time of administration, antagonist used, and the like.

The methods of the invention can increase the rate of wound healing by a statistically significant amount. Yet another general class of embodiments thus provides methods for increasing a rate of wound healing in a target patient. In the methods, the target patient is identified by identifying a person comprising or at risk for comprising a wound in an epithelial tissue, and an effective amount of a beta-2 adrenergic receptor antagonist is administered to the target patient. In this class of embodiments, the rate of wound healing in the target patient treated with the antagonist is at least about 10% greater than in a corresponding untreated individual. For example, the rate of wound healing in the target patient treated with the antagonist can be at least about 15% greater or at least about 20% greater than in a corresponding untreated individual.

Essentially all of the features noted for the methods above apply to this class of embodiments as well, as relevant, for example, with respect to type of wound, epithelial tissue, administration, and/or antagonist.

Administration of a beta-2 adrenergic receptor antagonist can improve healing of burns. Thus, yet another general class of embodiments provides methods for increasing a rate of wound healing in a target patient. In the methods, the target patient is identified by identifying a person comprising or at risk for comprising a wound in an epithelial tissue, wherein the wound is a burn, and an effective amount of a beta-2 adrenergic receptor antagonist is administered to the target patient. In one aspect, the patient does not display hypermetabolic syndrome (also known as a hypermetabolic response). In one aspect, the burn covers less than about 40% of the patient's total body surface area, optionally less than about 30% or less than about 20% of the patient's total body surface area.

Essentially all of the features noted for the methods above apply to this class of embodiments as well, as relevant, for example, with respect to type of epithelial tissue, administration, and/or antagonist.

In another aspect, the invention provides methods for decreasing cell growth around a device implanted in a target organism. In the methods, the target organism is identified by identifying an organism having or expected to have a device implanted in the organism, and an effective amount of a beta-2 adrenergic receptor agonist is administered to the target organism (e.g., a human).

The agonist is optionally administered systemically, e.g., orally or intravenously, or locally. For example, in one class of embodiments, the agonist is administered by coating the device with the agonist prior to implantation of the device in the organism.

The methods can be used to reduce (e.g., prevent) epithelialization of essentially any implantable device whose function is impaired by such cell growth, including, but not limited to, a stent or catheter. The device can be implanted, for example, in a blood vessel, urinary tract, airway, gastrointestinal tract, bile duct, or the like.

Essentially all of the features noted for the embodiments above apply to this class of embodiments as well, as relevant, e.g., with respect to type of agonist.

Coated devices form another feature of the invention. Thus, one general class of embodiments provides a coated device for implantation in an organism (e.g., a human). The coated device includes a device and a coating on a surface of the device. The coating includes a beta-2 adrenergic receptor agonist.

Essentially all of the features noted for the embodiments above apply to this class of embodiments as well, as relevant, e.g., with respect to agonists used. For example, the device can comprise a stent, a catheter, or essentially any other implantable device whose function can be impaired by epithelialization of the device.

Yet another aspect of the invention provides methods for decreasing wound contraction by administration of a beta2-AR agonist. Thus, one general class of embodiments provides methods for decreasing wound contraction in a target patient. In the methods, the target patient is identified by identifying a person comprising or at risk for comprising a wound in an epithelial tissue, and an effective amount of a beta-2 adrenergic receptor agonist is administered to the target patient.

Essentially all of the features noted for the embodiments above apply to this class of embodiments as well, as relevant, e.g., with respect to type of agonist, mode of administration, time of administration, and the like. For example, the wound can comprise, e.g., a burn or a surgical incision, and the agonist can be administered systemically, locally, and/or topically.

### BRIEF DESCRIPTION OF THE DRAWINGS

FIG. 1 Panel A depicts photographs of scratch wounds 40 hours after wounding, for a control wound (left) and a wound treated with 1 μM clenbuterol (right). Panel B presents a graph showing percent wound healing in control (◯) and clenbuterol (□) treated wounds. Panel C presents a graph showing speed of single cell migration in control, clenbuterol, OA, and OA/clenbuterol treated keratinocytes.

FIG. 2 Panels A and B present graphs of keratinocyte number over time in control cells (◯), cells treated with 1 μM β-AR agonist (□), cells pre-treated with 10 nM OA for 30 minutes prior to the addition of OA alone (X), or cells pre-treated with 10 nM OA for 30 minutes prior to the addition of both OA and β-AR agonist ( [symbol] ).

FIG. 3 Panel A presents a graph of the number of wounds healed over time, for control and β2-AR agonist treated wounds. Panel B depicts photographs of sections from control (left) and β2-AR agonist treated (right) wounds. Panel C presents a graph showing percent re-epithelialization for control (◯) and 10 μM β-AR agonist (□) treated wounds.

FIG. 4 Panel A depicts immunoblots probed with an anti-ERK antibody (bottom) or anti-phospho-ERK antibody (top) after wounding in β-AR agonist treated and untreated wound discs. Panel B presents a graph showing phospho-ERK levels at various times after wounding in β-AR agonist treated and untreated wound discs.

FIG. 5 Panel A depicts photographs of scratch wounds 16 hours after wounding, for a control wound (left) and a wound treated with 10 nM ICI 118,551 (right). Panel B presents a graph showing percent wound healing in control (◯) and ICI 118,551 (□) treated wounds.

FIG. 6 Panel A presents a graph showing speed of single cell migration in control and timolol treated keratinocytes. Panel B presents a graph showing distance traveled by single cells in control and timolol treated keratinocytes.

FIG. 7 Panel A depicts immunoblots probed with an anti-ERK antibody (bottom) or anti-phospho-ERK antibody (top), for antagonist treated keratinocytes. Panel B presents a graph showing average percent increase in phosphorylated ERK over time for antagonist treated keratinocytes.

FIG. 8 presents a graph representing directionality of cell migration in control and antagonist treated keratinocytes.

FIG. 9 presents a graph of cell number over time for control and antagonist treated keratinocytes.

FIG. 10 Panel A presents a graph of the number of wounds healed over time, for control and β2-AR antagonist treated wounds. Panel B depicts photographs of sections from control (left) and β2-AR antagonist treated (right) wounds. Panel C presents a graph showing percent re-epithelialization for control (◯) and antagonist (□) treated wounds. Panel D depicts photographs of biopsies cultured for an additional four days in the presence or absence of additional serum and a graph showing percent re-epithelialization.

FIG. 11 Panel A schematically illustrates the catecholamine biosynthesis cascade. Panel B depicts immunoblots probed with an anti-PNMT antibody (top) or an anti-TH antibody (bottom) in lysates from three different keratinocyte strains, PC12 cells, and dermal fibroblasts.

FIG. 12 Panel A presents a graph showing percent wound healing in control and β2-AR antagonist treated wounds. Panel B depicts photographs of scratch wounds 0 and 20 hours after wounding, for control (left) and antagonist treated (right) wounds.

FIG. 13 Panel A depicts immunoblots probed with an anti-phospho-ERK antibody (top) and anti-ERK antibody (bottom) for antagonist treated CECs. Panel B presents a graph showing average percent increase in phosphorylated ERK over time for antagonist treated CECs.

FIG. 14 Panel A presents a graph showing distance traveled by single control and treated corneal epithelial cells. Panel B presents a graph showing speed of single cell migration, for control and treated corneal epithelial cells.

FIG. 15 Panel A presents a graph showing trajectory speed and displacement speed for control, agonist treated, and antagonist treated CECs. Panel B presents a graph representing directionality of cell migration for control, agonist treated, and antagonist treated CECs. Panel C present graphs of cell trajectories for control, agonist treated, and antagonist treated CECs.

FIG. 16 presents a graph showing cell number as a function of time for control, agonist treated, and antagonist treated CECs.

FIG. 17 Panel A presents a graph showing percent wound healing over time for control, agonist treated, and antagonist treated wounds. Panel B depicts photographs of control, agonist treated, and antagonist treated wounds. Panel C depicts photographs of control, agonist treated, and antagonist treated wounds.

FIG. 18 depicts immunoblots probed with an anti-PNMT antibody (top) or an anti-TH antibody (bottom).

FIG. 19 Panel A depicts photographs of fluorescein stained corneal epithelial wounds from β2-AR +/+ and −/− mice treated with BSS (control), isoproterenol (agonist), or timolol (antagonist), over time after wounding. Panel B presents a graph showing rate of wound healing in control, agonist, and antagonist treated β2-AR +/+ and −/− mice.

FIG. 20 depicts photographs of control (Panel A), agonist treated (Panel B), and antagonist treated (Panel C) human skin burn wounds 10 days after wounding.

### DEFINITIONS

Unless defined otherwise, all technical and scientific terms used herein have the same meaning as commonly understood by one of ordinary skill in the art to which the invention pertains. The following definitions supplement those in the art and are directed to the current application and are not to be imputed to any related or unrelated case, e.g., to any commonly owned patent or application. Although any methods and materials similar or equivalent to those described herein can be used in the practice for testing of the present invention, the preferred materials and methods are described herein. Accordingly, the terminology used herein is for the purpose of describing particular embodiments only, and is not intended to be limiting.

As used in this specification and the appended claims, the singular forms “a,” “an” and “the” include plural referents unless the context clearly dictates otherwise. Thus, for example, reference to “a receptor” includes a plurality of receptors; reference to “a cell” includes mixtures of cells, and the like.

The term “about” as used herein indicates the value of a given quantity varies by +/−10% of the value, or optionally +/−5% of the value, or in some embodiments, by +/−1% of the value so described.

The term “systemic” refers to distribution throughout the body, as opposed to “local”. A compound (e.g., an agonist or antagonist) administered “systemically” (orally or intravenously, for example) is distributed to the entire body, e.g., by traveling through the bloodstream.

The term “topical” refers to administration or delivery of a compound (e.g., an agonist or antagonist) by application of the compound to a surface of a body part. For example, a compound can be topically administered by applying it to skin, a mucus membrane, or another body surface. Topical administration can result, e.g., in either local or systemic delivery of a compound.

An “agonist” is a compound (e.g., an endogenous substance or a drug) that can bind to and activate a receptor, thereby initiating a response (e.g., a physiological or pharmacological response) characteristic of that receptor. For example, for the beta-2 adrenergic receptor, an increase in the intracellular concentration of cyclic AMP can be assayed (see, e.g., Chen et al. (2002) Beta-adrenergic receptor activation inhibits keratinocyte migration via a cyclic adenosine monophosphate-independent mechanism. J Invest Dermatol 119:1261-1268). Agonists can be, e.g., full agonists or partial agonists.

An “antagonist” is a compound (e.g., a drug) that can bind to a receptor and prevent an agonist from binding to and activating that receptor. Typically, binding of an antagonist to a receptor forms a complex which does not give rise to any response, as if the receptor were unoccupied. Alternatively, the antagonist can be a partial agonist.

It is worth noting that certain compounds can be classified as both an agonist and an antagonist. For example, a “mixed agonist-antagonist” (also called a “partial agonist”) is a compound which possesses affinity for a receptor, but which, unlike a full agonist, will elicit only a small degree of the response characteristic of that receptor, even if a high proportion of receptors are occupied by the compound. Such occupancy of the receptors by the partial agonist can prevent binding of a full agonist (e.g., an endogenous agonist) to the receptor.

An “effective amount” of an agonist or antagonist refers to an amount of the agonist or antagonist that produces a specified effect, e.g., that increases a rate of wound healing, decreases cell growth around a device, or decreases wound contraction.

A person “at risk for comprising a wound” has a higher probability of developing a wound than does the general population. Examples include a diabetic patient expected to comprise diabetic ulcers or a person anticipating surgery.

A “pharmaceutical composition” is a composition that can be used on or in the body to prevent, alleviate, treat, or cure a disease, disorder, or other condition, such as a wound, in a human or animal.

A variety of additional terms are defined or otherwise characterized herein.

### DETAILED DESCRIPTION

Beta-adrenergic receptors (β-ARs) are expressed on a wide variety of tissues and are recognized as pivotal functional regulators of the cardiac, pulmonary, vascular, endocrine and central nervous systems. There are at least three subtypes of β-ARs: beta-1, beta-2, and beta-3. (See, e.g., the Online Mendelian Inheritance in Man entries for ADRB1, ADRB2, and ADRB3 on the world wide web at ncbi.nlm.nih.gov/Omim/.) Although expression of beta-adrenergic receptors in human skin was noted over 30 years ago (Tseraidis and Bavykina (1972) [Adrenergic innervation of normal human skin]. Vestn Dermatol Venerol 46:40-45), only recently has their functional significance in this tissue begun to be recognized. The β2-AR subtype is optionally the only subtype of β-ARs expressed on the membranes of the major cell types in skin: keratinocytes, fibroblasts, and melanocytes (Schallreuter et al. (1993) Increased in vitro expression of beta 2-adrenoceptors in differentiating lesional keratinocytes of vitiligo patients. Arch Dermatol Res 285:216-220; Steinkraus et al. (1992) Binding of beta-adrenergic receptors in human skin. J Invest Dermatol 98:475-480; Steinkraus et al. (1996) Autoradiographic mapping of beta-adrenoceptors in human skin. Arch Dermatol Res 288:549-553; McSwigan et al. (1981) Down syndrome fibroblasts are hyperresponsive to beta-adrenergic stimulation. Proc Natl Acad Sci USA 78:7670-7673; and Gillbro et al. (2004) Autocrine catecholamine biosynthesis and the beta-adrenoceptor signal promote pigmentation in human epidermal melanocytes. J Invest Dermatol 123:346-353).

Activation of the beta-2 adrenergic receptor (β2-AR or beta2-AR) by agonists can decrease keratinocyte migration and proliferation, thereby delaying healing of cutaneous wounds. Conversely, β2-AR antagonists can enhance epithelial cell growth and migration and thus stimulate wound repair.

The present invention thus provides methods for increasing rate of wound healing by administration of β2-AR antagonists. The invention also provides methods for decreasing cell growth around implanted devices and for decreasing wound contraction by administration of β2-AR agonists. Novel pharmaceutical compositions for topical administration of β2-AR agonists and antagonists are also described, as are kits for administering such agonists and antagonists.

### Methods for Stimulating Wound Healing

A wound in an epithelial tissue typically disrupts the continuity of the epithelial layer. For example, a wound in the skin typically disrupts (e.g., completely removes a section of) the epidermis, and, depending on the depth of the wound, can also remove part of the dermis. Healing of a wound in an epithelial tissue generally involves migration and/or proliferation of cells surrounding the wound, and the wound is typically considered to be healed when the wound is re-epithelialized, e.g., covered by at least one layer of cells.

In one aspect, the present invention provides methods for increasing the rate of repair of wounds in epithelial tissues, e.g., in humans. The methods involve administration of β2-AR antagonists to stimulate wound repair (i.e., re-epithelialization of the area), e.g., by stimulating migration and/or proliferation of epithelial cells (e.g., of keratinocytes for repair of a wound in the skin).

A first general class of embodiments provides methods for increasing a rate of wound healing in a target patient. In the methods, the target patient is by identifying a person comprising or at risk for comprising a wound in an epithelial tissue, and an effective amount of a beta-2 adrenergic receptor antagonist is topically administered to the target patient.

In one class of embodiments, the wound is in skin. The methods can be particularly useful for stimulating healing of chronic, non-healing skin wounds. Thus, in one class of embodiments, the wound comprises a chronic skin wound, e.g., a venous stasis ulcer, a diabetic foot ulcer, a neuropathic ulcer, or a decubitus ulcer. Other exemplary chronic wounds for which the methods can be used include, but are not limited to, other chronic ulcers such as immune-mediated (e.g., rheumatoid arthritis) ulcers, radiotherapy-induced ulcers, and ulcers resulting from vasculitis, arteriolar obstruction or occlusion, pyoderma gangrenosum, thalessemai, and other dermatologic diseases that result in non-healing wounds. In a related class of embodiments, the wound results from surgical wound dehiscence.

The methods can also be applied to other types of wounds. For example, the wound can comprise a burn, cut, incision, laceration, ulceration, abrasion, or essentially any other wound in an epithelial tissue.

Similarly, the methods can be applied to repair of wounds in essentially any epithelial tissue, including, but not limited to, skin, a genitourinary epithelium, a gastrointestinal epithelium, a pulmonary epithelium, or a corneal epithelium.

In one aspect, the antagonist is administered topically. For example, the antagonist can be topically administered by application of an ointment, cream, lotion, gel, suspension, spray, or the like comprising the antagonist to the wound. As another example, the antagonist can be topically administered by application of a dressing comprising the antagonist to the wound, e.g., a dressing impregnated with the antagonist or having at least one surface coated with the antagonist, e.g., a pad or self-adhesive bandage.

As yet another example, the antagonist can be topically administered by application of a transdermal device. Either “passive” or “active” transdermal devices can be employed for administration of one or more compositions of the invention, the selection of which will depend in part upon the location for application of the device (e.g., at or proximal to the site of epithelial damage for local administration of, for example, rapidly metabolized compositions, or distal to the site for systemic composition administration). Examples of passive transdermal devices include reservoir-type patches (e.g., in which the composition is provided within a walled reservoir having a permeable surface) and matrix-type patches (in which the composition is dispersed within a polymeric composition). Active transdermal devices include, but are not limited to, devices employing iontophoresis (e.g., a low voltage electrical current), electroporation (e.g., short electrical pulses of higher voltage), sonophoresis (e.g., low frequency ultrasonic energy), or thermal energy for delivery of the composition. Typically, passive-type transdermal devices would be utilized for application at a current site of epithelial damage, since additional mechanisms for overcoming the epithelial barrier provided by active-type transdermal devices is not necessary. For a review of various transdermal technologies, see Ghosh, Pfister and Yum Eds. (1997) Transdermal and Topical Drug Delivery Systems (CRC Press, London); Potts and Guy (Eds.) (1997) Transdermal Drug Delivery (Marcel Dekker, New York); and Potts and Cleary (1995) Transdermal drug delivery: useful paradigms. J Drug Targ. 3:247-251.

As yet another example, the antagonist can be topically administered by introduction of a foam (e.g., a biologically inert or pharmaceutically acceptable foam) or other carrier comprising the antagonist to an epithelial-lined cavity comprising the wound, e.g., an oral, vaginal, or bladder cavity.

It will be evident that various means of administration can be combined, for the same or different antagonists. Thus, for example, the antagonist can be administered both topically and orally or topically and by injection, simultaneously or sequentially, as indicated by the nature and severity of the wound to be treated.

Treatment is optionally prophylactic; e.g., the antagonist can be administered to a patient at risk for comprising a wound. Thus, in some embodiments, the antagonist is administered prior to creation of the wound or at the time of wounding. More typically, however, the antagonist is administered after the wound is created, e.g., after the patient presents to a physician for treatment of a chronic wound.

A large number of antagonists are known in the art and can be adapted to the practice of the present invention. Exemplary antagonists are described in greater detail below in the section entitled “Agonists and Antagonists.”

A second general class of embodiments also provides methods for increasing a rate of wound healing in a target patient. In the methods, the target patient is identified by identifying a person comprising or at risk for comprising a wound in an epithelial tissue, and an effective amount of a beta-2 adrenergic receptor antagonist is administered to the target patient. In this general class of embodiments, the wound is other than a burn. Exemplary wounds to which the methods can be applied include, but are not limited to, a chronic skin wound (e.g., a venous stasis ulcer, a diabetic foot ulcer, a neuropathic ulcer, a decubitus ulcer, an immune-mediated ulcer, a radiotherapy-induced ulcer, or an ulcer resulting from vasculitis, arteriolar obstruction or occlusion, pyoderma gangrenosum, thalessemai, or another dermatologic disease that results in non-healing wounds), a wound resulting from surgical wound dehiscence, a cut, an incision, a laceration, an ulcer, an abrasion, or essentially any other wound (other than a burn) in an epithelial tissue.

The methods can be applied to repair of wounds in essentially any epithelial tissue, including, but not limited to, skin, a genitourinary epithelium, a gastrointestinal epithelium, a pulmonary epithelium, or a corneal epithelium. Optionally, the epithelial tissue is other than an epithelial tissue comprising an eye.

The antagonist can be administered systemically, locally, and/or topically. For example, the antagonist can be administered systemically, e.g., orally or intravenously. As another example, the antagonist can be administered topically, e.g., by application of an ointment, cream, lotion, gel, suspension, spray, dressing, transdermal device, foam, or the like comprising the antagonist to the wound. As yet another example, the antagonist can be administered locally or intralesionally by injecting the antagonist directly into tissue underlying or immediately adjacent to the wound. For example, for a skin wound, the antagonist can be administered by injecting it subcutaneously or intradermally at or near the site of the skin wound.

Essentially all of the features noted for the methods above apply to this class of embodiments as well, as relevant, for example, with respect to time of administration, antagonist used, and the like.

The methods of the invention can increase the rate of wound healing by a statistically significant amount. A third general class of embodiments thus provides methods for increasing a rate of wound healing in a target patient. In the methods, the target patient is identified by identifying a person comprising or at risk for comprising a wound in an epithelial tissue, and an effective amount of a beta-2 adrenergic receptor antagonist is administered to the target patient. In this class of embodiments, the rate of wound healing in the target patient treated with the antagonist is at least about 10% greater than in a corresponding untreated individual (e.g., at least about 15% greater or at least about 20% greater).

Essentially all of the features noted for the methods above apply to this class of embodiments as well, as relevant, for example, with respect to type of wound, epithelial tissue, administration, and/or antagonist.

As noted herein, administration of a beta-2 adrenergic receptor antagonist can improve healing of burns. Thus, another general class of embodiments provides methods for increasing a rate of wound healing in a target patient. In the methods, the target patient is identified by identifying a person comprising or at risk for comprising a wound in an epithelial tissue, wherein the wound is a burn, and an effective amount of a beta-2 adrenergic receptor antagonist is administered to the target patient.

In one aspect, the patient does not display hypermetabolic syndrome or a hypermetabolic response. Hypermetabolic syndrome, described in the literature, can occur with burns covering greater than 40% of the patient's total body surface area. In one aspect, the burn covers less than about 80% of the patient's total body surface area, e.g., less than about 70%, 60%, or 50% of the patient's total body surface area. In one class of embodiments, the burn covers less than about 40% of the patient's total body surface area, optionally less than about 35%, less than about 30%, less than about 20%, or even less than about 10% or less than about 5% of the patient's total body surface area. It will be evident that the area covered by the burn can be continuous or discontinuous. The patient may or may not display a hypermetabolic response.

Essentially all of the features noted for the methods above apply to this class of embodiments as well, as relevant, for example, with respect to type of epithelial tissue, administration, and/or antagonist. Thus, for example, the epithelial tissue can comprise skin. The antagonist is optionally administered systemically, topically, by application of an ointment, cream, lotion, gel, suspension, spray, or dressing, and/or by injection. Exemplary antagonists include those listed herein.

Another general class of embodiments provides methods for increasing a rate of wound healing in a target organism. In the methods, the target organism is identified by identifying an organism comprising or at risk for comprising a wound in an epithelial tissue, and an effective amount of a beta-2 adrenergic receptor antagonist is administered to the target organism. In this general class of embodiments, the wound is other than a burn, and the epithelial tissue is other than a corneal epithelium. The organism can be, e.g., a human, a non-human mammal, a mammal, or a vertebrate.

Essentially all of the features noted for the methods above apply to this class of embodiments as well, as relevant. For example, it is worth noting that the antagonist can be administered systemically, locally, and/or topically.

In a related aspect, the invention provides methods in which a beta-2 AR antagonist is administered to a target patient comprising a wound in an epithelial tissue to increase wound contraction. Administration of the antagonist is optionally continued to stimulate re-epithelialization and complete healing of the wound, as described above. The methods can be useful, e.g., in initial stages of treatment of an acute wound or treatment of surgical dehiscence, to decrease the area of the wound.

### Methods for Decreasing Cell Growth and Wound Contraction

In another aspect, the invention provides methods for decreasing the rate of cell growth (e.g., epithelial cell growth) around a device introduced into an organism by administration of a beta2-AR agonist. The methods can thus reduce, or optionally prevent, cell growth around an implanted device (including, e.g., cell growth around exterior and/or interior surfaces of the device), reducing or preventing epithelialization which can otherwise encapsulate or clog the device or otherwise interfere with its performance.

Thus, one general class of embodiments provides methods for decreasing cell growth around a device implanted in a target organism. In the methods, the target organism is identified by identifying an organism having or expected to have a device implanted in the organism, and an effective amount of a beta-2 adrenergic receptor agonist is administered to the target organism. The target organism can be a human, a non-human mammal, a vertebrate, or the like.

The agonist is optionally administered systemically, e.g., orally or intravenously, or locally. For example, in one class of embodiments; the agonist is administered by coating the device with the agonist prior to implantation of the device in the organism. The agonist is optionally administered transdermally. It will be evident that various means of administration can be combined, for the same or different agonists. Thus, for example, the agonist can be administered both orally and by coating the device with the agonist, simultaneously or sequentially as needed. Treatment with the agonist may begin before, at the time of, or after implantation of the device in the organism.

The methods can be used to reduce (e.g., prevent) epithelialization of essentially any implantable device whose function is impaired by such cell growth, including, but not limited to, a stent (e.g., a coronary, peripheral, or GI stent), catheter (e.g., an indwelling catheter), anastomosis device, birth control occlusion device, breast implant, dental implant, focal epilepsy treatment device, heart valve repair, implantable biosensor, implanted drug infusion tube, intravitreal drug delivery device, nerve regeneration conduit, neuro aneurysm treatment device, pacemaker and electrostimulation leads, pain management device, prostate cancer treatment device, spinal repair device, vascular graft, or vena cava filter. The methods can also include reducing cell growth around a fistula or the like. The device can be implanted, for example, in a blood vessel, urinary tract, airway, gastrointestinal tract, bile duct, or the like. Cell growth around the device is optionally inhibited or prevented for one month or more, six months or more, twelve months or more, eighteen months or more, or twenty-four months or more. Duration of the inhibition can depend, e.g., on the half-life of the agonist coating the implanted device, duration of time for which the agonist is systemically administered, or the like.

A large number of agonists are known in the art and can be adapted to the practice of the present invention. Exemplary agonists are described in greater detail below in the section entitled “Agonists and Antagonists.” Coating compositions which can be adapted to the practice of the present invention (e.g., to provide sustained release of the agonist) are also known in the art and are described in greater detail below in the section entitled “Compositions, Kits, and Devices.”

Yet another aspect of the invention provides methods for decreasing wound contraction by administration of a beta2-AR agonist. Undesirable wound contraction can occur, e.g., as a result of burns or trauma, resulting in both cosmetic and functional problems ranging from minimal cosmetic scarring to major body deformation and loss of joint mobility. The ability to decrease wound contraction can reduce scarring and deformation and enhance joint mobility, e.g., in cosmetic surgery, burn, and trauma patients. For example, the methods can be used to minimize or prevent wound contraction in wounds that overlie functionally sensitive areas, such as joints, or near orifices (e.g., eyes, mouth, etc.), where contraction would decrease function of the joint or use of the orifice. See also Pullar and Isseroff (2005a) “Beta 2-adrenergic receptor activation delays dermal fibroblast-mediated contraction of collagen gels via a cAMP-dependent mechanism” Wound Repair Regen 13:405-11.

Thus, one general class of embodiments provides methods for decreasing wound contraction in a target patient. In the methods, the target patient is identified by identifying a person comprising or at risk for comprising a wound in an epithelial tissue, and an effective amount of a beta-2 adrenergic receptor agonist is administered to the target patient.

The methods can be applied to wounds in essentially any epithelial tissue, including, but not limited to, skin, a genitourinary epithelium, a gastrointestinal epithelium, a pulmonary epithelium, or a corneal epithelium. The wound can comprise, e.g., a burn or a surgical incision.

The agonist is optionally administered by injecting the agonist directly into tissue underlying or immediately adjacent to the wound. For example, for a skin wound, the agonist can be administered by injecting it subcutaneously or intradermally at or near the site of the skin wound. The agonist can be administered systemically, e.g., orally or intravenously.

In one aspect, the agonist is administered topically. For example, the agonist can be topically administered by application of an ointment, cream, lotion, gel, suspension, spray, or the like comprising the agonist to the wound. As another example, the agonist can be topically administered by application of a dressing comprising the agonist to the wound, e.g., a dressing impregnated with the agonist or having at least one surface coated with the agonist, e.g., a pad or self-adhesive bandage. As yet another example, the agonist can be topically administered by introduction of a foam (e.g., a biologically inert or pharmaceutically acceptable foam) comprising the agonist to an epithelial-lined cavity comprising the wound, e.g., an oral, vaginal, or bladder cavity. As yet another example, the agonist can be topically administered by application of a transdermal device.

It will be evident that various means of administration can be combined, for the same or different agonists. Thus, for example, the agonist can be administered both locally and systemically, simultaneously or sequentially, as indicated by the nature and severity of the wound to be treated.

Treatment is optionally prophylactic; e.g., the agonist can be administered to a patient at risk for comprising a wound. Thus, in some embodiments, the antagonist is administered prior to creation of the wound (e.g., prior to surgery) or at the time of wounding. In other embodiments, the agonist is administered after the wound is created.

A large number of agonists are known in the art and can be adapted to the practice of the present invention. Exemplary agonists are described in greater detail below in the section entitled “Agonists and Antagonists.”

### Compositions, Kits, and Devices

The methods of the invention optionally include novel topical therapy with well-known and characterized drugs, beta-AR agonists and antagonists, e.g., to modulate wound healing and/or contraction. The invention thus includes novel compositions for topical application of beta-AR agonists and antagonists. Kits and devices related to the methods are also provided.

One general class of embodiments provides a pharmaceutical composition that includes a beta-2 adrenergic receptor antagonist. The composition is formulated for topical delivery of the antagonist to a tissue or organ other than an eye. Typically, the composition includes an effective amount of the antagonist and/or is formulated for delivery of an effective amount of the antagonist to the tissue or organ. For example, the composition can be formulated for delivery depending on the partitioning of the drug from the vehicle into the tissue, to ultimately deliver an effective amount of antagonist into the tissue or organ.

In one preferred class of embodiments, the composition is formulated for topical delivery of the antagonist to skin. For example, the composition can comprise an ointment (e.g., an occlusive or petrolatum-based ointment), cream, lotion, gel, spray, foam, or the like, e.g., in which the antagonist is suspended, dissolved, or dispersed. Many suitable bases for such ointments, creams, lotions, gels, etc. are known in the art and can be adapted to the practice of the present invention. At least one component of the composition is optionally insoluble in water and/or hydrophobic; for example, the composition optionally includes an oil (e.g., a suspension of an oil in water), petrolatum, a lipid, or the like.

One class of embodiments provides a dressing comprising the composition. The dressing can be impregnated with the composition, or at least one surface of the dressing can be coated with the composition. The composition is optionally formulated for slow, controlled release of the antagonist. The dressing can be a bulky dressing, a pad, a bandage, a self-adhesive bandage, or other suitable biocompatible dressing. A related class of embodiments provides a transdermal device comprising the composition.

As noted above, a variety of antagonists are known in the art and can be adapted to the practice of the present invention. Exemplary antagonists are described in greater detail below in the section entitled “Agonists and Antagonists.”

Another general class of embodiments provides a pharmaceutical composition comprising a beta-2 adrenergic receptor agonist. The composition is formulated for topical delivery of the agonist to a tissue or organ, which tissue or organ is other than an eye or a tissue or organ comprising a respiratory tract. Typically, the composition includes an effective amount of the agonist and/or is formulated for delivery of an effective amount of the agonist to the tissue or organ.

Essentially all of the features noted for the embodiments above apply to this class of embodiments as well, as relevant. For example, in a preferred class of embodiments, the composition is formulated for topical delivery of the agonist to skin. Thus, the composition optionally comprises an ointment, cream, lotion, gel, spray, foam, or the like. Similarly, a transdermal device comprising the composition or a dressing comprising the composition is a feature of the invention.

As noted above, a variety of agonists are known in the art and can be adapted to the practice of the present invention. Exemplary agonists are described in greater detail below in the section entitled “Agonists and Antagonists.”

Yet another general class of embodiments provides a kit that includes a pharmaceutical composition comprising a beta-2 adrenergic receptor agonist or antagonist and instructions for administering the composition to a patient comprising or at risk for comprising a wound in an epithelial tissue, packaged in one or more containers.

The composition is optionally formulated for systemic (e.g., oral or intravenous) delivery of the agonist or antagonist. Alternatively, the composition can be formulated for local delivery of the agonist or antagonist. In one preferred class of embodiments, the composition is formulated for topical delivery of the agonist or antagonist. For example, the composition can be formulated for topical delivery of the agonist or antagonist to skin.

Essentially all of the features noted for the embodiments above apply to this class of embodiments as well, as relevant. Thus, for example, the composition can comprise an ointment, cream, lotion, gel, spray, foam, or the like. The kit optionally includes a dressing comprising the composition, wherein the dressing is impregnated with the composition or wherein at least one surface of the dressing is coated with the composition, or a transdermal device comprising the composition.

The one or more containers optionally include a flexible tube containing the composition (e.g., in embodiments in which the composition is formulated as an ointment, cream, or lotion). Similarly, the container(s) can comprise a bottle, vial, spray or aerosol can, or other suitable container.

As mentioned previously, many agonists and antagonists are known in the art and can be adapted to the practice of the present invention. Exemplary agonists and antagonists are described in greater detail below in the section entitled “Agonists and Antagonists.”

Yet another general class of embodiments provides a coated device for implantation in an organism (e.g., a human). The coated device includes a device and a coating on a surface of the device. The coating includes a beta-2 adrenergic receptor agonist, e.g., an effective amount of the agonist.

Essentially all of the features noted for the embodiments above apply to this class of embodiments as well, as relevant. For example, the device can comprise a stent (e.g., a coronary, peripheral, or GI stent), a catheter, or essentially any other implantable device whose function can be impaired by epithelialization of the device, including, but not limited to, an anastomosis device, birth control occlusion device, breast implant, dental implant, focal epilepsy treatment device, heart valve repair, implantable biosensor, implanted drug infusion tube, intravitreal drug delivery device, nerve regeneration conduit, neuro aneurysm treatment device, pacemaker and electrostimulation leads, pain management device, prostate cancer treatment device, spinal repair device, vascular graft, or vena cava filter.

The coating on the surface of the device is optionally formulated for slow, controlled release of the agonist, e.g., over a period of one month or more, six months or more, twelve months or more, eighteen months or more, or twenty-four months or more. Exemplary agonists are described, e.g., in the section below entitled “Agonists and Antagonists.”

Device coatings that can be adapted to the practice of the present invention, e.g., for sustained release of a beta-2 AR agonist, are known in the art. Examples include, but are not limited to, nanofilm coatings (e.g., including porous hydroxyapatite), porous nanostructured elemental silicon coatings, phosphorylcholine coatings, and polymeric coatings (including, e.g., thermoresponsive polymers, hydrogels, N-isopropylacrylamide-based thermoresponsive co-polymers, polyacrylic, methacrylate, hydrocarbon-based elastomeric polymers (e.g., a 50:50 polymer mix of polyethylenevinylacetate and polybutylmethcrylate), and/or a poly(organo)-phosphazene polymer). See, e.g., Kavanagh et al. (2004) Local drug delivery in restenosis injury: Thermoresponsive co-polymers as potential drug delivery systems. Pharmacology & Therapeutics 102:1-15; Lewis and Stratford (2002) Phosphorylcholine-coated stents. J Long Term Eff Med Implants. 12:231-50; and Montdargent and Letourneur (2000) Toward new biomaterials. Infect Control Hosp Epidemiol. 21:404-10. Exemplary coatings are commercially available, e.g., from pSivida Ltd and MIV Therapeutics (on the world wide web (www.) at psivida.com.au/ and mivtherapeutics.com/technology/drug_eluting_stents, respectively), among many others.

### Agonists and Antagonists

A wide variety of beta-2 AR agonists and antagonists are known and have been described in the scientific and patent literature, many of which are in clinical use for other conditions. Although a few exemplary agonists and antagonists are listed below, no attempt is made to identify all possible agonists and antagonists herein. Other suitable agonists and antagonists which can be adapted to the practice of the present invention can be readily identified by one of skill in the art.

An agonist or antagonist can be selective for the β2-AR, affecting substantially only the β2-AR, or it can be nonselective, affecting the β1 and β2 ARs, the β1, β2, and β3 ARs, or the like. It will be evident that selectivity is optionally a function of the concentration of the agonist or antagonist. For example, an antagonist can have a Kᵢ for the β2-AR that is 100-fold less than its Kᵢ for the β1-AR, in which example the antagonist is considered to be selective for the β2-AR over the β1-AR when used at a concentration relatively near its Kᵢ for the β2-AR (e.g., a concentration that is within about 10-fold of its Kᵢ for the β2-AR).

Exemplary nonselective β-AR agonists in clinical use include, but are not limited to, isoproterenol and dobutamine (e.g., L-dobutamine). Exemplary selective β2-AR agonists in clinical use include, but are not limited to, salbutamol, albuterol, terbutaline, bambuterol, fenoterol, formoterol, reproterol, salmeterol, tolubuterol, metaproterenol, pirbuterol, and ritrodine. Clenbuterol is another exemplary selective β2-AR agonist, although it is not currently in clinical use.

Exemplary nonselective β-AR antagonists in clinical use include, but are not limited to, timolol, labetalol, dilevelol, propanolol, carvedilol, nadolol, carteolol, penbutolol, and sotalol. Exemplary selective β2-AR antagonists include, but are not limited to, ICI 118,551 and butoxamine.

As noted, an antagonist can be selective or nonselective for the β2-AR. Similarly, in certain embodiments, the antagonist has a greater affinity for the β2-AR than for the β3-AR. Thus, in one aspect, the antagonist has a K_{d} for a beta-3 adrenergic receptor that is about 100 or more times greater than a K_{d} of the antagonist for a beta-2 adrenergic receptor. In one aspect, the antagonist is substantially free of activity as a beta-3 adrenergic receptor agonist, e.g., has no detectable activity as a β3-AR agonist. For example, antagonists for use in the invention optionally, exclude CGP 12177.

Choice of agonist or antagonist for a particular application can be influenced, for example, by factors such as the half-life of the compound, its selectivity, potential side effects, preferred mode of administration, potency, and clinical information about a given patient (e.g., any known pre-existing conditions that might be exacerbated by administration of an agonist or antagonist, potential drug interactions, or the like). For example, ritrodine is typically suitable for intravenous injection and not for use as an inhalant. Nadolol has a long half-life (on the order of 24 hours), and potentially has lower central nervous system side effects due to low lipid solubility.

The amount of agonist or antagonist to be administered in the treatment of wounds or reduction of cell growth according to the present invention can depend, e.g., on the nature, severity, and extent of the wound to be treated, the potency of the compound, the patient's weight, the patient's clinical history and response to the agonist or antagonist, and the discretion of the attending physician. Appropriate dosage can readily be determined by one of skill in the art. For systemic administration, the dose is optionally between about 0.01 and 30 mg per kg of body weight per day. This dose can optionally be subdivided into 2, 3, 4 or more administrations throughout the day.

The agonist or antagonist is suitably administered to the patient at one time or over a series of treatments. For repeated administrations over several days or longer, depending on the condition, the treatment is optionally sustained until a desired result occurs; for example, until a wound is healed. Similarly, treatment can be maintained as required, e.g., to maintain suppression of dell growth around an implanted device. The progress of the therapy can be monitored by conventional techniques and assays.

A pharmaceutical composition of the present invention for topical administration, e.g., an ointment, cream, lotion, foam, or gel (e.g., an aqueous gel), or, in general, a solution or suspension of the agonist or antagonist, typically contains from 0.01 to 10% w/v (weight/volume, where 1 g/100 ml is equivalent to 1%) of the agonist or antagonist, preferably from 0.1 to 5% w/v, e.g., mixed with customary excipients or dissolved in an appropriate vehicle for topical application. Exemplary compositions formulated for topical application to skin have been described above. Compositions formulated for topical administration to the eye include, e.g., aqueous gels and aqueous drops in buffered salt solutions, and ocular ointments including the antagonist or agonist.

In a pharmaceutical composition of the present invention for oral, sublingual, subcutaneous, intramuscular, intravenous, transdermal, or local administration, for example, the agonists or antagonists can be administered in unit forms of administration, either as such, for example in lyophilized form, or mixed with conventional pharmaceutical carriers. Appropriate unit forms of administration include oral forms such as tablets, which may be divisible, gelatin capsules, powders, granules and solutions or suspensions to be taken orally, sublingual and buccal forms of administration, subcutaneous, intramuscular or intravenous forms of administration, and local forms of administration.

When a solid composition is prepared in the form of tablets, the main active ingredient is optionally mixed with a pharmaceutical vehicle such as gelatin, starch, lactose, magnesium stearate, talcum, gum arabic or the like. The tablets can be coated with sucrose or other appropriate substances, or can be treated so as to have a prolonged or delayed activity and so as to release a predetermined amount of active principle continuously. A preparation in the form of gelatin capsules can be obtained by mixing the active ingredient with a diluent and pouring the resulting mixture into, soft or hard gelatin capsules. A preparation in the form of a syrup or elixir optionally contains the active ingredient together with a sweetener, antiseptic, flavoring and/or appropriate color. Water-dispersible powders or granules can contain the active ingredient mixed with dispersants, wetting agents or suspending agents, as well as with sweeteners or taste correctors. Suppositories (e.g., for vaginal or rectal administration) can be prepared with binders melting at the appropriate (e.g., vaginal or rectal) temperature. Parenteral administration is typically effected using aqueous suspensions, saline solutions or injectable sterile solutions containing pharmacologically compatible dispersants and/or wetting agents. The agonist or antagonist is optionally encapsulated in liposomes or otherwise formulated for prolonged or delayed release, e.g., whether for topical, local, and/or systemic administration.

### EXAMPLES

It is understood that the examples and embodiments described herein are for illustrative purposes only and that various modifications or changes in light thereof will be suggested to persons skilled in the art and are to be included within the spirit and purview of this application and scope of the ended claims. Accordingly, the following examples are offered to illustrate, but not to limit, the claimed invention.

### Example 1

### β2-Adrenergic Receptor Activation Delays Wound Healing

The following sets forth a series of experiments that demonstrate use of β2-AR agonists to decrease the rate of re-epithelialization in cell culture and in human skin explants.

Keratinocytes, which are thought to solely express the β2-adrenergic receptor (β2-AR) subtype of β-ARs, migrate directionally into the wound bed to initiate re-epithelialization, typically necessary for wound closure and restoration of barrier function. β2-AR activation affects keratinocyte migration, proliferation, cytoskeletal conformation, phospho-ERK localization, wound re-epithelialization, and wound-induced ERK phosphorylation. β2-AR activation is anti-motogenic and anti-mitogenic, with both mechanisms being PP2A-dependent. Additionally, β2-AR activation dramatically alters the conformation of the actin cytoskeleton and prevents the localization of phospho-ERK to the lamellipodial edge. Finally, β2-AR activation delays re-epithelialization and leads to a decrease in wound-induced epidermal ERK phosphorylation in human skin wounds.

As noted, the β2-AR subtype is optionally the only subtype of β-ARs expressed on the membranes of the major cell types in skin: keratinocytes, fibroblasts, and melanocytes. Cutaneous keratinocytes also actively synthesize catecholamine ligands for these receptors (Schallreuter et al. (1995) Catecholamines in human keratinocyte differentiation. J Invest Dermatol 104:953:957 and Schallreuter (1997) Epidermal adrenergic signal transduction as part of the neuronal network in the human epidermis. J Investig Dermatol Symp Proc 2:37-40), thus creating a self-contained hormonal mediator network. Keratinocyte-generated catecholamines have recently been demonstrated to regulate skin melanogenesis, thus providing one of the first clues as to the homeostatic regulatory function of this cutaneous paracrine signaling network (Gillbro et al., supra). Interestingly, aberrations in either keratinocyte β2-AR function or density have also been associated with cutaneous disease. Keratinocytes derived from patients with atopic eczema display a point mutation in, the β2-AR gene and a low β2-AR density (Schallreuter (1997) supra). In psoriasis, keratinocytes within the psoriatic lesions demonstrate a low cAMP response to β2-AR activation (Eedy et al. (1990) Beta-adrenergic stimulation of cyclic AMP is defective in cultured dermal fibroblasts of psoriatic subjects. Br J Dermatol 122:477-483). These findings point to a role for the cutaneous β2-AR network in maintaining epidermal function and integrity. This example provides data that supports a role for the β2-AR network in regulating cutaneous wound repair as well.

Cutaneous wound healing is a complex and well-orchestrated biological process requiring the coordinated migration and proliferation of both keratinocytes and fibroblasts, as well as other cell types. Wounding the epidermis generates cytokines, growth factors, proteases and the synthesis of extracellular matrix components, all of which can regulate the processes of keratinocyte migration and proliferation; generally essential for re-epithelialization (Martin (1997) Wound healing—aiming for perfect skin regeneration. Science 276:75-81 and Singer and Clark (1999) Cutaneous wound healing. N Engl J Med 341:738-746). The first clues to a biological function for β2-AR in wound repair came from an early study demonstrating that β2-AR agonists delay skin wound healing in the newt limb (Donaldson and Mahan (1984) Influence of catecholamines on epidermal cell migration during wound closure in adult newts. Comp Biochem Physiol C 78:267-270). Subsequent studies in other epithelia, however, have yielded conflicting results. For example, β-AR antagonists have been reported to either delay (Haruta et al. (1997) Corneal epithelial deficiency induced by the use of beta-blocker eye drops. Eur J Opthalmol 7:334-339 and Liu et al. (1990) Beta adrenoceptors and regenerating corneal epithelium. J Ocul Pharmacol 6:101-112) or enhance (Reidy et al. (1994) Effect of topical beta blockers on corneal epithelial wound healing in the rabbit. Br J Opthalmol 78:377-380) corneal epithelial wound healing.

This example focuses on the effects of β-AR agonists on the constituent cells of human skin. β-AR agonists decrease keratinocyte migration in vitro (Chen et al. (2002) Beta-adrenergic receptor activation inhibits keratinocyte migration via a cyclic adenosine monophosphate-independent mechanism. J Invest Dermatol 119:1261-1268 and Pullar et al. (2003) PP2A activation by beta2-adrenergic receptor agonists: novel regulatory mechanism of keratinocyte migration. J Biol Chem 278:22555-22562). Unlike other cell types previously studied, where β-AR agonist binding activates ERK (Zou et al. (2001) Isoproterenol activates extracellular signal-regulated protein kinases in cardiomyocytes through calcineurin. Circulation 104:102-108; Jordan et al. (2001) Oligomerization of opioid receptors with beta 2-adrenergic receptors: a role in trafficking and mitogen-activated protein kinase activation. Proc Natl Acad Sci USA 98:343-348; Crespo et al. (1995) Dual effect of beta-adrenergic receptors on mitogen-activated protein kinase. Evidence for a beta gamma-dependent activation and a G alpha s-cAMP-mediated inhibition. J Biol Chem 270:25259-25265; Ahn et al. (1999) Src-mediated tyrosine phosphorylation of dynamin is required for beta2-adrenergic receptor internalization and mitogen-activated protein kinase signaling. J Biol Chem 274:1185-1188; Bogoyevitch et al. (1996) Adrenergic receptor stimulation of the mitogen-activated protein kinase cascade and cardiac hypertrophy. Biochem J 314:115-121; Maudsley et al. (2000) The beta(2)-adrenergic receptor mediates extracellular signal-regulated kinase activation via assembly of a multi-receptor complex with the epidermal growth factor receptor. J Biol Chem 275:9572-9580; and Schmitt and Stork (2000) beta 2-adrenergic receptor activates extracellular signal-regulated kinases (ERKs) via the small G protein rap1 and the serine/threonine kinase B-Raf. J Biol Chem 275:25342-25350), in keratinocytes β-AR agonists reduce ERK phosphorylation, notably in a cAMP-independent (Chen et al. supra) and PP2A-dependent manner (Pullar et al. supra). Since ERK phosphorylation is activated upon mechanical injury of keratinocytes (Turchi et al. (2002) Dynamic Characterization of the Molecular Events During In Vitro Epidermal Wound Healing. J Invest Dermatol 119:56-63) and is also required for keratinocyte migration (Zeigler et al. (1999) Role of ERK and INK pathways in regulating cell motility and matrix metalloproteinase 9 production in growth factor-stimulated human epidermal keratinocytes. J Cell Physiol 180:271-284) and proliferation (Sharma et al. (2003) p38 and ERK1/2 coordinate cellular migration and proliferation in epithelial wound healing: evidence of cross-talk activation between MAP kinase cascades. J Biol Chem 278:21989-21997), these findings suggest (without intending to be limited to any particular mechanism) that β2-adrenergic signaling could impact wound repair by modulating ERK phosphorylation, thus regulating the dual critical processes of keratinocyte migration and proliferation.

This example examines the role of β2-AR signaling in human skin wound re-epithelialization. β2-AR activation in human keratinocytes impairs their migration in vitro and their ability to repair a scratch wound in culture. β-AR agonist-induced alterations in the keratinocyte actin cytoskeleton, focal adhesion morphology and lamellipodial localization of phospho-ERK are observed, which optionally contribute to the impaired migratory phenotype. Finally, β2-AR activation induces inhibition of wound re-epithelialization in organ-cultured human skin. β2-AR activation significantly delays wound re-epithelialization and notably decreases the wound-induced phosphorylation of ERK in the peri-wound epidermis. Without intending to be limited to any particular mechanism, these findings indicate that the anti-motogenic and anti-mitogenic effects of β2-AR activation observed in keratinocytes in vitro can underlie the impairment of the re-epithelialization process observed in wounded human skin and point to a previously unrecognized novel role for the adrenergic hormonal network as a regulator of the wound healing process.

### Materials and Methods

Keratinocyte Growth

Human keratinocytes were isolated from neonatal foreskins as reported previously (Isseroff et al. (1987) Conversion of linoleic acid into arachidonic acid by cultured murine and human keratinocytes. J Lipid Res 28:1342-1349) and cultured using a modification of the method of Rheinwald and Green (Rheinwald and Green (1975) Serial cultivation of strains of human epidermal keratinocytes: the formation of keratinizing colonies from single cells. Cell 6:331-343). Cells were grown in keratinocyte growth medium (KGM) (Epilife, 0.06 mM Ca²⁺), supplemented with human keratinocyte growth supplement (0.2 ng/ml EGF, 5 μg/ml insulin, 5 μg/ml transferrin, 0.18 μg/ml hydrocortisone and 0.2% bovine pituitary extract) (Cascade Biologics, Inc., Portland, Oreg.) and antibiotics (100 U/ml penicillin, 100 μg/ml streptomycin, and 0.25 μg/ml amphotericin) (Gemini Bio-Products, Inc., Calabasas, Calif.) at 37° C. in a humidified atmosphere of 5% CO₂. Cell strains isolated from at least two different foreskins were used and experiments were performed with sub-cultured cells between passages 4-7.

Scratch Assay

Cells were grown to confluence on cover-slips (Fisher Scientific, Pittsburgh, Pa.) coated with 60 μg/ml Collagen I (Vitrogen 100, Collagen Corp., Palo Alto, Calif.). Cells were either untreated (control) or treated with clenbuterol (1 μM) (Calbiochem, San Diego, Calif.) at time 0. A sterile pipette tip was used to scratch a 1 mm-wide wound along the center of the dish and a demarcated area of the wound was photographed on an inverted Nikon Diaphot microscope at the time of wounding (time 0) up to wound healing (Haas et al. (1990) Low-energy helium-neon laser irradiation increases the motility of cultured human keratinocytes. J Invest Dermatol 94:822-826). The area of the wound was determined using NIH Image 1.6 and the percent wound healing calculated by dividing the area of the wound at time X by the area of the wound at time 0 and multiplying by 100. Significance was taken as p<0.01, using Student's t test (unpaired) to compare the mean percent healing of the control and β-AR agonist-treated % wounds. NIH Image is a public domain image processing and analysis program for the Macintosh, developed at the U.S. National Institutes of Health and available on the Internet at rsb.info.nih.gov/nih-image/.

Single Cell Migration Assay

All single cell migration assays were performed on cells plated on glass cover-slips inserted into 35 mm plastic dishes (MatTek Corp., Ashland, Mass.) that had been coated for 1 hour at 37° C. with 60 μg/ml collagen L Cells were plated onto the collagen-coated glass cover-slips at a density of 250 cells/mm² for 3-6 hours at 37° C. Cells were either untreated (control), pre-treated with 10 nM okadaic acid (OA) (10 nM) for 45 minutes prior to the addition of OA (10 nM) and clenbuterol (1 μM) (OA/Clen) or treated with either OA or clenbuterol (1 μM) alone at time 0. The dishes were inserted into metal plates, maintained at 37° C., on inverted Nikon Diaphot microscopes to monitor single cell migration. Time-lapse images of the cell migratory response were digitally captured every 10 minutes over a one-hour period by Q-Imaging Retiga-EX cameras (Burnaby, BC, Canada) controlled by a custom automation written in Improvision Open Lab software (Lexington, Mass.) on a Macintosh G4. After each cell's center of mass was tracked using the Open Lab software, migration speed and distance were calculated and imported to Excel (Microsoft Corporation, Redmond, Wash.). Significance was taken as p<0.01, using Student's t test (unpaired) to compare the means of the control and β-AR agonist-treated cell populations.

Proliferation Assay

Keratinocytes were released from the tissue culture plate by treatment with 0.25% trypsin/EDTA (Gibco, Grand Island, N.Y.), resuspended in KGM and counted using a haemocytometer. Cells were either untreated or pre-treated with okadaic acid (OA) (10 nM) for 45 minutes prior to β-AR agonist (1 μM) addition. 5×10⁴ cells were plated per well in a 12 well plate in triplicate in the presence or absence of 1 μM β-AR agonist, 10 nM OA or both. Triplicate wells were harvested and counted on days 2, 4, 6, 8. The medium was changed every day. Significance was taken as p<0.01, using Student's t test (unpaired) to compare the means of the cell populations.

Immunofluorescent Staining

Sterile glass cover-slips (Fisher Scientific, Pittsburgh, Pa.) were transferred into 12 well dishes and collagen-coated with 60 μg/ml collagen I in KGM for 1 hour at 37° C. Cover-slips were washed three times with KGM and 3×10⁴ cells were added per well and allowed to attach overnight. Cells were untreated, treated with 1 μM β-AR agonist for 15 minutes, OA (10 nM) for 45 minutes, or pre-treated with OA (10 nM) for 30 minutes prior to the addition of 1 μM β-AR agonist for 15 minutes. All steps were performed at room temperature unless otherwise noted. Cover slips were washed twice in PBS and fixed for 10 minutes in 4% paraformaldehyde. Cover-slips were washed twice in PBS between each step. Cells were permeabilized for 5 minutes with 0.1% Triton-X-100/PBS, blocked with 5% goat serum/PBS for 20 minutes, primary monoclonal anti-vinculin antibody (Sigma, St. Louis, Mo.) or anti-phospho-ERK antibody (Cell Signaling Technology, Beverly, Mass.) were added drop-wise in 1% goat serum/PBS (1:100) and incubated for 1 hour at 37° C. A goat anti-mouse cy3 (Jackson labs, West Grove, Pa.) (1:100) antibody was then added in 1% goat serum/PBS for 1 hour at 37° C. Alexa 488-phalloidin (Molecular Probes, Eugene, Oreg.) (1:40) in PBS was added to the vinculin-stained cover-slips for 20 minutes. Finally, Prolong anti-fade reagent (Molecular Probes, Eugene, Oreg.) was used according to manufacturer's instructions to mount the cover-slips onto glass microscope slides. Slides were viewed on an inverted fluorescent Nikon Diaphot microscope using a 40× pan fluor objective. Images were captured using Q-imaging Retiga-EX cameras (Burnaby, BC, Canada) and pseudo-colored green for Alexa 488 Phalloidin staining (actin), red for Cy3 staining (vinculin), or visualized in grey scale for phospho-ERK staining using Improvision Openlab software (Lexington, Mass.).

Human Skin Wound Healing Assay

A wound healing model developed by Kratz (Kratz (1998) Modeling of wound healing processes in human skin using tissue culture. Microsc Res Tech 42:345-350) was adapted. Normal human skin was obtained from routine breast reductions or abdominoplasties under an approved exemption granted by the Internal Review Board at University of California, Davis. Under sterile conditions, excess subcutaneous fat was trimmed from 6″×3″ sections of skin prior to stretching and pinning onto sterile cork board. A 3 mm punch (Sklar Instruments, West Chester, Pa.) was used to make wounds in the epidermis and into the superficial dermis and the 3 mm discs of skin were excised using sterile scissors. 6 mm skin discs, with the 3 mm epidermal wound in the center, were excised using a 6 mm biopsy punch (SMS Inc., Columbia, Md.). The skin samples were immediately transferred to a 12 well dish (Costar, Cambridge, Mass.) and submerged in 2 ml of FM (Dulbecco's Modified Eagle's medium (DMEM) (Gibco, Grand Island, N.Y.) containing 10% fetal bovine serum (Tissue Culture Biologicals, Tulare, Calif.) and antibiotics (100 U/ml penicillin, 100 μg/ml streptomycin, and 0.25 μg/ml amphotericin)) (Gibco, Grand Island, N.Y.) in the presence or absence of 10 μM β-AR agonist. The 12 well dishes were incubated at 37° C. in a humidified atmosphere of 5% CO₂. The medium was changed every day. Three biopsies were fixed in 4% neutral buffered formaldehyde every day for 5 days. The formaldehyde-fixed biopsies were dehydrated through an ethanol-xylene series and embedded in paraffin. Cross-sections, 5 μM thick, taken from the center of the wound, were stained using the hematoxylin-eosin technique. Re-epithelialization was determined using light microscopy. A (+) score was given to a healed wound and a (−) score to any unhealed wounds. Specimens that were damaged in the histologic process or otherwise non interpretable were excluded from the study. Significance was taken as p<0.005, using the 2-tailed Fisher's exact test to compare the number of wounds healed versus unhealed in the absence or presence of β-AR agonist. Measuring the linear distance covered by new epithelium and dividing that by the linear distance between the original wound edges determined the percentage of re-epithelialization. The new epithelium was clearly differentiated from the epithelial wound margin by the presence of a fully stratified epithelium and fully formed stratum corneum in the latter. Significance was taken as p<0.05, using Student's t test (unpaired) to compare the means of the percent re-epithelialization of the control and β-AR agonist-treated wounds on each day.

Protein Extraction from Human Skin

To determine the phosphorylation state of ERK in wounded skin, the excised 3 mm epidermal discs were either pre-incubated in FM for 30 or 60 minutes in the presence or absence of 10 μM β-AR agonist prior to freezing or placed immediately into 500 μl of 1× Laemmli sample buffer (62.5 mM Tris-HCL, pH 6.8, 2% sodium dodecyl sulfate, 10% glycerol, 50 mM dithiothreitol) and snap frozen in liquid nitrogen, prior to storing at −80° C. Two 3 mm skin pieces were frozen per tube. Each tube was thawed for 10 minutes at 100° C. then centrifuged at 14,000 rpm for 10 minutes at 4° C. Protein concentrations were estimated by A₂₈₀, and equal microgram amounts were separated on 10% polyacrylamide Tris-HCl gels (Bio-Rad, Hercules, Calif.). Proteins were transferred to Immobilon membranes (Bio-Rad, Hercules, Calif.) and immunoblotted with either an anti-ERK antibody (#9102) or an anti-phospho-ERK antibody (#9101) (Cell Signaling Technology, Beverly, Mass.). The immunoblots were developed by enhanced chemiluminescence (ECL) according to the manufacturer's instructions (Amersham Pharmacia Biotech, Piscataway, N.J.). Densitometry was performed on scanned images using NIH Image 1.6. Significance was taken as p<0.01, using Student's t test (unpaired) to compare the means of the band intensities.

### Results

β2-AR Agonists Reduce Keratinocyte Scratch Wound Healing and OA Reverses the Agonist-Mediated Reduction in Migratory Speed

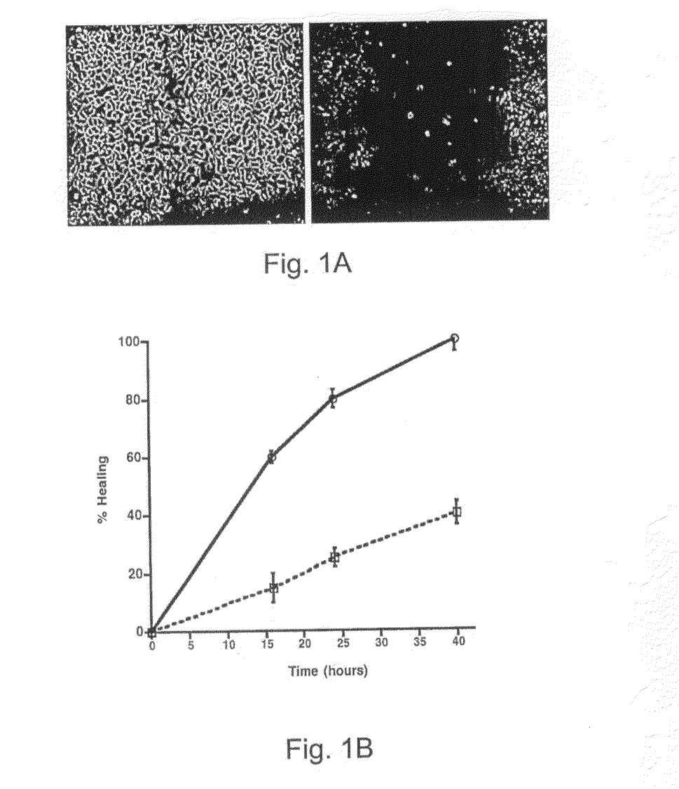

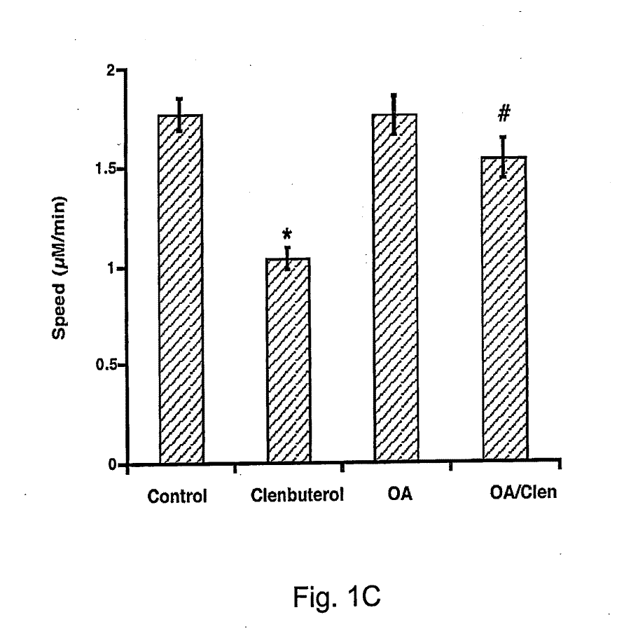

Keratinocytes were grown to confluence on collagen-coated glass cover-slips in the absence (control) or presence of 1 μM clenbuterol. Cultures were wounded as described, and a demarcated area of the wound was photographed at the time of wounding (time 0) and 16, 24, and 40 hours after wounding. Representative photographs of images at time 40 hours are shown in FIG. 1 Panel A. The percent wound healing was calculated and is represented graphically in FIG. 1 Panel B; control wounds are indicated by circles, clenbuterol treated by squares. The data shown are representative of three independent experiments from two separate cell strains. Values plotted are means+/−SEM (n=3).

Keratinocytes were plated onto collagen-coated glass cover-slips at a concentration of 250 cells/mm² in KGM for 3-6 hours at 37° C. The migration of each single cell was monitored over a one-hour period, and the results were analyzed in excel as described. The speed of migration (μM/min) is represented graphically in FIG. 1 Panel C, control n=57, clenbuterol n=57, OA n=60, OA-clenbuterol n=44. The data are representative of three independent experiments with at least two different keratinocyte strains. * P<0.01 between β-AR agonist and controls; #P<0.01 between OA-clenbuterol and clenbuterol alone.

The β-AR agonist clenbuterol significantly increases the time required for the scratch wounds, created within confluent monolayers of cultured human keratinocytes, to heal. Control wounds are completely healed by 40 hours, whereas clenbuterol-treated wounds are only 40% healed (FIG. 1 Panels A-B). To determine if the phosphatase PP2A modulates the clenbuterol-induced decrease in keratinocyte migration, the effect of okadaic acid (OA), a potent inhibitor of PP2A (Namboodiripad and Jennings (1996) Permeability characteristics of erythrocyte membrane to okadaic acid and calyculin A. Am J Physiol 270:C449-456; Millward et al. (1999) Regulation of protein kinase cascades by protein phosphatase 2A. Trends Biochem Sci 24:186-191; McMahon et al. (2001) Colony-stimulating factor-1 (CSF-1) receptor-mediated macrophage differentiation in myeloid cells: a role for tyrosine 559-dependent protein phosphatase 2A (PP2A) activity. Biochem J 358:431-436; and Fernandez et al. (2002) Okadaic acid, useful tool for studying cellular processes. Curr Med Chem 9:229-262), on migratory speed was examined. The addition of clenbuterol to the individually dispersed cells reduces keratinocyte migratory speed by 45% (*P<0.01, FIG. 1 Panel C). Although OA treatment alone has no effect on keratinocyte migration, it completely prevents the clenbuterol-induced reduction in speed traveled by the cells over the one-hour period (#P<0.01, FIG. 1 Panel C). These data demonstrate that the mechanism for the clenbuterol-mediated decrease in keratinocyte locomotory speed is PP2A-dependent.

β2-AR Agonists Reduce Keratinocyte Proliferation and OA Reverses the Agonist-Mediated Reduction in Proliferative Capacity

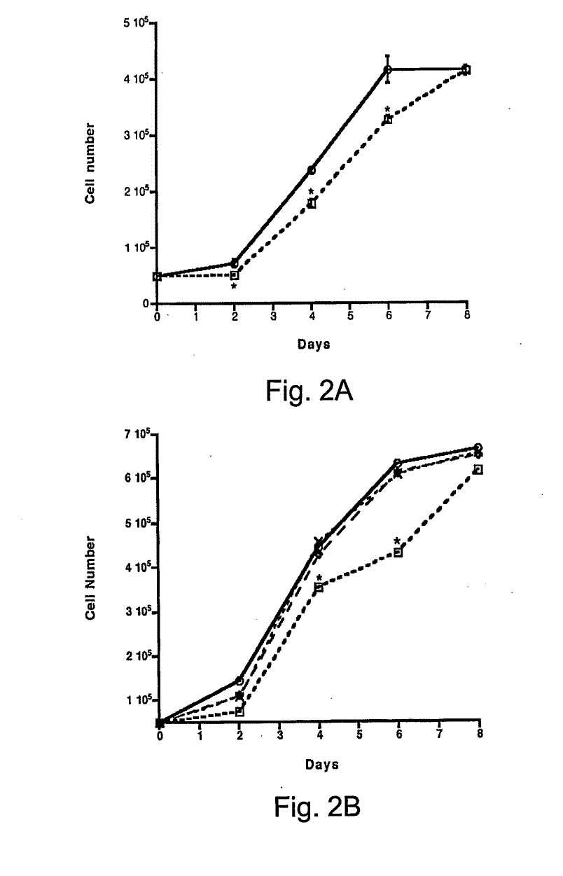

Since keratinocyte proliferation behind the migrating epithelial tongue is thought to be essential for effective re-epithelialization (Singer and Clark, supra), it was important to determine the effect of β2-AR activation on human keratinocyte proliferation. Therefore, human keratinocytes were grown in the presence or absence of β-AR agonist. β2-AR activation significantly decreases keratinocyte proliferation, with a maximum decrease of 21% at day 6 (FIG. 2 Panel A). The ability of OA to restore normal migration in β2-AR agonist-treated cells prompted investigation of whether it could also prevent the β2-AR-mediated decrease in keratinocyte proliferation. Indeed, OA alone has no effect on keratinocyte proliferation, but completely prevents the β2-AR-mediated decrease in proliferation (FIG. 2 Panel B). It therefore appears that the β2-AR-mediated decrease in proliferation is also mediated by a PP2A-dependent mechanism in human keratinocytes.

5×10⁴ dermal keratinocytes were plated per well in a 12 well plate in triplicate in the presence or absence of β-AR agonist (1 μM) (FIG. 2 Panel A; control represented by circles, 1 μM β-AR agonist by squares), or pre-treated with 10 nM OA for 30 minutes prior to the addition of OA alone or both OA and β-AR agonist, (FIG. 2 Panel B; OA represented by X's and OA/β-AR agonist by stars). Cells were harvested and counted on days 2, 4, 6, 8 as described. The data are representative of three independent experiments with at least two different keratinocyte strains. Values plotted are means+/−SEM. *P<0.01 between β-AR agonist and controls.

β2-AR Agonists Alter Keratinocyte Cytoskeletal Conformation and OA Prevents the β2-AR Mediated Changes in Cytoskeletal Conformation

Actin remodeling plays an important role in cell motility (Pantaloni et al. (2001) Mechanism of actin-based motility. Science 292:1502-1506) and proliferation (Ikeda et al. (2003) Aberrant actin cytoskeleton leads to accelerated proliferation of corneal epithelial cells in mice deficient for destrin (actin depolymerizing factor). Hum Mol Genet 12:1029-1037; Landriscina et al. (2000) Amlexanox reversibly inhibits cell migration and proliferation and induces the Src-dependent disassembly of actin stress fibers in vitro. J Biol Chem 275:32753-32762; Cuadros et al. (2000) The marine compound spisulosine, an inhibitor of cell proliferation, promotes the disassembly of actin stress fibers. Cancer Lett 152:23-29; Blakesley et al. (1998) Replacement of tyrosine 1251 in the carboxyl terminus of the insulin-like growth factor-I receptor disrupts the actin cytoskeleton and inhibits proliferation and anchorage-independent growth. J Biol Chem 273:18411-18422; Joneson et al. (1996) RAC regulation of actin polymerization and proliferation by a pathway distinct from Jun kinase. Science 274:1374-1376; and Sastrodihardjo et al. (1987) Possible involvement of reorganization of actin filaments, induced by tumor-promoting phorbol esters, in changes in colony shape and enhancement of proliferation of cultured epithelial cells. J Cell Physiol 132:49-56). Actin filaments terminate in focal adhesions (FAs), where several proteins, including vinculin mediate interactions with the actin cytoskeleton (Burridge and Fath (1989) Focal contacts: transmembrane links between the extracellular matrix and the cytoskeleton. Bioessays 10:104-108). FAs mediate the mechanical attachment of cells to the extra cellular matrix and therefore play an important role in anchorage-dependent mechanisms such as proliferation (Gilmore and Burridge (1996) Molecular mechanisms for focal adhesion assembly through regulation of protein-protein interactions. Structure 4:647-651). They also act as signaling centers, capable of regulating gene expression, cell growth and survival (Sastry and Burridge (2000) Focal adhesions: a nexus for intracellular signaling and cytoskeletal dynamics. Exp Cell Res 261:25-36). Additionally, small, nascent FAs have been associated with actively migrating cells (Beningo et al. (2001) Nascent focal adhesions are responsible for the generation of strong propulsive forces in migrating fibroblasts. J Cell Biol 153:881-888). Since β2-AR activation in keratinocytes is demonstrated to be anti-motogenic and anti-mitogenic, whether these effects may be mediated by alterations in the actin cytoskeleton was examined.

Sterile cover-slips were coated with collagen and cells plated as described. Cells were untreated, treated with 1 μM β-AR agonist for 15 minutes, treated with OA 10 nM for 45 minutes or pre-treated with OA 10 nM for 30 minutes prior to the addition of both OA 10 nM and 1 μM β-AR agonist for 15 minutes. Cells were fixed and stained for actin (green) and vinculin (red) as described. Three independent experiments from two separate cell strains were performed.

Cells plated in the absence of β-AR agonist are polarized and crescent shaped with a broad lamellipodium, characteristic of the migratory phenotype (Ridley et al. (2003) Cell migration: integrating signals from front to back. Science 302:1704-1709). Actin and vinculin staining reveals that the majority of the untreated keratinocytes have fine actin-rich lamellipodia containing multiple small linear vinculin-containing FAs. Pre-treating with β-AR agonist for 15 minutes (1 μM) markedly alters the keratinocyte morphology. Cells are now rounded with no apparent polarization. There is a marked increase in actin stress fibers localized at the internal borders of the cells and an increase in the number and size of vinculin-rich FAs, which are no longer localized to the lammellipodium.

To determine if the β2-AR agonist-mediated alteration in the cyto-architecture of actin stress fibers and vinculin-rich FAs was similarly mediated by PP2A, keratinocytes were pre-treated with the PP2A-specific inhibitor, OA, prior to exposure to β-AR agonist. OA treatment alone has no effect on the cytoskeletal conformation, with cells displaying a normal migratory phenotype. However, pre-treating keratinocytes with OA prior to adding β-AR agonists prevents the β2-AR-mediated change in cytoskeletal conformation. OA pre-treatment restores the migratory phenotype observed in untreated keratinocytes, confirming that the mechanism for the β2-AR-mediated alteration of cytoskeletal conformation is PP2A-dependent.

β2-AR Agonists Disrupt the Phosphorylation and Intracellular Localization of Phospho-ERK, while OA Preserves its Localization to the Leading Edge of the Keratinocyte Lamellipodium

ERK activation plays an important role in cell migration (Klemke et al. (1997) Regulation of cell motility by mitogen-activated protein kinase. J Cell Biol 137:481-492) and specifically keratinocyte migration (Zeigler et al., supra). To determine if β2-AR activation alters the cellular localization of phospho-ERK in keratinocytes, phospho-ERK was immunolocalized in the presence and absence of β-AR agonist.

Sterile cover-slips were coated with collagen and cells plated as described. Cells were untreated, treated with 1 μM β-AR agonist for 15 minutes, treated with OA 10 nM for 45 minutes or pre-treated with OA 10 nM for 30 minutes prior to the addition of OA 10 nM and 1 μM β-AR agonist for 15 minutes. Cells were fixed and stained for phospho-ERK (white) as described. Three independent experiments from two separate cell strains were performed.

In untreated keratinocytes phospho-ERK is localized to the leading edge of the lamellipodia, a novel finding in keratinocytes. Robust nuclear and peri-nuclear phospho-ERK staining is also observed. β2-AR activation completely prevents the localization of ERK to the leading edge and decreases the nuclear/peri-nuclear staining.

The effects of β2-AR activation on keratinocyte migration, proliferation and cytoskeletal conformation are optionally PP2A-dependent (see, e.g., FIGS. 1-2); thus, whether β2-AR activation induced alteration in phospho-ERK localization had a similar PP2A dependence was investigated. Treatment of cells with OA, the PP2A inhibitor, has no effect on the level of ERK phosphorylation or localization within keratinocytes. However, OA pre-treatment prevents both the loss of lamellipodial localization of phospho-ERK and its decrease in nuclear/peri-nuclear areas Observed in the presence of β2-AR agonist, confirming that the β2-AR-mediated alteration in phospho-ERK localization is also PP2A-dependent.

β2-AR Activation Delays the Re-Epithelialization of Human Skin Wounds

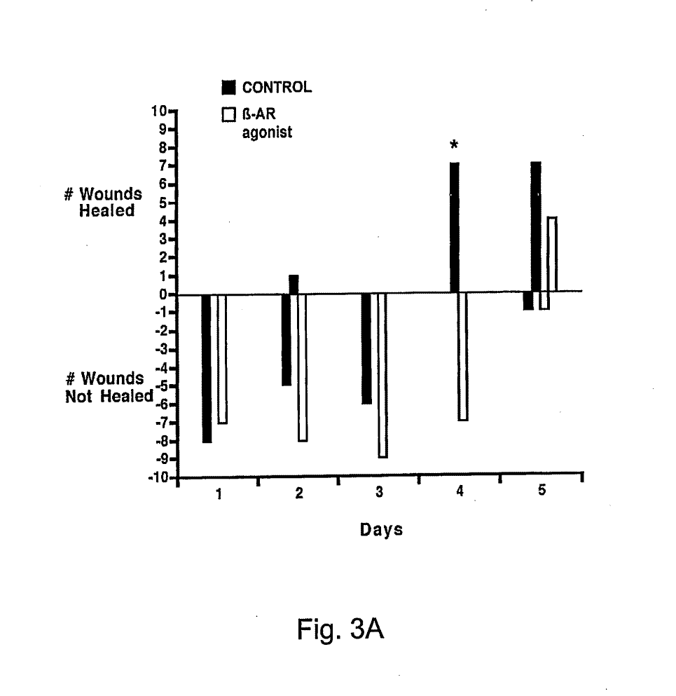

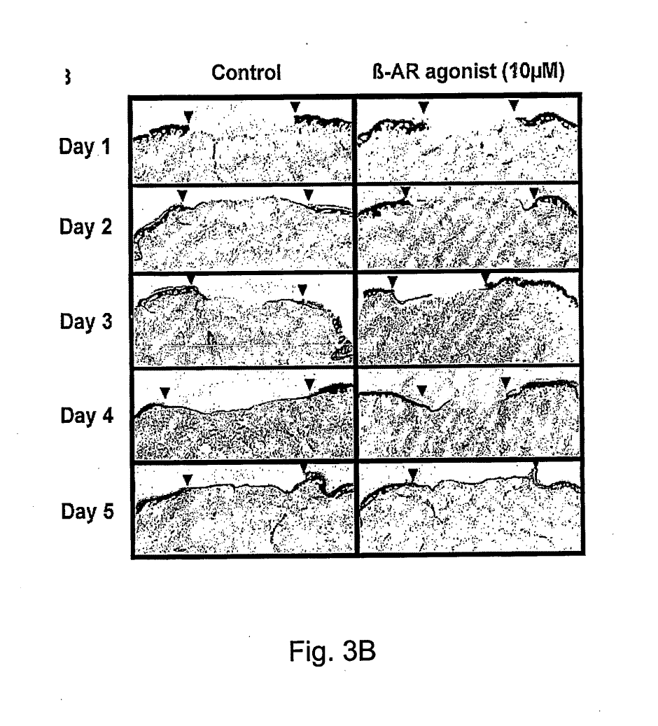

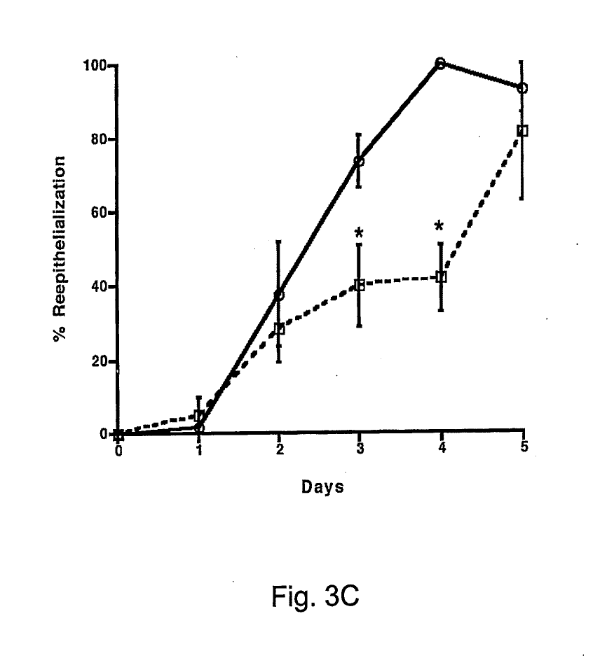

Since β2-AR activation is both anti-motogenic and anti-mitogenic in human keratinocytes, whether wound re-epithelialization, which is essential for wound healing (Martin, supra), could be impaired by these agents was investigated. Human skin was wounded and the wounds allowed to re-epithelialize in explant culture. Addition of β-AR agonist to the healing wound significantly delays healing by 24 hours. All control, untreated, wounds are completely healed by day 4, whereas β2-AR agonist-treated wounds heal by day 5 at the earliest (FIG. 3 Panel A, p<0.005). Hematoxylin and eosin-stained sections from control and β2-AR agonist-treated wounds, days 1-5 are shown in FIG. 3 Panel B. The percentage of re-epithelialization was calculated for each wound. β-AR agonist treatment significantly decreases the wound re-epithelialization by 34% and 58% after 3 and 4 days in culture, respectively (FIG. 3 Panel C). These results demonstrate that β2-AR activation delays wound re-epithelialization in normal human skin.

Wounds, 3 mm in diameter, were generated in excised human skin, cultured in the presence or absence of 10 μM isoproterenol, fixed and stained every day after wounding as described. Re-epithelialization was determined using light microscopy. A (+) score was given to a healed wound and a (−) score to any unhealed wounds. Specimens that were damaged in the histologic process or otherwise non interpretable were excluded from the study. Scores from experiments performed in triplicate on excised skin from 3 different individuals are graphically represented in FIG. 3 Panel A (* p<0.005, using the 2-tailed Fisher's exact test). Images of untreated (control) and β-AR agonist-treated wounds fixed on days 1-5 are presented in FIG. 3 Panel B at 10× magnification. Arrows indicate the wound margin. The percent re-epithelialization was calculated for each wound, and data was analyzed using the Student's t test and represented graphically in FIG. 3 Panel C; control wounds are represented by circles, 1 μM β-AR agonist treated wounds by squares, *P<0.05. Data is combined from three independent experiments, performed in triplicate on excised skin from three different individuals.

β2-AR Activation Decreases the Epidermal Wound-Induced Phosphorylation of ERK

ERK activation is known to play a role in wound healing. Mechanical injury of confluent keratinocyte cultures activates ERK (Turchi et al., supra), and conversely, inhibition of ERK causes a delay in rabbit corneal epithelial wound healing (Sharma et al., supra). To investigate whether the mechanism for the β2-AR-mediated delay in re-epithelialization could involve decreased ERK activation in the wounded epidermis, ERK phosphorylation levels were examined.

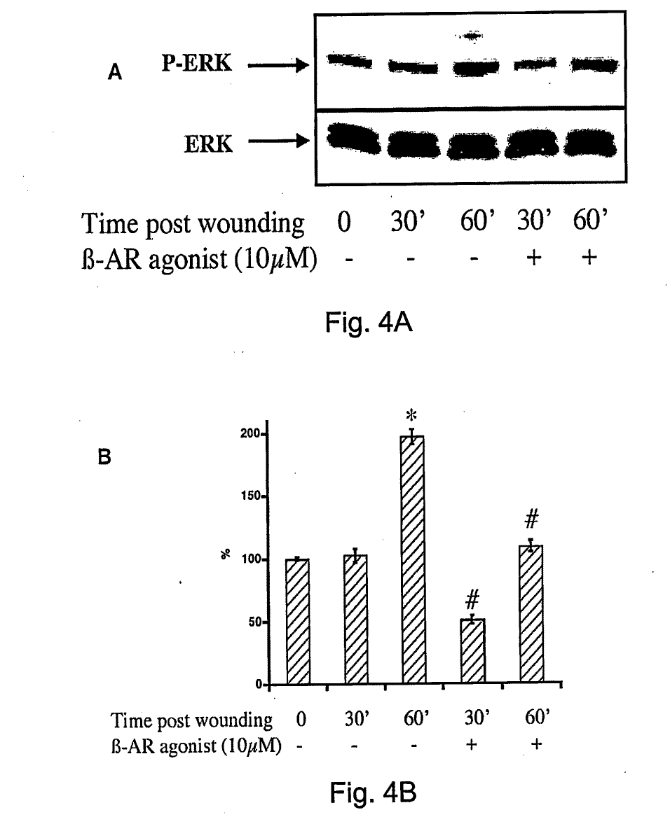

To determine the phosphorylation state of ERK in wounded skin, the excised 3 mm skin wound discs were either frozen immediately (time 0) or pre-incubated in FM for 30 or 60 minutes in the presence or absence of 10 μM β-AR agonist and processed as described. Immunoblots were probed with either an anti-ERK or anti-phospho-ERK antibody (FIG. 4 Panel A). Three blots from separate experiments were scanned for p-ERK (FIG. 4 Panel B) and densitometry performed using NIH Image 1.62. Data was averaged, statistically analyzed and represented graphically. Values plotted are means±SEM (n=3). * P<0.01 between 60′ after wounding and control (0). #P<0.01 between control 30′/60′ and β-AR agonist 30′/60′, respectively. The data shown is combined from three independent experiments performed on excised skin from three different individuals.

In order to study the activation state of ERK in wounded human skin, levels of phospho-ERK were assessed in periwound epidermis. Within 60 minutes of wounding, the phosphorylation of ERK increases two-fold in the periwound epidermis, while the total level of ERK remains unchanged (FIG. 4 Panel A). β2-AR activation decreases the wound-induced phosphorylation of ERK, so that at 30 minutes after β-agonist addition levels of phospho-ERK are significantly lower than the level of ERK phosphorylation detected immediately after wounding. β-agonist treatment decreases the wound-induced increase in phosphorylation of ERK by 80% 60 minutes post wounding (FIG. 4 Panel B), providing convincing evidence that the β2-AR activation-induced delay in human skin re-epithelialization is associated with a decrease in wound-induced epidermal ERK phosphorylation, optionally necessary for efficient wound closure.

β2-AR and Wound Repair

Activation of β2-AR in keratinocytes increases the activity of the phosphatase, PP2A, resulting in a decrease in phosphorylated ERK along with a reduced rate of cell migration (Pullar et al., supra). Data in this example illustrates that activation of β2-AR significantly diminishes capacity for β-AR agonist-treated human skin to re-epithelialize a wound. β2-AR activation remodels the keratinocyte actin cytoskeleton, from that of an actively migratory cell to that of a statically adherent one, with a dense network of actin fibers just beneath the plasma membrane and abundant large vinculin-rich focal adhesions. Both the β-AR agonist-induced cytoskeletal changes and the impairment in migration are reversed when the cells are pre-treated with the phosphatase inhibitor, OA, demonstrating that these events are mediated by the phosphatase PP2A.

β-AR agonists not only inhibit keratinoctye migration, but also keratinoctye proliferation. Both keratinoctye migration and proliferation are typically required for cutaneous wound repair, and indeed, a significant delay in the re-epithelialization of human skin wounds treated with the β-AR agonist isoproterenol is observed. The experiments in this example illustrate specific changes in keratinocyte biology induced by β-AR agonists and the resultant impairment in the process of wound repair, implicating the β2-AR signaling pathway as a regulator of human cutaneous wound repair.

Of the three subtypes of β-AR, optionally only the β2-AR subtype is expressed on keratinocytes, dermal fibroblasts, and melanocytes. However, their function within the skin has been elusive. As noted above, defects in keratinocyte β2-AR density or post-receptor signaling have been observed in both atopic dermatitis and psoriasis, suggesting that the receptor plays a role in epidermal homeostasis. Additionally, calcium concentrations within the epidermis regulate both keratinocyte differentiation state and β2-AR density within the epidermis, (Schallreuter et al (1993) and (1995), both supra, and Gazith and Reichert (1982) High affinity membrane receptors in cultured human keratinocytes. I. The beta-adrenergic receptors. Br J Dermatol 107 Suppl 23:125-133), suggesting that β2-ARs play a role in the differentiation process in human skin. Keratinocytes can synthesize the β-AR catecholamine ligands epinephrine and norepinephrine, thus creating a self-contained epidermal signaling network of comprised of ligand-producing and receptor-expressing cells. Evidence for this network's regulatory role in skin was provided by Gilbro et al, who demonstrated that β2-AR-expressing epidermal melanocytes can respond to keratinocyte-generated catecholamines with increased melanogenesis (Gillbro et al., supra).

The data presented in this example implicate the cutaneous β2-AR network in the process of wound healing. β2-AR regulation of human wound healing has not previously been examined, although earlier work by Donaldson et al (supra) suggested that β-AR agonists may impede this process in newt limbs. However, subsequent work investigating the effects of β2-AR activation on wound repair in other epithelia has yielded conflicting results, with β-AR blockade reported to either enhance (Reidy et al. supra) or delay (Haruta et al. and Liu et al., both supra) corneal epithelial wound healing. Similarly, there are reports of both increased (Salathe (2002) Effects of beta-agonists on airway epithelial cells. J Allergy Clin Immunol 110:S275-281; Spurzem et al. (2002) Activation of protein kinase A accelerates bovine bronchial epithelial cell migration. Am J Physiol Lung Cell Mol Physiol 282:L1108-1116; Murphy et al. (1998) Effect of norepinephrine on proliferation, migration, and adhesion of SV-40 transformed human corneal epithelial cells. Cornea 17:529-536; and Masur et al. (2001) Norepinephrine-induced migration of SW 480 colon carcinoma cells is inhibited by beta-blockers. Cancer Res 61:2866-2869) and decreased (Chen et al. and Pullar et al., both supra) cell motility in response to β2-AR activation. Therefore, it is clear that the response to β2-AR activation, in terms of cell motility and thus its contribution to wound repair, can be highly tissue specific. The current work directly demonstrates the effect of β2-AR activation in human skin. Proliferation of keratinocytes behind the advancing epithelial tongue is also typically required for re-epithelialization (Singer and Clark supra) and prior reports of β2-AR activation enhancing epithelial cell proliferation (Nishimura et al. (1998) [Effect of salbutamol on proliferation of human bronchial epithelial cells: role of MAP kinase]. Nihon Kokyuki Gakkai Zasshi 36:428-432), therefore, suggested another mechanism for β2-AR modulation of wound healing. However, the results described in this example demonstrate that in human keratinocytes β2-AR activation does not enhance, but rather, inhibits cell proliferation, which can contribute to the observed impairment of skin wound epithelialization.

Efficient cell migration, typically required for wound repair, is dependent on temporally and spatially controlled reorganization of the actin cytoskeleton (Pantaloni et al. supra). Within hours of injury, skin wound keratinocytes undergo phenotypic alterations including the formation of a fine and diffuse actin network at the advancing lammellipodium to allow for cell migration (Gabbiani et al. (1978) Cytoplasmic filaments and gap junctions in epithelial cells and myofibroblasts during wound healing. J Cell Biol 76:561-568 and Kubler and Watt (1993) Changes in the distribution of actin-associated proteins during epidermal wound healing. J Invest Dermatol 100:785-789). The expression of integrin receptors on the cell surface stabilizes the lamellipodium (Frank and Carter (2004) Laminin 5 deposition regulates keratinocyte polarization and persistent migration. J Cell Sci 117:1351-1363) and allows the migrating keratinocytes to interact with the variety of extracellular matrices (ECMs) found in the wound site, including fibronectin, vitronectin, stromal type I collagen and fibrin (Larjava et al. (1993) Expression of integrins and basement membrane components by wound keratinocytes. J Clin Invest 92:1425-1435). Integrin-mediated adhesion to ECMs at focal adhesion sites leads to the sequential activation of focal adhesion kinase and ERK, subsequently promoting migration (Klemke et al., supra). Since β2-AR activation is anti-motogenic, the actin cytoskeleton and focal adhesion sites were examined. β2-AR activation induced dramatic changes in both. While untreated cells possessed a polarized migratory phenotype, with a fine actin-rich lamellipodia, containing discrete focal contacts, characteristic of the migratory phenotype (Small et al. (1999) Cytoskeleton cross-talk during cell motility. FEBS Lett 452:96-99), β-AR agonist treated cells showed prominent actin stress fibers restricted to the cell periphery, together with increased numbers of large vinculin-rich focal adhesions characteristic of non-motile cells (Beningo et al. supra). The migratory phenotype was restored by the PP2A inhibitor, OA, indicating that the β2-AR-mediated alterations in the keratinocyte cytoskeletal conformation, as well as the changes in migration speed, were PP2A-dependent. It appears, therefore, that a β2-AR-mediated alteration in cytoskeletal conformation retards keratinocyte migration, which can contribute to impaired wound re-epithelialization.

A growing body of evidence from rat embryo fibroblasts, colon epithelial cells, and pancreatic carcinoma cell lines suggests that ERK may be translocated to focal contact sites at the lamellipodial edge during cell migration (Fincham et al. (2000) Active ERK/MAP kinase is targeted to newly forming cell-matrix adhesions by integrin engagement and v-Src. Embo J 19:2911-2923; Brunton et al. (2001) The protrusive phase and full development of integrin-dependent adhesions in colon epithelial cells require FAK- and ERK-mediated actin spike formation: deregulation in cancer cells. Neoplasia 3:215-226; and Stahle et al. (2003) Mechanisms in LPA-induced tumor cell migration: critical role of phosphorylated ERK. J Cell Sci 116:3835-3846). The novel finding of localization of phospho-ERK to the leading edge of the lamellipodium in migrating keratinocytes was described above. This localization is disrupted by β2-AR activation and restored by OA, indicative of a PP2A-dependent mechanism. Although the exact function of phospho-ERK at the lamellipodial edge remains to be elucidated, direct interactions between ERK and 135, 136 integrins (Ahmed et al. (2002) Direct integrin alphavbeta6-ERK binding: implications for tumor growth. Oncogene 21:1370-1380) and paxillin (Liu et al. (2002) Hepatocyte growth factor induces ERK-dependent paxillin phosphorylation and regulates paxillin-focal adhesion kinase association. J Biol Chem 277:10452-10458) suggest an important role for ERK in integrating cell adhesion and receptor-mediated signaling in the control of cell migration. Yet again, β2-AR activation appears to disrupt an event that plays a part in normal cell migration.

Evidence for the role of β2-AR in human wound repair is the direct demonstration that activation of β2-AR receptors in excised, wounded skin significantly delays its re-epithelialization (FIG. 3). Using tissue confers the advantages of a normal extracellular matrix and the three dimensional geometry of the healing wound, not found in scratch assays or other assays using cultured cells. Additionally, β2-AR activation was demonstrated to decrease ERK phosphorylation within the wounded epidermis. Since ERK is activated upon mechanical injury of confluent keratinocyte (Turchi et al. supra) and MDCK cultures (Matsubayashi et al. (2004) ERK activation propagates in epithelial cell sheets and regulates their migration during wound healing. Curr Biol 14:731-735) and inhibition of ERK delays rabbit corneal epithelial wound healing (Sharma et al. supra), the β2-AR-mediated decrease in ERK phosphorylation may play a role in the β2-AR-mediated delay in re-epithelialization.

Although β-AR agonists and antagonists are widely used drugs in the treatment of asthma and cardiologic disease, respectively, there have been no specific observations regarding the ability of patients using these agents to heal wounds. Indirect observations that related to endogenous and exogenous β-AR agonists and antagonists and wound healing include the observations that psychological stress, a condition that elevates systemic catecholamine levels (Nankova and Sabban (1999) Multiple signaling pathways exist in the stress-triggered regulation of gene expression for catecholamine biosynthetic enzymes and several neuropeptides in the rat adrenal medulla. Acta Physiol Scand 167:1-9), is associated with delayed skin wound healing (Detillion et al. (2004) Social facilitation of wound healing. Psychoneuroendocrinology 29:1004-1011). Denda et al. have demonstrated that emotional stress results in an impaired skin permeability barrier (Denda et al. (2000) Stress alters cutaneous permeability barrier homeostasis. Am J Physiol Regul Integr Comp Physiol 278:R367-372), and conversely, that topical application of β-AR antagonists can accelerate skin barrier recovery after barrier disruption (Denda et al. (2003) Beta2-adrenergic receptor antagonist accelerates skin barrier recovery and reduces epidermal hyperplasia induced by barrier disruption. J Invest Dermatol 121:142-148). β-AR antagonists are widely used in the post-burn wound recovery period (e.g., for cardiovascular complications), and a retrospective outcome analysis by Arbabi et al suggested a shorter time for burn wound healing in a cohort of patients that received β-AR antagonists during their hospital stay (Arbabi et al. (2004) Beta-blocker use is associated with improved outcomes in adult burn patients. J Trauma 56:265-269; discussion 269-271). However, differences between the treated and untreated patient cohorts were not statistically significant.

Defining pathways that regulate the wound healing process provides the potential for developing new therapeutic approaches. The current finding, that β2-AR activation significantly delays wound re-epithelialization and decreases the wound-induced increase in epidermal phospho-ERK, indicates that treatment with β2-AR agonists and antagonists is a viable therapeutic approach to modulating epithelialization and/or wound repair, e.g., in skin.

### Example 2

### β2-Adrenergic Receptor Antagonist Speeds Wound Healing

The following sets forth a series of experiments that demonstrate use of β2-AR antagonists to increase the rate of re-epithelialization in cell culture and in human skin explants.

β2-AR antagonists promote wound re-epithelialization in a “chronic” human skin wound-healing model. β-AR antagonists increase ERK phosphorylation, the rate of keratinocyte migration, electric field-directed migration and ultimately accelerate human skin wound re-epithelialization. The experiments described in this example demonstrate that keratinocytes express two key enzymes required for catecholamine (β-AR agonist) synthesis, tyrosine hydroxylase and phenylethanolamine-N-methyl transferase, both localized within keratinocyte cytoplasmic vesicles. The experiments also confirm the synthesis of epinephrine by measuring the endogenously synthesized catecholamine in keratinocyte extracts. The previous example demonstrated that β-AR agonists delay wound re-epithelialization; this example demonstrates that β-AR antagonists accelerate wound re-epithelialization. Without intending to be limited to any particular mechanism, the β-AR antagonist-mediated augmentation of wound repair can be due to β2-AR blockade, preventing the binding of endogenously synthesized epinephrine.

β-adrenergic receptors (β-ARs) are expressed on a wide variety of tissues, and are recognized as pivotal functional regulators of the cardiac, pulmonary, vascular, endocrine and central nervous systems. Although their expression in human skin was noted over 30 years ago (Tseraidis and Bavykina (1972) Vestn Dermatol Venerol 46:40-45), only recently has their functional significance in this tissue been recognized. The β2-AR subtype is the only subtype of β-ARs currently known to be expressed on the membranes of the major cell types in skin: keratinocytes, fibroblasts and melanocytes (Schallreuter et al. (1993) Arch Dermatol Res 285:216-220; Steinkraus et al. (1992) J Invest Dermatol 98:475-480; Steinkraus et al. (1996) Arch Dermatol Res 288:549-553; McSwigan et al. (1981) Proc Natl Acad Sci USA 78:7670-7673; and Gillbro et al. (2004) J Invest Dermatol 123:346-353). Keratinocytes express a high level of β2-ARs, which appear to play a role in cutaneous homeostasis. Interestingly, aberrations in either keratinocyte β2-AR function or density have been associated with cutaneous disease. Keratinocytes derived from patients with atopic eczema display a point mutation in the β2-AR gene and a low β2-AR density (Schallreuter (1997) J Investig Dermatol Symp Proc 2:37-40). In psoriasis, keratinocytes within the psoriatic lesions demonstrate a low cAMP response to β2-AR activation (Eedy et al. (1990) Br J Dermatol 122:477-483). These findings point to a role for the β2-AR in maintaining epidermal function and integrity. The data provided in this example supports a role for the β2-AR in regulating wound repair as well.

As noted above, cutaneous wound healing is a complex and well-orchestrated biological process requiring the coordinated migration and proliferation of both keratinocytes and fibroblasts, as well as other cell types. Wounding the epidermis generates cytokines, growth factors and proteases and initiates the synthesis of extra cellular matrix components, all of which can regulate the processes of keratinocyte migration and proliferation, involved in re-epithelialization (Martin (1997) Science 276:75-81 and Singer and Clark (1999) N Engl J Med 341:738-746). Upon injury, cells migrate directionally towards the center of the wound bed to initiate repair and restore epithelial integrity. Many cues play a role in wound-induced keratinocyte directional migration including contact inhibition and chemotaxis (Wilson et al. (2001) Prog Retin Eye Res 20:625-637 and Lu et al. (2001) Exp Biol Med (Maywood) 226:653-664). Additionally, electric fields (EFs) play an important role in epithelial cell directional migration and wound healing (Zhao et al. (1996) Invest Opthalmol Vis Sci 37:2548-2558; Zhao et al. (1996) J Cell Sci 109 (Pt 6):1405-1414; Zhao et al. (1997) Curr Eye Res 16:973-984; Chiang et al. (1992) Exp Eye Res 54:999-1003; Sta Iglesia and Vanable (1998) Wound Repair Regen 6:531-542; and Song et al. (2002) Proc Natl Acad Sci USA 99:13577-13582). Indeed, increasing or decreasing corneal wound currents pharmacologically correlates directly with an increased or decreased rate of healing in the rat cornea (Reid et al. (2005) Faseb J 19:379-386).

One early study demonstrated that β-AR agonists delay skin wound healing in newt limbs (Donaldson and Mahan (1984) Comp Biochem Physiol C 78:267-270). Subsequent studies in other epithelia, however, have yielded conflicting results. For example, β-AR antagonists have been reported to either delay (Haruta et al. (1997) Eur J Opthalmol 7:334-339 and Liu et al. (1990) J Ocul Pharmacol 6:101-112) or enhance (Reidy et al. (1994) Br J Opthalmol 78:377-380) corneal epithelial wound healing. Recently, by measuring transepidermal water loss, Denda et al. reported that the β-AR could modulate epidermal barrier permeability (Denda et al. (2003) J Invest Dermatol 121:142-148).

The experiments described in this example investigate the effects of β-AR antagonists on scratch wound healing, keratinocyte single cell migration, ERK phosphorylation, keratinocyte galvanotaxis, cytoskeletal organization, proliferation and ultimately, human skin wound re-epithelialization in a “chronic” wound healing model. The results demonstrate that the β-AR antagonist is pro-motogenic, promoting human skin re-epithelialization. Expression of protein for two key catecholamine (β-AR agonist) synthesis enzymes is detected; the enzymes are localized within keratinocyte cytoplasmic granules/vesicles. The results also indicate that keratinocytes synthesize epinephrine. The results thus indicate the presence of an endogenous β-AR mediator network in the skin that upon blockade accelerates wound healing.

### Experimental Procedures

Materials

Timolol (β-adrenergic antagonist) and the anti-vinculin antibody were purchased from Sigma (St. Louis, Mo.). ICI 118,551 (β-adrenergic antagonist) was purchased from Tocris (Ellisville, Mo.). The anti-ERK (#9102), anti-phospho-ERK (#9101) and anti-rabbit-horseradish peroxidase linked secondary antibodies were purchased from Cell Signaling Technology (Beverly, Mass.). The anti-tyrosine hydroxylase (TH) antibody (AB152) was purchased from Chemicon (Temecula, Calif.). The anti-phenylethanolamine-N-methyl transferase (PNMT) antibody was purchased from Biogenesis (Brentwood, N.H.).

Keratinocyte Growth

Human keratinocytes were isolated from neonatal foreskins as reported previously (Isseroff et al. (1987) J Lipid Res 28:1342-1349), under an approved exemption granted by the Internal Review Board at University of California, Davis, and cultured using a modification of the method of Rheinwald and Green ((1975) Cell 6:331-343). Cells were grown in keratinocyte growth medium (KGM) (Epilife, 0.06 mM Ca²⁺), supplemented with human keratinocyte growth supplement (0.2 ng/ml EGF, 5 μg/ml insulin, 5 μg/ml transferrin, 0.18 μg/ml hydrocortisone and 0.2% bovine pituitary extract) (Cascade Biologics, Inc., Portland, Oreg.) and antibiotics (100 U/ml penicillin, 100 μg/ml streptomycin, and 0.25 μg/ml amphotericin) (Gemini Bio-Products, Inc., Calabasas, Calif.) at 37° C. in a humidified atmosphere of 5% CO₂. Cell strains isolated from at least three different foreskins were used in all experiments, performed with sub-cultured cells between passages 3-7.

Scratch Assay

Cells were grown to confluence in KGM on 35 mm culture dishes (Fisher Scientific, Pittsburgh, Pa.). Cells were either untreated (control) or treated with β-AR antagonist (ICI 118,551) at a concentration specific for the β2-AR (Bilski et al. (1983) J Cardiovasc Pharmacol 5:430-437), 10 nM, in KGM at time 0. A sterile pipette tip was used to scratch a 1 mm-wide wound along the center of the dish or cover slip and a demarcated area of the wound was photographed on an inverted Nikon Diaphot microscope at the time of wounding (time 0) and at 16 hours post wounding (Haas et al. (1990) J Invest Dermatol 94:822-826) at 10× magnification.

Single Cell Migration Assay

Glass bottomed 35 mm dishes (MatTek Corporation, Ashland, Mass.), were coated with collagen I (60 μg/ml) (Cohesion Technologies, Palo Alto, Calif.) in PBS for 1 hour at 37° C. Keratinocytes were plated at a density of 50 cells/mm² for 2 hours at 37° C. Cells were incubated with KGM alone (control) or with KGM containing 20 μM β-AR antagonist (timolol) at time 0. The 35 mm glass-bottomed dishes were placed in a heating chamber, designed to maintain the medium between 35-37° C., and secured to the stage of an inverted Nikon Diaphot microscope. Individual cell migration was monitored over a 1 hour period at 37° C., as described previously (Pullar and Isseroff (2005) J Cell Sci 118:2023-2034). Time-lapse images of the cell migratory response were digitally captured every 10 minutes by Q-Imaging Retiga-EX cameras (Burnaby, BC, Canada) controlled by a custom automation written in Improvision Open Lab software (Lexington, Mass.) on a Macintosh G4. After each cell's center of mass was tracked using the Open Lab software, migration speed and distance were calculated and imported to Excel (Microsoft Corporation, Redmond, Wash.). “Distance” is the average total distance in μm that the cells travel in a one-hour period of time. “Speed” is the average speed in μm/min that the cells travel in a one-hour period of time. Significance was taken as P<0.01, using Student's T test (unpaired) to compare the means of two cell populations.

Cell Treatments for Immunoblotting

1×10⁶ plated keratinocytes were incubated with either KGM alone (control and lysates for catecholamine synthesis enzyme detection) or KGM containing 20 μM β-AR antagonist (timolol) for 5-60 minutes. Cells were placed immediately on ice, washed twice with ice cold phosphate buffered saline (PBS) containing phosphatase inhibitors (50 mM NaF and 1 mM Na₃VO₄) and scraped in 50 μl lysis buffer; (PBS containing 0.5% Triton X-100, 50 mM NaF, 1 mM Na₃VO₄, leupeptin 10 μg/ml, aprotinin 30 μg/ml, PMSF 200 pepstatin A 10 μg/ml). The lysates were transferred into 1.5 ml tubes, incubated on ice for 20 minutes and then centrifuged at 14,000 g for 10 minutes at 4° C. (Pullar et al. (1996) Immunol 157:1226-1232). The protein concentration of the samples was determined using the Bradford Assay (Bio-Rad Laboratories, Hercules, Calif.). The supernatants were electrophoresed immediately on 10% polyacrylamide Tris-HCl gels (Bio-Rad Laboratories, Hercules, Calif.) or stored at −80° C.

Five μg (P-ERK blots) or thirty-five μg (catecholamine synthesis enzyme blots) of each protein sample was added to an equal volume of 2× reducing sample loading buffer (0.0625M Tris-HCl pH 6.8, 3% SDS, 10% glycerol, 5% β-ME) and electrophoresed on 10% polyacrylamide Tris-HCl gels. Proteins were transferred to Immobilon membranes and immunoblotted with an anti-ERK (#9102), phospho-ERK (#9101), TH or PNMT antibody at a concentration recommended by the manufacturer. The immunoblots were developed by enhanced chemiluminescence (ECL) according to the manufacturer's instructions (Amersham Pharmacia Biotech, Piscataway, N.J.). Densitometry was performed on scanned images using NIH Image 1.62.

Galvanotaxis

The galvanotaxis chamber construction and DC application were performed as described previously (Pullar et al. (2001) Cell motility and the cytoskeleton 50:207-217). Briefly, the galvanotaxis chamber is composed of a rectangular plexiglass frame with two medium reservoirs on opposite sides to which a 45×50 mm piece of No. 1.5 glass coverslip is attached to form the chamber bottom. This allows for continual observation of the plated cells on an inverted microscope. The keratinocytes are plated onto the collagen-coated center of the chamber between two coverslip spacers 25×10 mm. A third 25 mm² cover slip is placed on top, straddling the two spacer cover slips and covering the cells plated on the collagen-coated center panel. This third coverslip rests approximately 100 to 105 μm above the center panel and is sealed on top of the spacer cover slips with silicone high vacuum grease, Dow Corning (Midland, Mich.). This small height is chosen to minimize the cross-sectional area through which current flows. A small cross-section creates a high resistance pathway resulting in a higher voltage gradient for a fixed current. The aqueduct allows for medium and current flow across the cells. The voltage across the coverslip is measured using a voltmeter via silver-sliver chloride (Ag—AgCl) wire electrodes inserted into both medium reservoirs on either side of the center panel. Six cm long 2% agar/phosphate buffered saline-filled pieces of polypropelene tubing connect each end of the chamber to a medium-filled well in which the Ag—AgCl electrodes are placed, to separate possible electrode byproducts from the cells themselves. The current is measured with an ammeter in series and only chambers for which the current flow is kept below 0.6 mA are used, to minimize joule heating. Furthermore, temperature of the medium in the chambers is maintained at 36° C. by placing the chamber on a metal plate heated to and maintained at 39° C. Temperature is continuously monitored during the experiment using a YSI 400 analog temperature probe (Yellow Springs Instrument Co., Inc., Yellow Springs, Ohio) directly attached to the metal plate, and does not vary by more than 1° C. over the course of the experiment.

Time Course Observation and Data Analysis

The galvanotaxis chambers rest on inverted Nikon microscopes. Time-lapse images of the cell migratory response are digitally captured every 10 minutes over a one-hour period by a Q-Imaging Retiga-EX cameras (Burnaby, BC, Canada) controlled by a custom automation written in Improvision Open Lab software (Lexington, Mass.) on a Macintosh G4. After each cell's center of mass is tracked using the Open Lab software, directionality of migration is calculated and imported to Excel (Microsoft Corporation, Redmond, Wash.). Cosine θ describes the direction of migration and is a measure of the persistence of cathodal directedness, where θ is the angle between the field axis and the vector drawn by the cell migration path. The average cosine θ=Σᵢ cos θᵢ/N, where Σᵢ is the summation of 70-72 individual cells from at least three different cell strains. Angle zero) (θ=0°) is assigned to the negative pole (cathode) and increasing angles assigned in a clockwise manner, with θ=180° aligned with the positive pole (anode). Therefore, the cosine θ will provide a number between −1 and +1 and the average of all of the separate cell events yields an index of directional migration. For example, if a cell were to move directly to the negative pole, the angle (θ)=0° and the cosine θ=1. “Cosine”, therefore, refers to the average directional migration index of separate cell migration events at the end of a one-hour period. Results are given as average cosine θ±the standard error of the mean (s.e.m.). Significance is taken as p<0.01, using Student's T test (unpaired) to compare the means of two cell populations.

Immunofluorescent Staining and Microscopy

Sterile glass cover slips were transferred into 12 well dishes and collagen-coated with 60 μg/ml collagen I in KGM for 1 hour at 37° C. as described. Cover slips were washed three times with KGM and 3×10⁴ cells were added per well and allowed to attach overnight. Cells were untreated or treated with 20 μM β-AR antagonist for 15 minutes. Cover slips were processed at room temperature unless otherwise noted. Cover slips were washed twice in PBS and fixed for 10 minutes in 4% paraformaldehyde. Cover slips were washed twice in PBS between each step. Cells were permeabilized for 5 minutes with 0.1% Triton-X-100/PBS, blocked with 5% goat serum/PBS (vinculin) or 5% horse serum/PBS (TH and PNMT) for 20 minutes. Primary monoclonal anti-vinculin antibody was added drop-wise in 1% goat serum/PBS (1:100) and primary anti-TH or anti-PNMT antibody were added drop-wise in 1% horse serum/PBS (1:20) and incubated for 1 hour at 37° C. A goat anti-mouse cy3 antibody (Jackson labs, West Grove, Pa.) (1:100) was added in 1% goat serum/PBS for 1 hour at 37° C. (vinculin) or a donkey anti-rabbit cy3 antibody (Jackson labs, West Grove, Pa.) (1:100) was added in 1% horse serum/PBS for 1 hour at 37° C. (TH and PNMT). Alexa 488-phalloidin (Molecular Probes, Eugene, Oreg.) (1:40 in PBS) was added to the vinculin-stained cover slips for 20 minutes. Standard controls were performed. Cover slips were incubated with the primary antibody alone or the secondary antibody alone to ensure specificity. Finally, Prolong gold anti-fade reagent (Molecular Probes, Eugene, Oreg.) was used according to manufacturer's instructions to mount the cover slips onto glass microscope slides. Slides were viewed on an inverted fluorescent Nikon Diaphot microscope using a 40× pan fluor objective. Images were captured using Q-imaging Retiga-EX cameras (Burnaby, BC, Canada) and pseudo-colored green for Alexa 488 Phalloidin staining (actin) or red for Cy3 staining (vinculin) or visualized in grey scale for TH and PNMT.

Proliferation Assay

Keratinocytes were released from the tissue culture plate by treatment with 0.25. % trypsin/0.1% EDTA (Gibco, Grand Island, N.Y.), resuspended in KGM and counted using a hemocytometer. 5×10⁴ cells were plated per well in a 12 well plate in triplicate and allowed to settle and attach to the plate for 2 hours. Cells were then cultured in the presence or absence of 20 μM β-AR antagonist. Triplicate wells were harvested and counted on days 2, 4, 6, 8. The medium was changed every day. Significance was taken as p<0.01, using Student's t test (unpaired) to compare the means of the cell populations.

“Chronic” Human Skin Wound Healing Assay

A wound healing model developed by Kratz ((1998) Microsc Res Tech 42:345-350) was previously adapted to observe a delay in human skin re-epithelialization in the presence of β-AR agonist (Pullar et al. (2006) Faseb J 20:76-86). Here a “chronic” wound healing model also developed by Kratz ((1998) Microsc Res Tech 42:345-350) has been adapted. Serum content of the medium has been reduced from 10% to 5% to generate a “chronic” wound-healing model, severely delaying the rate of re-epithelialization in control wounds to enable observation of any increase in the rate of re-epithelialization in the presence 10 μM β-AR antagonist.

Normal human skin was obtained from routine breast reductions or abdominoplasties under an approved exemption granted by the Internal Review Board at University of California, Davis. Under sterile conditions, excess subcutaneous fat was trimmed from 6″×3″ sections of skin prior to stretching and pinning onto sterile cork-board. A 3 mm punch (Sklar Instruments, West Chester, Pa.) was used to make wounds in the epidermis and into the superficial dermis and the 3 mm discs of skin were excised using sterile scissors. 6 mm skin discs, with the 3 mm epidermal wound in the center, were excised using a 6 mm biopsy punch (SMS Inc., Columbia, Md.).

To observe any β-AR antagonist-mediated modulation in re-epithelialization, the skin samples were immediately transferred to a 12 well dish and submerged in 2 ml of FM (Dulbecco's Modified Eagle's medium (DMEM) (Gibco, Grand Island, N.Y.) containing 5% fetal bovine serum (Tissue Culture Biologicals, Tulare, Calif.) and antibiotics in the presence or absence of 10 μM β-AR antagonist. The 12 well dishes were incubated at 37° C. in a humidified atmosphere of 5% CO₂. The medium was changed every day. Three biopsies were fixed in 4% neutral buffered formaldehyde every day for 5 days. The formaldehyde fixed biopsies were dehydrated through an ethanol-xylene series and embedded in paraffin. Cross-sections, 5 μM thick, taken from the center of the wound, were stained using the hematoxylin-eosin technique. Re-epithelialization was determined using light microscopy. A (+) score was given to a healed wound and a (−) score to any unhealed wounds. Slides were viewed on an inverted Nikon Diaphot microscope using a 10× objective. Images were captured using Q-imaging Retiga-EX cameras (Burnaby, BC, Canada). Specimens that were damaged in the histologic process or otherwise non interpretable were excluded from the study. Significance was taken as P<0.05, using the 2-tailed Fisher's exact test to compare the number of wounds healed versus unhealed in the absence or presence of β-AR antagonist. The percentage of re-epithelialization was calculated by measuring the linear distance covered by new epithelium and dividing that by the linear distance between the original wound edges. The new epithelium was clearly differentiated from the epithelial wound margin by the presence of a fully stratified epithelium and fully formed stratum corneum in the latter. In order to eliminate observer bias the % re-epithelialization was calculated from randomly numbered pictures of the wounds by a third party.

To confirm that untreated wounds retain the capacity to heal, biopsies previously cultured for 4 days in 5% serum, were cultured in the presence or absence of an additional 5% serum (i.e. 10% total) for a further 4 days prior to fixation and histological processing. Significance was taken as P<0.01, using Student's T test (unpaired) to compare the means of the % re-epithelialization of the control and β-AR antagonist-treated wounds on each day.

Enzyme Immunoassay for the Quantitative Determination of Epinephrine in Small Sample Volumes

1×10⁷ keratinocytes were extracted in 100 μl 10.1N HCl and sonicated on ice for 10 minutes. Extracts from three strains of keratinocytes were tested in triplicate in an epinephrine enzyme immunoassay (EIA) (Biosource, Camarillo, Calif.) according to the manufacturers instructions. Briefly, the assay kit provides materials for the quantitative measurement of epinephrine. Epinephrine is extracted using a cis-diol specific affinity gel, then acylated to N-acylepinephrine, and after this converted enzymatically during the detection procedure into N-acylmetanephrine. The competitive ETA uses the microtiter plate format. Epinephrine is bound to the solid phase of the microtiter plate. Acylated epinephrine and solid phase bound epinephrine compete for a fixed-number of antiserum binding sites. When the system is in equilibrium, free antigen and free antigen-antiserum complexes are removed by washing. The antibody bound to the solid phase catecholamine is detected by an anti-rabbit IgG peroxidase conjugate using TMB as a substrate. The reaction is monitored at 450 nm with the amount of antibody bound to the solid phase being inversely proportional to the catecholamine concentration in the sample. A set of standards and two controls are included for determination of unknown concentrations (0, 5.6, 19, 83, 306, 1550 pg epinephrine/sample). The linear mean absorbance readings of the standards are plotted on the y-axis versus the log of the concentrations of the standards (pg/sample) on the x-axis and a linear curve fit is applied. The concentration of epinephrine in the unknowns can then be calculated from the slope of the line. To standardize the levels of epinephrine measured in the keratinocyte extracts, a Bradford assay was performed on the extracts, as described, and the level of epinephrine detected was calculated as pg epinephrine per mg protein.

### Results

β2-AR Antagonists Accelerate the Healing of Scratch Wounds in Confluent Keratinocyte Cultures

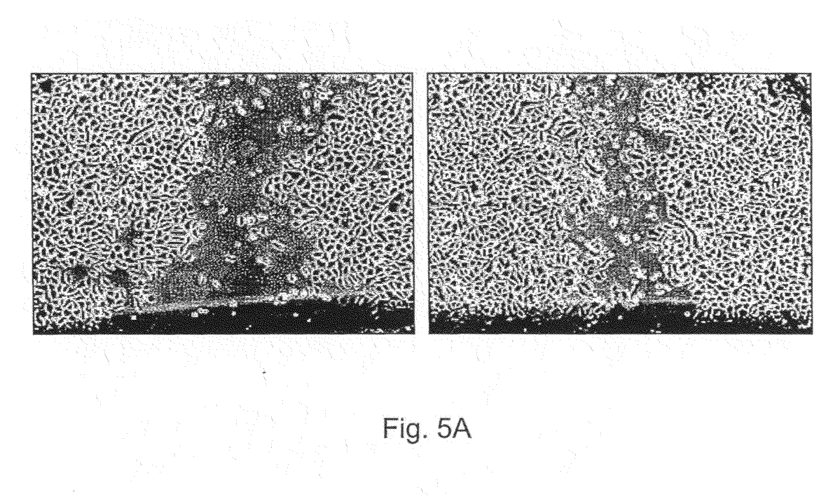

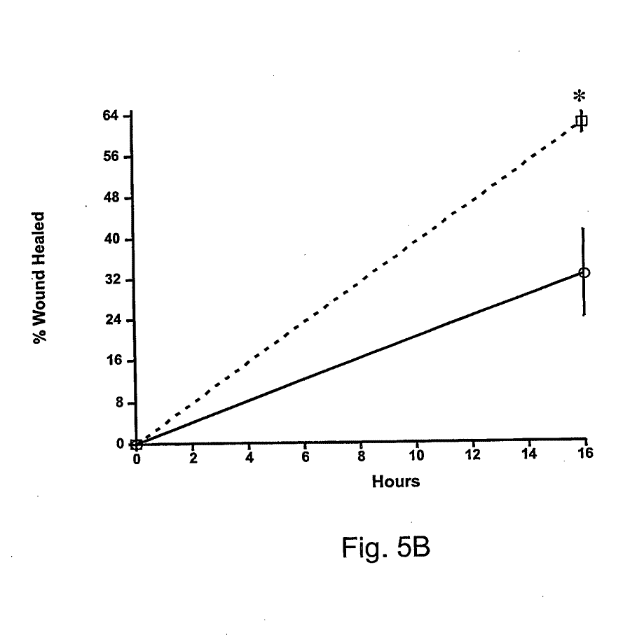

To determine the effect of β-AR antagonists on keratinocyte migration, the ability of keratinocytes to heal a “scratch wound” within a confluent sheet of cells (Pullar et al. (2003) J Biol Chem 278:22555-22562) was measured. Keratinocytes were grown to confluence on collagen-coated plastic dishes as described. Cultures were wounded and KGM alone or KGM containing 10 nMM ICI 118,551 was added at Time 0. A demarcated area of the wound was photographed at the time of wounding (time 0) and again at 16 hours. Photographs of control and β-AR antagonist-treated wounds at time 0 and 16 hours after wounding are represented in FIG. 5 Panel A. The % wound healing was calculated and is represented graphically in FIG. 5 Panel B, for control (◯______) and timolol ( [symbol] --------). The data shown are representative of three independent experiments from three separate cell strains. Values plotted are means+/−SEM (n=3). * P<0.01 between β-AR antagonist and control.

The results demonstrate that a β-AR antagonist, at a concentration specific for β2-AR (Bilski et al. (1983) J Cardiovasc Pharmacol 5:430-437), significantly accelerates scratch wound healing by 1.6 fold. Untreated wounds are only 25% healed within 16 hours whereas β-AR antagonist-treated wounds are 66% healed within the same time frame. A non-specific β-AR antagonist also accelerates keratinocyte scratch wound healing.

β-AR Antagonists Increase the Rate of Keratinocte Single Cell Migration

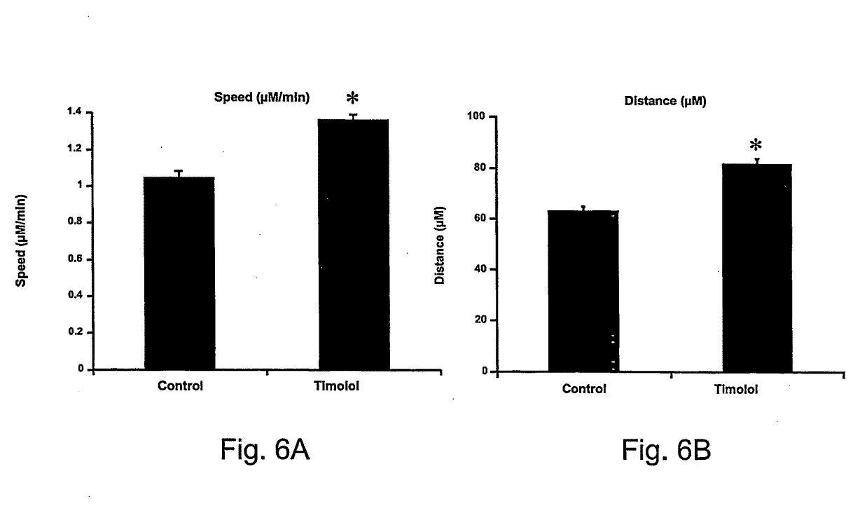

To more precisely measure the effect of β-AR antagonists on motility, the effect of β2-AR blockade on the locomotory speed of individual keratinocytes was observed. Keratinocytes were either untreated or treated with 20 μM β-AR antagonist at time 0 and the migration of each single cell monitored over a one-hour period, as described. The speed of migration and the distance traveled in 1 hour are represented graphically in FIG. 6 Panel A and Panel B, respectively (n=127 (control), n=130 (β-AR antagonist)). The data shown are representative of multiple independent experiments from three separate cell strains. Values plotted are means+/−SEM. * P<0.01 between antagonist and control. The β-AR antagonist significantly increases both keratinocyte migration rate and distance traveled in the one-hour period by 30%.

A β-AR Antagonist Increases ERK Phosphorylation within Minutes in Keratinocytes

ERK plays a pivotal role in pro-migratory signaling pathways and is important for the healing of scratch wounds in confluent monolayers of epithelial cells (Zeigler et al. (1999) J Cell Physiol 180:271-284; Leng et al. (1999) J Biol Chem 274:37855-37861; Glading et al. (2000) J Biol Chem 275:2390-2398; Klemke et al. (1997) J Cell Biol 137:481-492; Wang et al. (2003) Invest Opthalmol Vis Sci 44:244-249; Shanley et al. (2004) Invest Opthalmol. Vis Sci 45:1088-1094; Xu et al. (2004) Invest Opthalmol Vis Sci 45:4277-4283; Turchi et al. (2002) J Invest Dermatol 119:56-63; Matsubayashi et al. (2004) Curr Biol 14:731-735; and Sharma et al. (2003) J Biol Chem 278:21989-21997). Phosphorylation of ERK was therefore examined in antagonist treated cells.

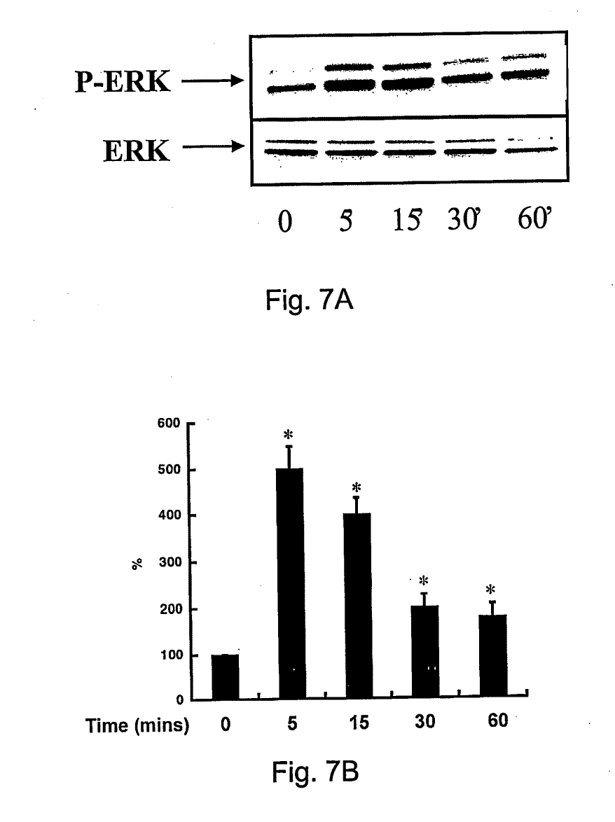

Keratinocytes were cultured in KGM and plated as described. Cells were either untreated (time 0) or incubated with 20 μM β-AR antagonist for up to 60 minutes at 37° C. After treatment, cell lysates were prepared as described, electrophoresed on 10% polyacrylamide gels and transferred to membrane. Identical membranes were immunoblotted with either an anti-phospho ERK or an anti-ERK antibody (FIG. 7 Panel A, top and bottom, respectively, antagonist-treated). The data shown are representative of three independent experiments from three separate cell strains. Three blots from separate experiments were scanned for ERK and p-ERK and densitometry performed using a gel plotting macro in NIH Image 1.62. Data was normalized, averaged, statistically analyzed and represented graphically (FIG. 7 Panel B). Values plotted are means±SEM (n=3). * P<0.01 compared to the control.

The results demonstrate that β-AR antagonist treatment dramatically increases ERK phosphorylation by 5-fold within 5 minutes. ERK phosphorylation remains elevated for up to 60 minutes in the presence of β-AR antagonist while gradually returning towards control levels.

β-AR Antagonists Enhance Keratinocyte EF-Mediated Directionality of Migration

The electric field generated immediately upon wounding (McCaig et al. (2005) Physiol Rev 85:943-978) could be the earliest signal that cells receive to direct their migration into the wound bed. Since the center of the wound is negative with respect to the wound edges (Ojingwa and Isseroff (2003) J Invest Dermatol 121:1-12 and McCaig and Zhao (1997) Bioessays 19:819-826) and keratinocytes migrate towards the negative pole (cathode) in an applied electric field (Nishimura et al. (1996) J Cell Sci 109:199-207), the wound-generated endogenous field likely orients keratinocyte directional migration towards the wound center. Accordingly, the effect of β-AR antagonists on the ability of keratinocytes to sense and respond to an applied electric field of 100 mV/mm was investigated.

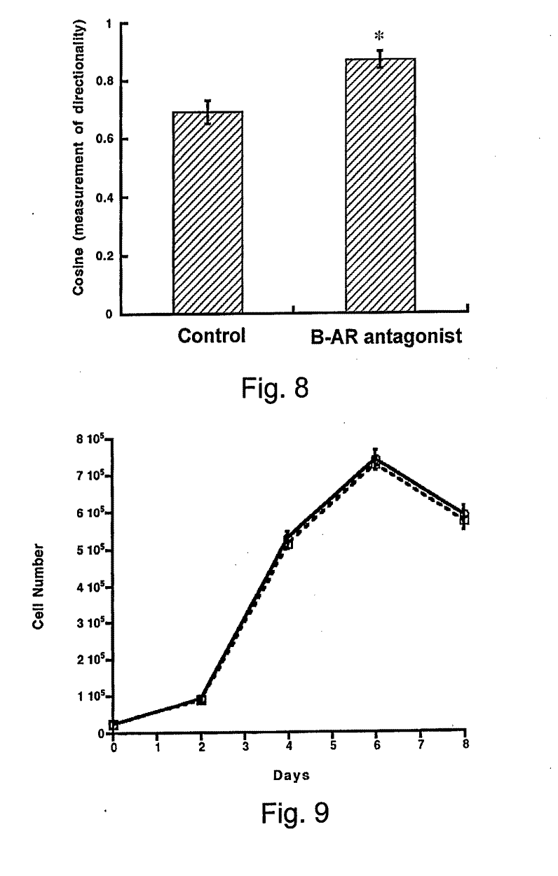

Keratinocytes were seeded at low density in electrotactic chambers, the galvanotaxis chamber was assembled, and a DC EF of 100 mV/mm was applied as described. Galvanotaxis was performed in the absence or presence of 20 μMβ-AR antagonist as described. Time-lapse images were recorded every 10 minutes and analyzed with Improvision software. Cell directedness is represented graphically FIG. 8 (n=70 (control) n=72 (β-AR antagonist)). The data shown are representative of multiple independent experiments with three different strains of keratinocytes. Values plotted are means+/−SEM. * P<0.01 between antagonist and control.

As expected, the β-AR antagonist increases the rate of migration as previously described in FIG. 6. Additionally, β-AR antagonist treatment significantly increases the directionality of migration (cosine) by 26% (FIG. 8).

β-AR Antagonists Preserve the Keratinocyte Pro-Migratory Cytoskeletal Architecture

Actin remodeling plays an important role in cell polarization and motility (Ridley et al. (2003) Science 302:1704-1709 and Pantaloni et al. (2001) Science 292:1502-1506). Actin filaments terminate in focal adhesions (FAs), where several proteins, including vinculin, mediate interactions with the actin cytoskeleton (Burridge and Fath-(1989) Bioessays 10:104-108). FAs mediate the mechanical attachment of cells to the extra cellular matrix and act as signaling centers, capable of regulating gene expression, cell growth and survival (Gilmoreand Burridge (1996) Structure 4:647-651 and Sastry and Burridge (2000) Exp Cell Res 261:25-36). Additionally, small, nascent FAs have been associated with actively migrating cells (Beningo et al. (2001) J Cell Biol 153:881-888). Since β2-AR blockade increases the rate of keratinocyte migration, the actin cytoskeleton was observed for any alterations.

Sterile cover slips were coated with collagen and keratinocytes plated as described. Keratinocytes were left untreated (control) or treated with β-AR antagonist (20 μM) (β-AR antagonist) for 15 minutes. Cells were fixed, immunostained for actin (green) and vinculin (red), and photographed as described. The data described are representative of three independent experiments from three separate cell strains.

Cells plated in the absence of β-AR antagonist are polarized and crescent shaped with a broad lamellipodium, characteristic of the migratory phenotype (Ridley et al. (2003) Science 302:1704-1709). In keratinocytes the majority of the actin fibers and FAs appear to be restricted to the lamellipodia. β-AR antagonist treatment appears to have no effect on cytoskeletal conformation. The cell morphology, actin cytoskeleton and the number, size and distribution of focal adhesions appear similar to untreated keratinocytes.

β-AR Antagonists have No Effect on Keratinocyte Proliferation

Keratinocyte proliferation behind the epithelial tongue may be essential for efficient human skin re-epithelialization. Accordingly, the effect of antagonist treatment on proliferation was examined.

5×10⁴ keratinocytes were plated per well in a 12 well plate in triplicate and allowed to settle and attach to the plate for 2 hours. Cells were incubated in the presence or absence of β-AR antagonist (20 μM), (control (∘), β-AR antagonist (□)). Cells were harvested and counted on days 2, 4, 6, and 8 as described. The data are representative of three independent experiments with at least three different corneal epithelial cell strains. Values plotted are means+/−SEM. β-AR antagonists appear to have no effect on keratinocyte proliferation in vitro (FIG. 9).

β-AR Antagonists Accelerate Skin Wound Re-Epithelialization

The effect of β-AR antagonists on human skin re-epithelialization was investigated in an ex-vivo wound healing model adapted to resemble “chronic” wound healing (Pullar et al. (2006) Faseb J 20:76-86). Human skin punch biopsies are cultured in medium containing a reduced percentage of serum (5%) to significantly delay the re-epithelialization of untreated human skin wounds.

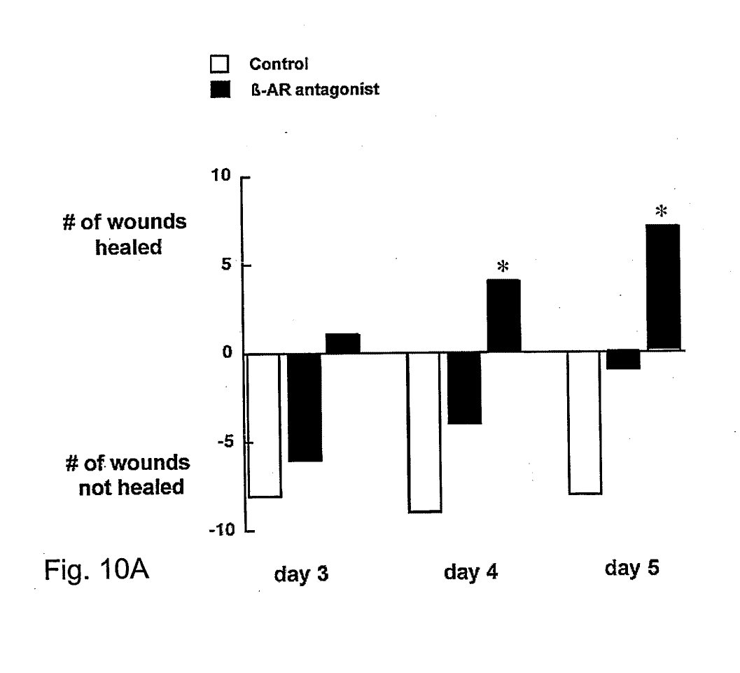

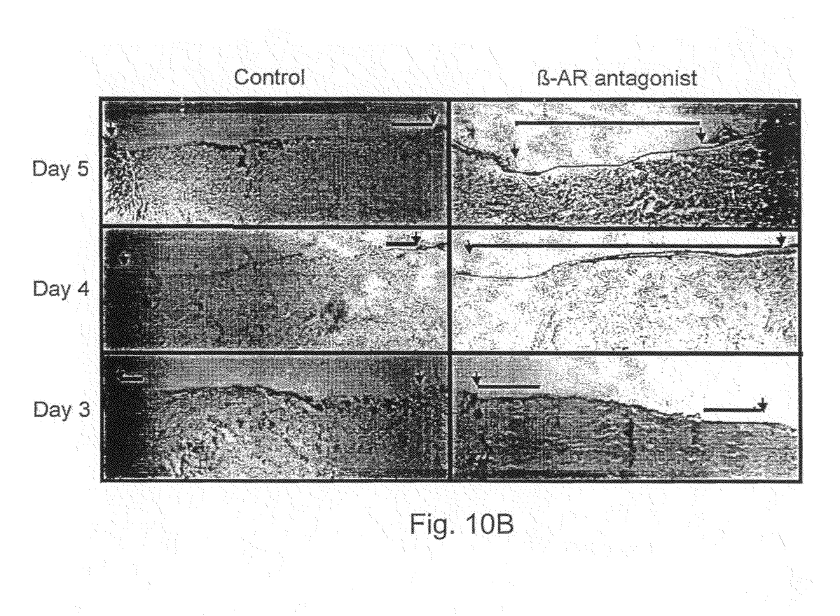

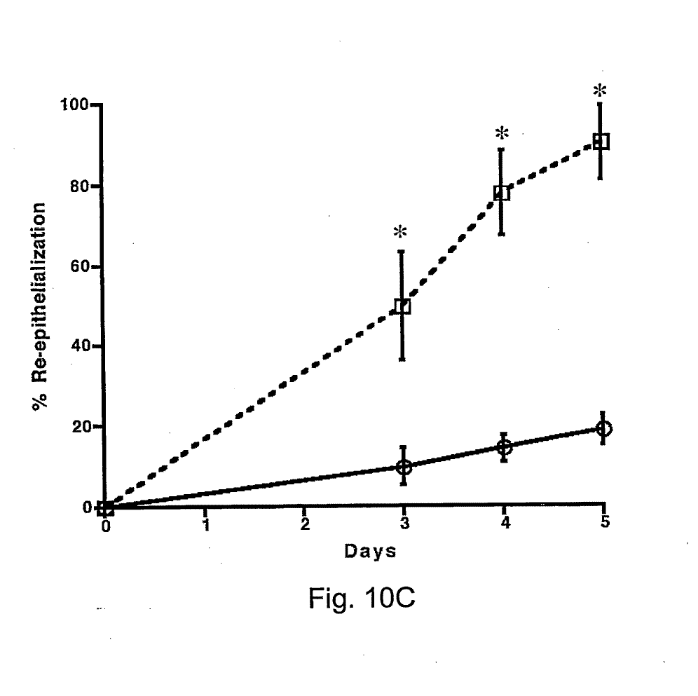

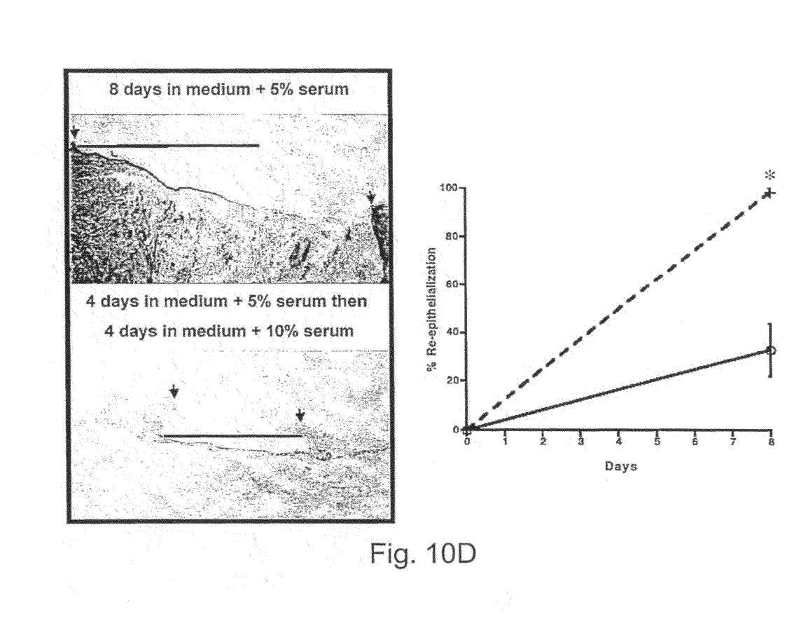

Wounds, 3 mm in diameter, were generated in excised human skin, cultured in medium containing 5% serum in the presence or absence of β-AR antagonist (10 μM), fixed and stained every day as described. Re-epithelialization was determined using light microscopy. A (+) score was given to a healed wound and a (−) score to any unhealed wounds. Specimens that were damaged in the histologic process or otherwise non interpretable were excluded from the study. Scores from experiments performed in triplicate on excised skin from 4 different individuals are graphically represented in FIG. 10 Panel A (* p<0.05, using the 2-tailed Fisher's exact test). Images of untreated (control) and β-AR antagonist-treated wounds, fixed on days 3-5, are presented in FIG. 10 Panel B at 10× magnification. The arrows mark the edges of the wound and the bars represent new epithelium. The % re-epithelialization was calculated for each wound, data was analyzed using the Student's t test and represented graphically, (control (∘), β-AR antagonist (□)) (FIG. 10 Panel C, *P<0.01). After 4 days of culture in medium containing 5% serum, biopsies were cultured for a further 4 days in the presence or absence of an additional 5% serum. Biopsies were fixed at day 8 and stained as described. Images of biopsies fixed and stained after 8 days in culture in 5% serum or 4 days in culture in 5% serum followed by 4 days in culture in 10% serum are presented in FIG. 10 Panel D at 10× magnification. The % re-epithelialization was calculated for each wound, data was analyzed using the Student's t test and represented graphically, (days 5-8 in 5% serum (∘______), days 5-8 in 10% serum (+ - - - -)) (FIG. 10 Panel D, *P<0.01). Data is combined from four independent experiments, performed in triplicate on excised skin from four different individuals.

Human skin was wounded and the wounds allowed to re-epithelialize in explant culture. No control wounds are healed within 5 days of wounding (FIG. 10 Panel A). In contrast, β-AR antagonist treatment significantly increases the rate healing of human skin wounds; one wound has completely re-epithelialized just 3 days post wounding, while 50% of the β-AR antagonist-treated wounds are completely re-epithelialized by day 4 and almost all β-AR antagonist-treated wounds are re-epithelialized by day 5 (FIG. 10 Panel A).

Hematoxylin and eosin-stained sections from control and β2-AR antagonist-treated wounds, days 3-5 are shown in FIG. 10 Panel B, highlighting the β-AR antagonist-mediated acceleration of skin wound repair. Due to variations in wound shape and the site within the wound from which sections were cut, leading to variation in the healing observed on days 3-5, the percentage of re-epithelialization was calculated for each wound. β-AR antagonist treatment significantly increases the wound re-epithelialization by 40%, 63% and 72% after 3, 4 and 5 days in culture, respectively (FIG. 10 Panel C). These results provide confirmation that β2-AR blockade accelerates wound reepithelialization in human skin.

To demonstrate that untreated wounds retained the capacity to heal, biopsies previously submerged in medium containing 5% serum for 4 days were cultured for a further 4 days in the presence or absence of an additional 5% serum prior to fixation. Wounds cultured in 10% serum for an additional 4 days were almost completely healed by day 8 (FIG. 10 Panel D). The % re-epithelialization of wounds cultured for the last 4 days in 10% serum was 66% higher than wounds cultured for the entire 8 day period in the presence of just 5% serum.

Keratinocytes Express the Enzymes Necessary to Convert L-Tyrosine to Epinephrine, Localized within Cytoplasmic Vesicles/Granules and Synthesize Epinephrine Endogenously

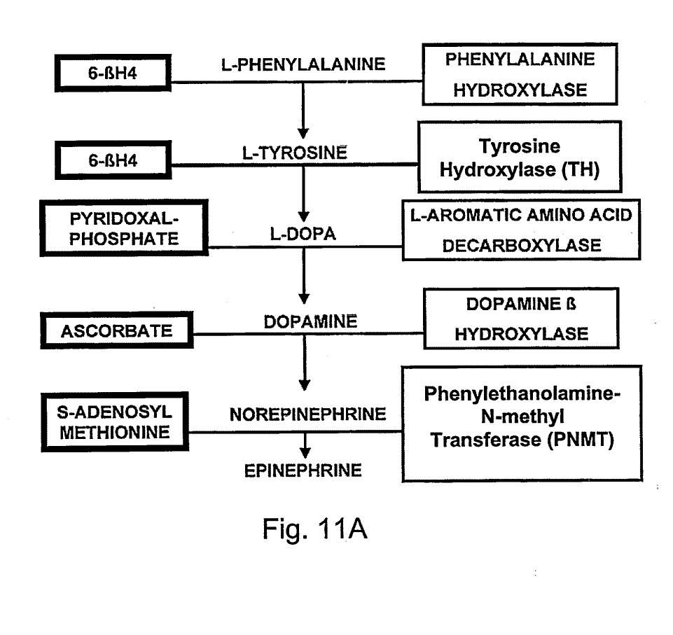

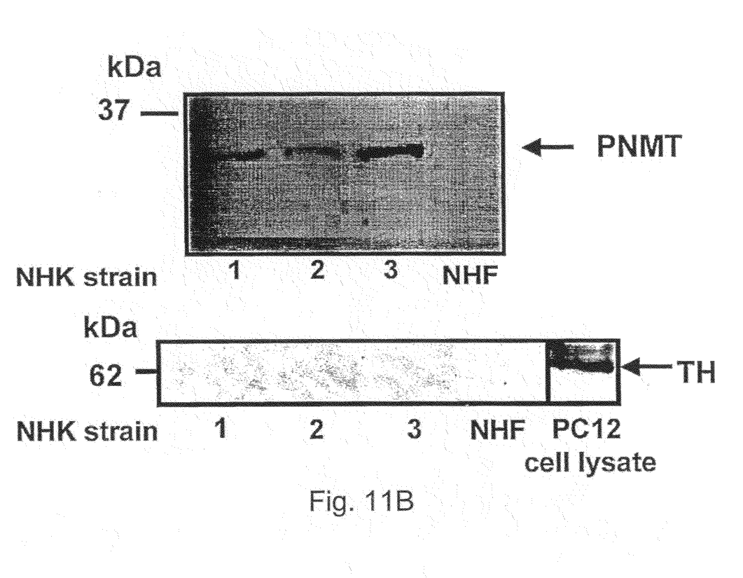

Catecholamines provide important biological functions, acting as both neurotransmitters and endocrine hormones. The conversion of L-tyrosine to L-dopa by tyrosine hydroxylase (TH) is the rate-limiting step for catecholamine biosynthesis (Nagatsu et al. (1964) Biochem Biophys Res Commun 14:543-549 and Nagatsu et al. (1964) J Biol Chem 239:2910-2917), and phenylethanolamine-N-methyl transferase (PNMT) catalyzes the synthesis of epinephrine from nor epinephrine (Schulz et al. (2004) Front Horm Res 31:1-25), as schematically illustrated in FIG. 11 Panel A. Previously, enzyme activity for TH and PNMT and mRNA for TH has been discovered in undifferentiated keratinocytes (Gillbro et al. (2004) J Invest Dermatol 123:346-353; Schallreuter et al. (1992) Biochem Biophys Res Commun 189:72-78; and Schallreuter et al. (1995) J Invest Dermatol 104:953-957). To determine if protein for catecholamine synthesis enzymes could be detected in keratinocytes, cells were lysed and immunoblotted with antibodies specific for TH and PNMT. A PC12 cell lysate was used as a positive control for TH (Nanmoku et al. (2005) J Endocrinol 186:233-239) but not for PNMT as PC12 cells contain negligible PNMT (Kano et al. (2005) Endocrinology 146:5332-40). A dermal fibroblast lysate was used as a negative control (Schallreuter et al. (1992) Biochem Biophys Res Commun 189:72-78). Human TH and PNMT enzymes are reported to be around 61-62 kDa and 30-32 kDas in size, respectively (Davidoff et al. (2005) Histochem Cell Biol:1-11). Indeed, the TH antibody detected one major protein at around 61 kDa and the PNMT antibody detected one major protein around 32 kDa (FIG. 11 Panel B).

To determine the localization of TH and PNMT in keratinocytes, cell cultures were immunostained with the anti-TH and anti-PNMT antibodies. Multiple brightly stained TH and PNMT-containing circular structures/granules can be observed distributed throughout the keratinocyte cytoplasm. Immunostaining was performed on dermal fibroblasts as a negative control (Schallreuter et al. (1992) Biochem Biophys Res Commun 189:72-78).

Finally, 303, 468 and 888 pg epinephrine per mg protein was measured in keratinocyte extracts from three different keratinocyte strains, as described. The detection of epinephrine in keratinocyte extracts provides convincing evidence that epinephrine is endogenously synthesized by keratinocytes.

β2-AR Antagonists Accelerate Skin Wound Healing

This example presents the novel finding that β-AR antagonists promote wound re-epithelialization, e.g., by blocking an autocrine β2-AR network within the epidermis. β-AR antagonists enhance the ability of keratinocytes to heal a scratch wound, increase the rate of single cell migration, increase ERK phosphorylation, enhance EF-mediated directional migration, preserve a pro-migratory cyto-architecture, maintain normal proliferation rates, and ultimately accelerate skin wound re-epithelialization. Keratinocytes express protein for two key enzymes in the catecholamine synthesis cascade, localized to cytoplasmic granules, and epinephrine can be measured in keratinocyte extracts. This is believed to be the first demonstration that β-AR antagonists can accelerate human skin wound re-epithelialization. Without intending to be limited to any particular mechanism, the mechanism of action by which the antagonists accelerate re-epithelialization can be via blockade of an endogenous autocrine β2-AR network that slows migration and delays wound healing; the β2-AR antagonist can block the binding of endogenous, keratinocyte-synthesized β-AR agonists (e.g., epinephrine and norepinephrine) to the receptor, thereby preventing any endogenous agonist-mediated decrease in ERK phosphorylation and migration. Antagonists can thus increase the phosphorylation of ERK and correspondingly increase keratinocyte migration and rate of wound repair.

ERK activation plays an important role in keratinocyte migration. Upon mechanical injury of confluent keratinocyte cultures (Turchi et al. (2002) J Invest Dermatol 119:56-63) or MDCK cultures (Matsubayashi et al. (2004) Curr Biol 14:731-735) ERK is activated by and is involved in wound repair in confluent rat keratinocyte cultures and in human epidermis (Providence and Higgins (2004) J Cell Physiol 200:297-308 and Stoll et al. (2002) J Biol Chem 277:26839-26845). Previously, it has been demonstrated that phospho-ERK is localized at the lamellipodial edge in migrating keratinocytes and β-AR agonists decrease ERK phosphorylation and prevent its localization to the lamellipodia via PP2A-dependent mechanisms (Pullar et al. (2006) Faseb 120:76-86 and Pullar et al. (2003) J Biol Chem 278:22555-22562). Although the function of phospho-ERK at the lamellipodial edge is not currently known, direct interactions between ERK and 13 integrins (Ahmed et al. (2002) Oncogene 21:1370-1380) or paxillin (Liu et al. (2002) J Biol Chem 277:10452-10458) may take place, suggesting an important role for ERK in integrating cell adhesion and receptor-mediated signaling in the control of cell migration. In contrast to the effects observed with β-AR agonists, β-AR antagonists enhance scratch wound healing in confluent keratinocyte cultures (FIG. 5), increase the rate of keratinocyte single cell migration (FIG. 6) and increase ERK phosphorylation (FIG. 7). As ERK plays such a pivotal role in cell migration, central to cutaneous wound repair, the β-AR antagonist-mediated increase in ERK phosphorylation and keratinocyte migration can optionally accelerate human skin wound re-epithelialization.

Wound currents have been measured exiting injured cornea and play a role in wound healing and limb regeneration in salamanders and newts (references above and Borgens et al. (1984) J Exp Zool 231:249-256; Borgens et al. (1977) Proc Natl Acad Sci U S A 74:4528-4532; and Altizer et al. (2002) J Exp Zool 293:467-477). EF application initiates epithelial cell cathodal migration within minutes and as an EF is generated immediately upon wounding, with the cathode at the wound center, it may be the earliest signal that epithelial cells receive to initiate directional migration into the dermal wound bed (references above and Robinson (1985) J Cell Biol 101:2023-2027 and Nuccitelli (2003) Curr Top Dev Biol 58:1-26). Previously, it has been demonstrated that PKA and the β2-AR-mediated increase in intracellular cAMP can modulate keratinocyte galvanotaxis (Pullar et al. (2001) Cell Motil Cytoskeleton 50:207-217 and Pullar and Isseroff (2005) J Cell Sci 118:2023-2034). This example demonstrates that a β-AR antagonist increases the ability of keratinocytes to sense and respond to the EF by exhibiting enhanced directional migration towards the cathode, an additional indication that β-AR antagonists enhance wound healing.

Efficient cell migration, typically required for wound repair, is dependent on temporally and spatially controlled reorganization of the actin cytoskeleton. Within hours of injury, keratinocytes undergo phenotypic alterations including the formation of a fine and diffuse actin network at the advancing lammellipodium to allow for cell migration (Gabbiani et al. (1978) J Cell Biol 76:561-568 and Kubler and Watt (1993) J Invest Dermatol 100:785-789). Integrin receptors within focal adhesions stabilize the lamellipodia (Frank and Carter (2004) J Cell Sci 117:1351-1363), allowing the migrating keratinocytes to interact with the variety of extra cellular matrices (ECMs) found in the wound site, including fibronectin, vitronectin, stromal type I collagen and fibrin (Larjava et al. (1993) J Clin Invest 92:1425-1435). β-AR antagonists preserve the pro-migratory phenotype of migrating keratinocytes; cells are polarized and crescent shaped with abroad lamellipodium, characteristic of the migratory phenotype. Keratinocyte proliferation behind the epithelial tongue is also central to efficient human skin re-epithelialization, and β-AR antagonists also preserve normal cell proliferation in vitro.

Strong evidence for the role of β2-AR in wound repair is the direct demonstration that blockade of β2-AR receptors in excised, wounded human skin (FIG. 10) significantly accelerates skin re-epithelialization. Using tissue confers the advantages of a normal ECM and the three dimensional geometry of the healing wound, not found in scratch assays or other assays using cultured cells.

Keratinocytes express a high level of β2-ARs, and enzyme activity for TH and PNMT and mRNA for TH has been discovered in undifferentiated keratinocytes. Interpretation of the enhanced keratinocyte migration and skin re-epithelialization observed in the presence of β-AR antagonists suggests that, without limitation to any particular mechanism, keratinocytes can synthesize and secrete catecholamines which are anti-motogenic and anti-mitogenic in keratinocytes and delay skin wound re-epithelialization. Blockade of the β2-AR can thus negate the endogenous catecholamine negative effects on keratinocyte migration, resulting in enhanced motility and wound healing. Immunoblots described above demonstrate that three different strains of keratinocytes express both enzymes, confirming that mRNA for these enzymes is indeed transcribed to protein. In addition, both catecholamine synthesis enzymes are localized to granules or vesicles in the keratinocyte cytoplasm. Finally, endogenously synthesized epinephrine was measured in keratinocyte extracts. Variation in the level of catecholamine synthesis enzymes expressed and epinephrine measured in the different strains of keratinocytes is observed.

β-AR antagonists are widely used drugs, for the treatment of cardiologic disease. Nearly 50 million Americans with hypertension are treated daily with β-AR antagonists (North Suburban Cardiology Group, Ltd. report (2001) “Facts About Hypertension”). They are also the most frequently prescribed class of drug for the treatment of glaucoma, a disease estimated to affect 1.25% of the population over 40 years of age and the leading cause of irreversible blindness in the world (Medeiros and Weinreb (2002) Drugs Today (Barc) 38:563-570 and Coleman and Brigatti (2001) Minerva Med 92:365-379). Elevated intraocular pressure is a major risk factor in glaucoma (Quigley (1996) Br J Opthalmol 80:389-393) and β-AR antagonists lower intraocular pressure (TOP), therefore minimizing damage to the optic nerve (Tan et al. (2002) J Glaucoma 11:134-142; Feldman (2004) Expert Opin Pharmacother 5:909-921; Sharif et al. (2001) J Ocul Pharmacol Ther 17:305-317; and Zimmerman (1993) J Ocul Pharmacol 9:373-384). Even though β-AR antagonists are used prolifically, to date there have been no specific observations regarding the ability of patients using these agents to heal wounds. The experiments described in this example demonstrate that β-AR antagonists significantly accelerate wound re-epithelialization.

### Example 3

### β-Adrenergic Receptor Agonists Delay while Antagonists Accelerate Epithelial Wound Healing

The following sets forth a series of experiments that demonstrate use of β2-AR agonists and antagonists to modulate the rate of re-epithelialization in corneal cell cultures and in corneal explants.

Corneal epithelial cells (CECs) must respond quickly to trauma to rapidly restore barrier function and protect the eye from noxious agents. CECs express a high level of β2-adrenergic receptors, but their function has not previously been reported. This example presents the novel finding that they form part of a regulatory network in the corneal epithelium, capable of modulating corneal epithelial wound repair. β-adrenergic receptor agonists delay corneal epithelial cell migration via a protein phosphatase 2A-mediated mechanism and decrease both electric field-directed migration and corneal wound healing. Conversely, β-adrenergic receptor antagonists accelerate corneal epithelial cell migration, enhance electric field-mediated directional migration, and promote corneal wound repair. CECs express key enzymes required for epinephrine (a β-adrenergic receptor agonist) synthesis in the cytoplasm, and epinephrine was detected in CEC extracts. Without intending to be limited to any particular mechanism, the mechanism for the pro-motogenic effect of the β-adrenergic antagonist can be blockade of the β2-adrenergic receptor, preventing autocrine catecholamine binding.

Visualizing cell migration within tissue confers the advantage of observing cell behaviors within their natural three-dimensional environment. Cornea was chosen as a model system within which to study epithelial wound healing due to its transparency, allowing individual cells to be viewed directly with high spatial and temporal resolution (Zhao et al. (2003) “Direct visualization of a stratified epithelium reveals that wounds heal by unified sliding of cell sheets” Faseb J 17:397-406).

One of the most important functions of the cornea is to maintain normal vision by transmitting light onto the lens and retina, while protecting the eye from both physical trauma and harmful environmental agents. To enable the cornea to perform this duty, the epithelium is continuously renewed to maintain its smooth optical properties and barrier function. Corneal epithelial cells (CECs) must respond quickly to trauma to restore sight and minimize infection. Upon injury, cells migrate directionally towards the center of the wound bed to initiate repair and restore epithelial integrity. Many cues play a role in wound-induced CEC directional migration including contact inhibition, chemotaxis (Lu et al. (2001) “Corneal epithelial wound healing” Exp Biol Med (Maywood) 226:653-64 and Wilson et al. (2001) “The corneal wound healing response: cytokine-mediated interaction of the epithelium, stroma, and inflammatory cells” Prog Retin Eye Res 20:625-37) and galvanotaxis. CECs migrate directionally towards the cathode of an applied direct current (DC) electric field (EF) (Farboud et al. (2000) “DC electric fields induce rapid directional migration in cultured human corneal epithelial cells” Exp Eye Res 70:667-73 and Zhao et al. (1997) “Human corneal epithelial cells reorient and migrate cathodally in a small applied electric field” Curr Eye Res 16:973-84), and electric currents play an important role in corneal wound healing (Song et al. (2002) “Electrical cues regulate the orientation and frequency of cell division and the rate of wound healing in vivo” Proc Natl Acad Sci USA 99:13577-82 and Sta Iglesia and Vanable (1998) “Endogenous lateral electric fields around bovine corneal lesions are necessary for and can enhance normal rates of wound healing” Wound Repair Regen 6:531-42). Indeed, increasing or decreasing corneal wound currents pharmacologically can correlate directly with an increased or decreased rate of healing in the rat cornea (Reid et al. (2005) “Wound healing in rat cornea: the role of electric currents” Faseb J 19:379-86).

β-ARs are expressed widely in a variety of tissues and are recognized as pivotal regulators of the cardiac, pulmonary, vascular, endocrine and central nervous systems. CECs express a high level of β2-ARs (Elena et al. (1990) “Beta adrenergic binding sites in the human eye: an autoradiographic study” J Ocul Pharmacol 6:143-9; Kahlee et al. (1990) “Quantitative autoradiography of beta-adrenergic receptors in rabbit eyes” Exp Eye Res 51:503-7; and Walkenbach et al. (1984) “Characteristics of beta-adrenergic receptors in bovine corneal epithelium: comparison of fresh tissue and cultured cells Biochem Biophys Res Commun 121:664-72). Sympathetic nerves terminating in the cornea release catecholamines, known to play a role in corneal homeostasis and wound healing (Friedenwald and Buschke (1944) “Some factors concerned in the mitotic and wound-healing activities of the corneal epithelium” Transcripts of the American Opthalmology Society 42:371 and Perez et al. (1987) “Effects of chronic sympathetic stimulation on corneal wound healing” Invest Opthalmol Vis Sci 28:221-4). Indeed, catecholamines can be detected in lacrimal secretions from healthy volunteers (Trope and Rumley (1984) “Catecholamine concentrations in tears” Exp Eye Res 39:247-50 and Zubareva and Kiseleva (1977) “Catecholamine content of the lacrimal fluid of healthy people and glaucoma patients” Opthalmologica 175:339-44). Historically, the reports of the effect of β-AR antagonists on corneal epithelial wound healing have been conflicting and no mechanisms of action have been proposed. Some studies report a β-AR antagonist-mediated delay in rabbit (Haruta et al. (1997) “Corneal epithelial deficiency induced by the use of beta-blocker eye drops” Eur J Opthalmol 7:334-9; Liu et al. (1990) “Beta adrenoceptors and regenerating corneal epithelium” J Ocul Pharmacol 6:101-12; and Nork et al. (1984) “Timolol inhibits corneal epithelial wound healing in rabbits and monkeys” Arch Opthalmol 102:1224-8), while other studies report a β-AR antagonist mediated acceleration of wound closure in rabbit corneas (Reidy et al. (1994) “Effect of topical beta blockers on corneal epithelial wound healing in the rabbit” Br J Ophthalmol 78:377-80). Additionally, norepinephrine can increase the rate of human corneal epithelial cell migration (Murphy et al. (1998) “Effect of norepinephrine on proliferation, migration, and adhesion of SV-40 transformed human corneal epithelial cells” Cornea 17:529-36) and corneal wound repair.

The experiments described in this example investigate the effect of β-AR activation or blockade on the corneal wound healing process. They demonstrate that β-AR activation decreases the rate of CEC single cell migration via a protein phosphatase 2A (PP2A)-mediated mechanism, partially blinds cells to an applied EF, and ultimately delays corneal wound healing. In contrast, β-AR antagonists increase extracellular signal-related kinase (ERK) phosphorylation, enhance scratch wound healing, increase the rate of CEC single cell migration, enhance the ability of cells to migrate directionally in an applied EF, and ultimately accelerate corneal wound healing. Delineation of the pro-migratory mechanisms activated by β-AR antagonists revealed the novel finding that CECs express key enzymes required for catecholamine synthesis, localized to cytoplasmic vesicles, and synthesize epinephrine. This work uncovers a previously unrecognized endogenous β-AR regulatory network in the cornea, capable of modulating corneal wound repair.

### Materials and Methods

Materials

Materials were purchased as follows: Isoproterenol (β-AR agonist) and okadaic acid (Calbiochem (San Diego, Calif.)), timolol (β-AR antagonist) and the anti-vinculin antibody (h-vin-1) (Sigma (St. Louis, Mo.)), Anti-ERK (#9102) and anti-phospho-ERK (#9101) antibodies (Cell Signaling Technology (Beverly, Mass.)), the anti-tyrosine hydroxylase antibody (TH, AB152) (Chemicon (Temecula, Calif.)), and the anti-phenylethanolamine-N-methyl transferase antibody (PNMT) (Biogenesis (Brentwood,

Corneal Epithelial Cell Growth

Human adult corneas that had been donated for research were obtained from the Sierra Eye and Tissue Donor Services (Sacramento, Calif., a regional center of Dialysis Clinics Inc., Donor Services, Nashville, Tenn.) within 2-14 days of collection. Corneas were stored in Optisol-GS corneal storage medium (Chiron Ophthalmics, Irvine, Calif.) at 2-8° C., and were transported to the laboratory on ice. The research followed the tenets of the Declaration of Helsinki; tissue was obtained with appropriate consents from either donor or next of kin and was approved by the University of California, Davis Institutional Review Board (IRB). CECs were isolated as previously described (Farboud et al., supra), and maintained in a 37° C. incubator with 5% CO₂ in corneal growth medium (CGM) consisting of: EpiLife medium, supplemented with 0.18 μg/ml hydrocortisone, 5 μg/ml transferrin, 5 μg/ml insulin, 0.2% bovine pituitary extract and 1 ng/ml mouse EGF, calcium (final concentration 0.06 mM) (Cascade Biologics, Inc., Portland, Oreg.) and antibiotics/antimycotic (100 units penicillin G per ml, 100 μg streptomycin per ml, and 0.25 μg amphotericin B per ml (Gibco, Grand Island, N.Y.). Passage 3-7 cells were used for all experiments.

Bovine Corneal Epithelial Cell Isolation

The culture of primary bovine CECs has been described in detail elsewhere (Zhao et al. (1996b) “Orientation and directed migration of cultured corneal epithelial cells in small electric fields are serum dependent” J Cell Sci 109 (Pt 6):1405-14). Here; the method has been modified slightly by use of a specialized medium (EP) in which the cells were initially cultured. EP consists of a 3:1 ratio by volume of Ham's F12 nutrient mixture containing L-glutamine and Dulbecco's modified Eagle's medium (DMEM) without L-glutamine; 2.5% fetal calf serum; 0.4 μg/ml hydrocortisone; 8.4 ng/ml cholera toxin; 5 μg/ml insulin (Sigma, St. Louis, Mo.); 24 μg/ml adenine; 10 ng/ml EGF, and antibiotics (100 units penicillin G per ml, 100 μg streptomycin per nil) (Gibco, Grand Island, N.Y.).

Scratch Assay

Adult human CECs were grown to confluence in CGM on plastic tissue culture dishes (Fisher Scientific, Pittsburgh, Pa.) coated with 60 μg/ml collagen I (Vitrogen 100, Collagen Corp., Palo Alto, Calif.) in PBS for 1 hour at 37° C. A sterile pipette tip was used to scratch a 1 mm-wide wound along the center of the dish and the media was replaced with either CGM alone or CGM containing 20 μM β-AR antagonist. A demarcated area of the wound was photographed on an inverted Nikon Diaphot microscope at the time of wounding (time 0) up to wound healing as described (Pullar et al. (2003) “PP2A activation by beta2-adrenergic receptor agonists: novel regulatory mechanism of keratinocyte migration” J Biol Chem 278:22555-62).

Cell Treatments for Immunoblotting

1×10⁶ plated adult human CECs were incubated with either CGM alone (control and lysates for catecholamine synthesis enzyme detection) or CGM containing 20 μM β-AR antagonist for 5-60 minutes. Lysates were prepared as described (Pullar et al. (1996) “Rapid dephosphorylation of the GTPase dynamin after FcepsilonRI aggregation in a rat mast cell line” J Immunol 157:1226-32). The protein concentration of the samples was determined using the Bradford Assay (Bio-Rad Laboratories, Hercules, Calif.). The supernatants were electrophoresed immediately on 10% polyacrylamide Bis-Tris gels (Bio-Rad Laboratories, Hercules, Calif.) or stored at −80° C. Five μg (P-ERK blots) or thirty-five μg (catecholamine synthesis enzyme blots) of each protein sample was added to an equal volume of 2× reducing sample loading buffer (0.0625M Tris-HCl pH 6.8, 3% SDS, 10% glycerol, 5% β-ME) and electrophoresed on 10% polyacrylamide Bis-Tris gels. Proteins were transferred to Immobilon membranes and immunoblotted with an anti-ERK (#9102), phospho-ERK (#9101), TH or PNMT antibody at a concentration recommended by the manufacturer. The immunoblots were developed by enhanced chemiluminescence (ECL) according to the manufacturer's instructions (Amersham Pharmacia Biotech, Piscataway, N.J.). Densitometry was performed on scanned images using NIH Image 1.62.

Single Cell Migration Assay

Glass bottomed 35 mm dishes (MatTek Corporation, Ashland, Mass.) were coated with collagen I (60 μg/ml) (Cohesion Technologies, Palo Alto, Calif.) in PBS for 1 hour at 37° C. Adult human CECs were plated at a density of 50 cells/mm² for 2 hours at 37° C. Cells were pre-incubated with either CGM alone or CGM containing 100 nM okadaic acid (OA) for 45 minutes at 37° C. Untreated cells were then incubated with CGM alone (control) or with CGM containing either 20 μM β-AR antagonist, 10 nM or 1 μM β-AR agonist. Pre-treated cells were stimulated with 100 nM OA alone or both 10 nM or 1 μM β-AR agonist and 100 nM OA at time 0. The 35 mm glass-bottomed dishes were placed in a heating chamber, designed to maintain the media between 35-37° C., secured to the stage of an inverted Nikon Diaphot microscope. Individual cell migration was monitored over a 1 hour period at 37° C., as described previously (Pullar and Isseroff (2005b) “Cyclic AMP mediates keratinocyte directional migration in an electric field” J Cell Sci 118:2023-2034). Time-lapse images of the cell migratory response were digitally captured every 10 minutes by Q-Imaging Retiga-EX cameras (Burnaby, BC, Canada) controlled by a custom automation written in Improvision Open Lab software (Lexington, Mass.) on a Macintosh G4. After each cell's center of mass was tracked using the Open Lab software, migration speed and distance were calculated and imported to Excel (Microsoft Corporation, Redmond, Wash.). Significance was taken as P<0.01, using Student's t test (unpaired) to compare the means of two cell populations.

Immunofluorescent Staining and Microscopy

Sterile glass cover slips were transferred into 12 well dishes and collagen-coated with 60 μg/ml collagen I in PBS for 1 hour at 37° C. Cover slips were washed three times with CGM and 3×10⁴ adult human CECs were added per well and allowed to attach for 3 hours or overnight. Cells were untreated (control and catecholamine synthesis enzymes), treated with 20 μM β-AR antagonist, 10 nM or 1 μM β-AR agonist for 15 minutes, 100 nM OA for 45 minutes, or pre-treated with 100 nM OA for 30 minutes prior to the addition of 10 nM or 1 μM β-AR agonist for 15 minutes. Cover slips were processed at room temperature unless otherwise noted and immunofluorescent staining was performed as previously described (Pullar and Isseroff (2006) “β2-adrenergic receptor activates pro-migratory and pro-proliferative pathways in dermal fibroblasts via divergent mechanisms” Journal of Cell Science 119:592-602). Slides were viewed on an inverted fluorescent Nikon Diaphot microscope using a 40× pan fluor objective. Images were captured using Q-imaging Retiga-EX cameras (Burnaby, BC, Canada) and pseudo-colored green for Alexa 488 Phalloidin staining (actin) or red for Cy3 staining (vinculin, TH, PNMT) using Improvision Openlab software (Lexington, Mass.).

Galvanotaxis

Primary bovine CECs were seeded at low density in EP medium within electrotactic chambers resting on tissue culture dishes, for 2-3 hours prior to EF exposure. A roof consisting of a No 1 cover slip was applied and sealed on top of the chamber, as previously described (Zhao et al. (1996a) “Directed migration of corneal epithelial sheets in physiological electric fields” Invest Opthalmol Vis Sci 37:2548-58). The final dimensions of the chamber through which the electric current was passed were 40 mm×10 mm×0.3 mm. A direct current EF of 50 mV/mm was applied through agar-salt bridges connecting silver/silver chloride electrodes via beakers of Steinberg's solution, to pools of culture medium at either side of the chamber. The dish was placed on a Zeiss Axiovert 100 microscope with temperature control at 37° C.

Time-Lapse Video Microscopy and Quantification of Cell Migration

Time-lapse images were recorded every 5 minutes and analyzed with a MetaMorph system (Universal Imaging Corporation, PA) (Zhao et al. (2002) “Membrane lipids, EGF receptors, and intracellular signals colocalize and are polarized in epithelial cells moving directionally in a physiological electric field” Faseb J 16:857-9). Migration directedness (cosine θ shows how directionally a cell migrated within the field, where θ is the angle between the EF vector and a straight line connecting the start and end position of a cell (Zhao et al. (1996a) supra). A cell moving perfectly toward the cathode would have a directedness of 1; a cell moving perfectly along the field lines toward the anode would have a directedness of −1. Therefore, the average of directedness values of a population of cells gives an objective quantification of how directionally cells have moved. A group of cells migrating randomly would have an average directedness value of 0. Migration rate was analyzed with the following 2 parameters. Trajectory speed (Tt/T) is the total length of the migration trajectory of a cell (Tt) divided by the given period of time (T). Displacement speed (Td/T) is the straight-line distance between the start and end positions of a cell (Td) divided by the time (T).

Proliferation Assay

Adult human CECs were released from the tissue culture plate by treatment with 0.25% trypsin/0.1% EDTA (Gibco, Grand Island, N.Y.), resuspended in CGM and counted using a hemocytometer. 5×10⁴ cells were plated per well in a 12 well plate in triplicate and allowed to settle and attach to the plate for 2 hours prior to β-AR agonist (10 nM, 1 μM) or antagonist (20 μM) addition. Cells were then cultured in the presence or absence of 10 nM β-AR agonist or 20 μM β-AR antagonist for 8 days with media changes every day. Triplicate wells were harvested and counted on days 2, 4, 6, 8. The means of the two cell populations were compared using Student's t test (unpaired).

Bovine Corneal Ex-Vivo Wound Healing Assay

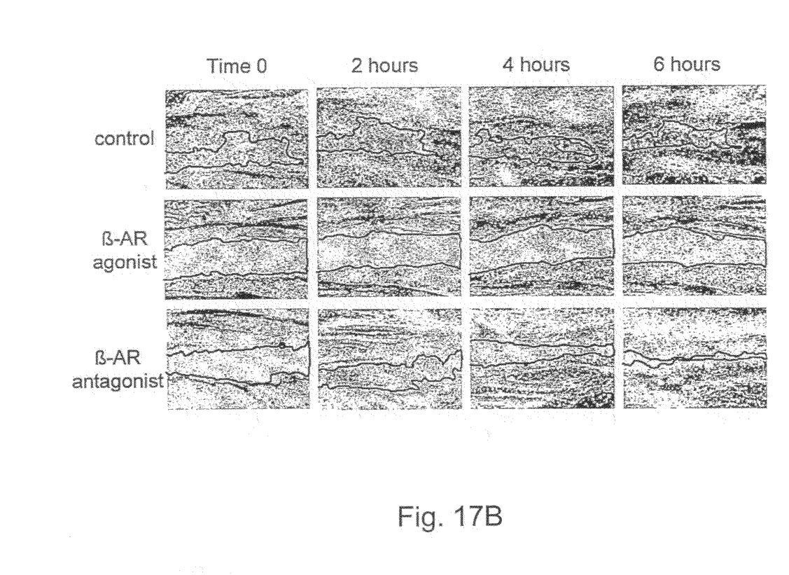

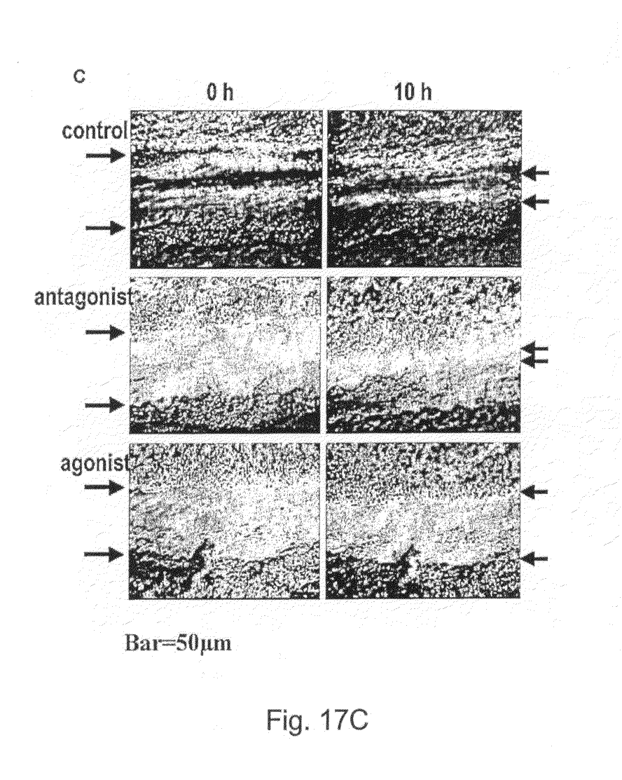

Bovine eyes were obtained from McIntosh. Donald Ltd, Portlethan, UK and used within a few hours of harvest. Five eyes were used per treatment group. Bovine eyes were secured in a specially designed chamber, placed under a Motic dissecting microscope and a linear wound 200-300 μM wide was created using an ophthalmic surgical blade (Medical Sterile Products, Rincon, Puerto Rico). The corneas were surgically removed from the eye using a sterile scalpel blade and immediately transferred to either a sterile 6 well tissue culture dish (FIG. 17 Panels A and B) or a 60 mm tissue culture dish and submerged in 4 ml of media (1:1 EP:CO₂-independent medium) containing antibiotics and 10% fetal bovine serum in the presence or absence of either 10 μM β-AR agonist or β-AR antagonist. The 6 well dishes were incubated at 37° C. in a humidified atmosphere of 5% CO₂. Corneal wounds were visualized on an inverted Nikon Diaphot 300 microscope using a 20× objective at time 0 and 2, 4 and 6 hours post wounding. Images were captured with a Sony XC-75CE CCD video camera using Leica QWin software at 3 different places along each incisional wound. Image J was used to measure the wound area at time 0 and subsequent time points post wounding to calculate the % healing. Significance was taken as P<0.01, using Student's t test. The 60 mm dishes were placed on a Zeiss Axiovert 100 microscope with temperature control at 37° C. Time-lapse images were recorded every 2 minutes up to 10 hours on a MetaMorph imaging system and the images were compiled into a movie using Quick time software.

Enzyme Immunoassay for the Quantitative Determination of Epinephrine in Small Sample Volumes

1×10⁷ keratinocytes were extracted in 100 μl 0.1NHCl and sonicated on ice for 10 minutes. Extracts from three strains of keratinocytes were tested in triplicate in an epinephrine enzyme immunoassay (EIA) (Biosource, Camarillo, Calif.) according to the manufacturers instructions. Briefly, the assay kit provides materials for the quantitative measurement of epinephrine. Epinephrine is extracted using a cis-diol specific affinity gel, then acylated to N-acylepinephrine and after this converted enzymatically during the detection procedure into N-acylmetanephrine. The competitive EIA uses the microliter plate format. Epinephrine is bound to the solid phase of the microliter plate. Acylated epinephrine and solid phase bound epinephrine compete for a fixed number of antiserum binding sites. When the system is in equilibrium, free antigen and free antigen-antiserum complexes are removed by washing. The antibody bound to the solid phase catecholamine is detected by an anti-rabbit IgG peroxidase conjugate using TMB as a substrate. The reaction is monitored at 450 nm on a Spectramax 340PC spectrophotometer (Molecular Devices Corp., Sunnyvale, Calif.) with the amount of antibody bound to the solid phase being inversely proportional to the catecholamine concentration in the sample. A set of standard and two controls are included in the EIA kit for determination of unknown concentrations (0, 5.6, 19, 83, 306, 1550 pg epinephrine/sample). The linear mean absorbance readings of the standards are plotted on the y-axis versus the log of the concentrations of the standards (pg/sample) on the x-axis and a linear curve fit is applied. The concentration of epinephrine in the unknowns can then be calculated from the slope of the line. The protein concentration in each extract is calculated using the Bradford assay, as previously described, to standardize the amount of epinephrine measured per mg of protein in the extract.

Corneal Wound Healing Movie

Bovine corneas were wounded and placed into 60 mm dishes in the absence or presence of 10 μM β-AR agonist or 10 μM β-AR antagonist as described. The 60 mm dishes were placed on a Zeiss Axiovert 100 microscope with temperature control at 37° C. Time-lapse images were recorded every 2 minutes up to 10 hours on a MetaMorph imaging system and the images were compiled into a movie using Quick time software at a display rate of 60 frames/second. The time 0 and 10 hour frames of the corneas are displayed in FIG. 17 Panel C. Four corneas were wounded and imaged per group.

### Results

A β-AR Antagonist Accelerates the Healing of Scratch Wounds in Confluent Adult Human Corneal Epithelial Cell Cultures

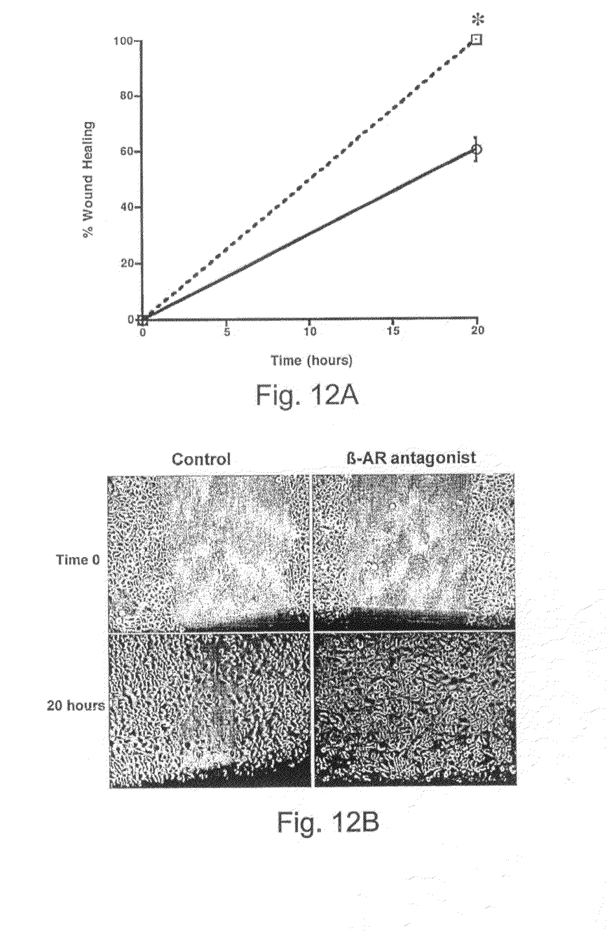

The “scratch” assay is a useful in vitro model of wound healing. A denuded area is created within a confluent sheet of cells and the healing of the “wound” can be observed microscopically and quantified by calculating the percentage of healing over time (Pullar et al. (2003) supra). Adult human CECs were grown to confluence on collagen-coated plastic dishes as described. Cultures were wounded and CGM alone or CGM containing 20 μM timolol was added at Time 0. A demarcated area of the wound was photographed at the time of wounding (time 0) and again at 20 hours. The % wound healing was calculated and is represented graphically in FIG. 12 Panel A, control (◯______) timolol ( [symbol] --------). Images of control and β-AR antagonist-treated wounds at time 0 and 20 hours after wounding are represented in FIG. 12 Panel B. The data shown are representative of three independent experiments from three separate cell strains. Values plotted are means+/−SEM (n=3). * P<0.01 between β-AR antagonist and control.

As shown in FIG. 12, the β-AR antagonist significantly accelerates human corneal epithelial scratch wound healing. Untreated wounds are only 60% healed within 20 hours, whereas β-AR antagonist-treated wounds are completely healed within the same time frame.

A β-AR Antagonist Increases ERK Phosphorylation within Minutes in Adult Human Corneal Epithelial Cells

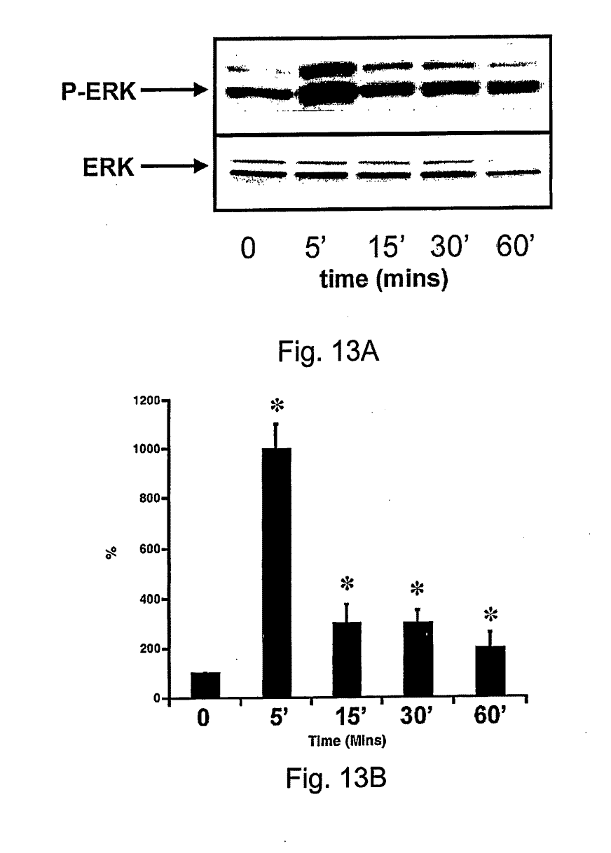

ERK plays a pivotal role in pro-migratory signaling pathways (Glading et al. (2000) “Epidermal growth factor receptor activation of calpain is required for fibroblast motility and occurs via an ERK/MAP kinase signaling pathway” J Biol Chem 275:2390-8 and Zeigler et al. (1999) “Role of ERK and JNK pathways in regulating cell motility and matrix metalloproteinase 9 production in growth factor-stimulated human epidermal keratinocytes” J Cell Physiol 180:271-84), is critical for the healing of scratch wounds in confluent monolayers of lens and corneal epithelial cells, and is phosphorylated within an hour of rat corneal wounding (Shanley et al. (2004) “Insulin, not leptin, promotes in vitro cell migration to heal monolayer wounds in human corneal epithelium” Invest Opthalmol Vis Sci 45:1088-94; Wang et al. (2003) “Electric fields and MAP kinase signaling can regulate early wound healing in lens epithelium” Invest Opthalmol Vis Sci 44:244-9; Xu et al. (2004) “Role of ErbB2 in Corneal Epithelial Wound Healing” Invest Opthalmol Vis Sci 45:4277-83 and Imayasu and Shimada (2003) “Phosphorylation of MAP kinase in corneal epithelial cells during wound healing” Curr Eye Res 27:133-41). Concurrent with the β-AR antagonist-mediated increase in corneal epithelial scratch wound healing, β-AR antagonist treatment dramatically increases ERK phosphorylation by 10-fold within 5 minutes. ERK phosphorylation remains elevated for up to 60 minutes in the presence of β-AR antagonist while gradually returning towards control levels (FIG. 13 Panels A and B).

CECs were cultured in CGM and plated as described. Cells were either untreated (time 0) or incubated with 20 μM β-AR antagonist for up to 60 minutes at 37° C. After treatment, cell lysates were prepared as described, electrophoresed on 10% polyacrylamide gels and transferred to membrane. Identical membranes were immunoblotted with either an anti-phospho ERK (P-ERK) or an anti-ERK antibody (FIG. 13 Panel A). The data shown are representative of three independent experiments from three separate cell strains. Three blots from separate experiments were scanned for ERK and P-ERK and densitometry performed using a gel plotting macro in NIH Image 1.62. Data was normalized, averaged, statistically analyzed and represented graphically (FIG. 13 Panel B). Values plotted are means±SEM (n=3). * P<0.01 compared to the control.

A β-AR Agonist Decreases the Rate of Adult Human Corneal Epithelial Single Cell Migration Via a PP2A-Dependent Mechanism while Conversely a β-AR Antagonist Enhances the Migration Rate

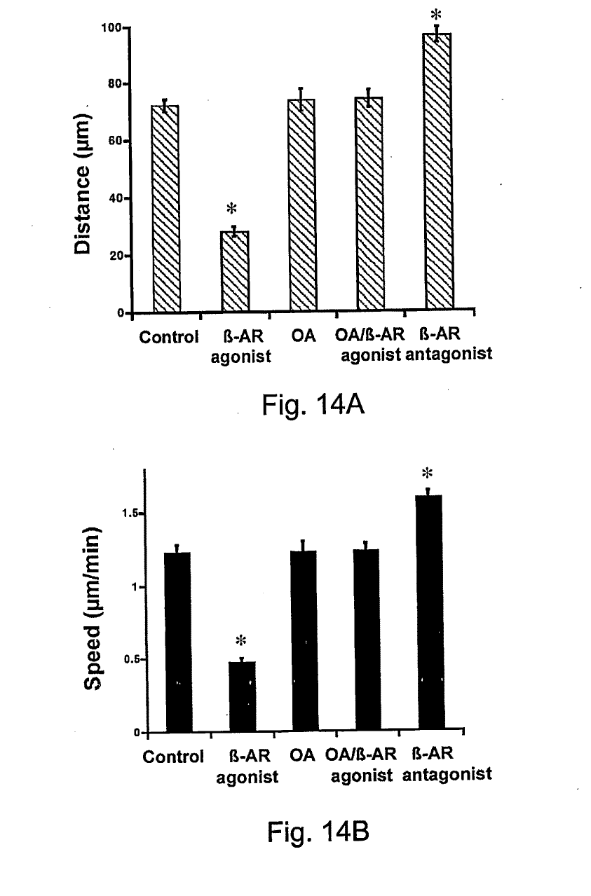

To more precisely measure the effect of β-AR ligands on motility, the effect of β2-AR activation and blockade on the locomotory speed of individual adult human CECs (Pullar et al. (2003) supra) was observed. CECs were pre-treated with OA (100 nM, OA, OA/β-AR agonist) for 30-45 minutes at 37° C. or not. The medium was replaced with CGM (control), CGM containing 20 μM β-AR antagonist, 10 nM β-AR agonist, 100 nM OA, or both OA and agonist at time 0 and the migration of each single cell monitored over a one-hour period, as described. The distance traveled in 1 hour and the speed of migration are represented graphically in FIG. 14 Panels A and B, respectively (n=215 (control), n=210 (β-AR agonist), n=96 (OA), n=120 (OA/β-AR agonist) n=189 (β-AR antagonist)). The data shown are representative of multiple independent experiments from five separate cell strains. Values plotted are means+/−SEM. * P<0.01 between agonist or antagonist and control.

β-AR agonist reduces the rate of corneal epithelial single cell migration by 62% (FIG. 14 Panels A and B). Increasing the concentration of β-AR agonist to 1 μM reduces the cell migration rate even further. Pre-treatment with okadaic acid (OA) alone, a PP2A inhibitor (Bialojan and Takai (1988) “Inhibitory effect of a marine-sponge toxin, okadaic acid, on protein phosphatases” Specificity and kinetics Biochem J 256:283-90; Fernandez et al. (2002) “Okadaic acid, useful tool for studying cellular processes” Curr Med Chem 9:229-62; and Millward et al. (1999) “Regulation of protein kinase cascades by protein phosphatase 2A” Trends Biochem Sci 24:186-91), has no effect on migration rate. However, it completely prevents the β-AR agonist-mediated decrease in the rate of corneal epithelial single cell migration, demonstrating that the mechanism for the β-AR-mediated anti-motogenicity is PP2A-dependent. In contrast, a β-AR antagonist significantly increases the rate of CEC migration by 34% (FIG. 14 Panels A and B).

A β-AR Agonist Alters the Cytoskeletal Conformation of Adult Human Corneal Epithelial Cells Via a PP2A-Dependent Mechanism, while a β-AR Antagonist Preserves the Pro-Migratory Cytoskeletal Architecture

Efficient cell migration, required for wound repair, is dependent on the temporally and spatially controlled reorganization of the actin cytoskeleton (Pantaloni et al. (2001) “Mechanism of actin-based motility” Science 292:1502-6). Actin filaments terminate in focal adhesions (FAs), where several proteins, including vinculin, mediate interactions with the actin cytoskeleton and play a role in cell migration (Burridge and Fath (1989) “Focal contacts: transmembrane links between the extracellular matrix and the cytoskeleton” Bioessays 10:104-8 and Beningo et al. (2001) “Nascent focal adhesions are responsible for the generation of strong propulsive forces in migrating fibroblasts” J Cell Biol 153:881-8). Since β2-AR activation decreases the rate of CEC migration, whether these effects involve alterations in the actin cytoskeleton was examined.

Sterile cover slips were coated with collagen and cells plated as described. Cells were left untreated, treated with β-AR agonist (10 nM) or β-AR antagonist (20 μM) for 15 minutes, treated with OA (100 nM) for 45 minutes or pre-treated with OA (100 nM) for 30 minutes prior to the addition of both OA (100 nM) and β-AR agonist (10 nM) for 15 minutes. Cells were fixed, immunostained for actin (green) and vinculin (red) and photographed as described. The data described are representative of three independent experiments from three separate cell strains.

Cells plated in the absence of β-AR agonist are polarized and crescent shaped with a broad lamellipodium, characteristic of the migratory phenotype (Ridley et al. (2003) “Cell migration: integrating signals from front to back” Science 302:1704-9). In untreated CECs the majority of the actin fibers and FAs appear to be restricted to the lamellipodia. Pre-treating with a β-AR agonist for 15 minutes markedly alters the CEC morphology. Cells are now rounded with no apparent polarization. Cortical actin stress fibers are localized around the internal borders of the cell and vinculin-rich FAs are distributed evenly around the cell periphery. Image J was used to measure the actin and vinculin associated fluorescence by measuring the mean pixel intensity (MPI) of 25 cells from each condition. Control cells have an MPI of 40.3+/−4.8. β-AR agonist treatment has no significant effect on actin and vinculin associated fluorescence; the MPI of β-AR agonist-treated cells is 44.6+/−2. Similar results were obtained with higher concentrations of β-AR agonist (1 μM).

To determine if the β-AR agonist-mediated alteration in the cyto-architecture of actin stress fibers and vinculin-rich FAs is also mediated by PP2A, CECs were pre-treated with the PP2A-specific inhibitor, OA, prior to exposure to β-AR agonist. OA treatment alone has no effect on the cytoskeletal organization, with cells displaying a normal migratory phenotype. There was also no significant difference in actin and vinculin associated fluorescence; the MPI is 43.6+/−5. However, pre-treating CECs with OA prior to adding β-AR agonist prevents the β2-AR-mediated alterations in cytoskeletal organization. The actin and vinculin associated fluorescence of OA-β-AR agonist-treated cells is similar to untreated cells with an MPI of 38.7+/−4.6. OA pre-treatment restores the migratory phenotype observed in untreated CECs, confirming that the mechanism for the observed β2-AR-mediated cytoskeletal re-organization is PP2A-dependent.

In contrast, β-AR antagonist treatment has no effect on cytoskeletal conformation. The cell morphology, actin cytoskeleton and the number, size and distribution of focal adhesions appear similar to untreated CECs. The MPI of the actin and vinculin associated fluorescence was similar to control cells, 41.2+/−5.

A β-AR Agonist Decreases the Ability of Bovine Corneal Epithelial Cells to Migrate Cathodally in an Applied Electric Field, while a β-AR Antagonist Enhances Both Bovine Corneal Epithelial Cell EF-Mediated Directionality and Rate of Migration

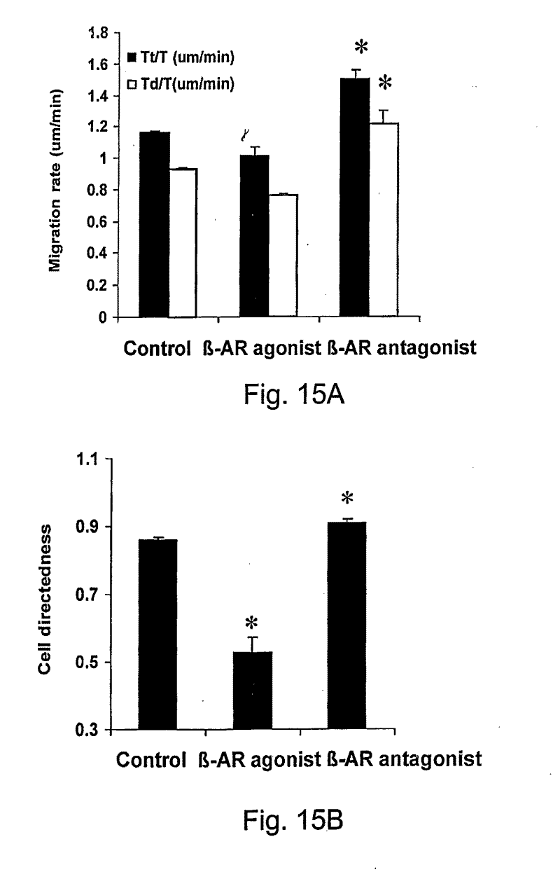

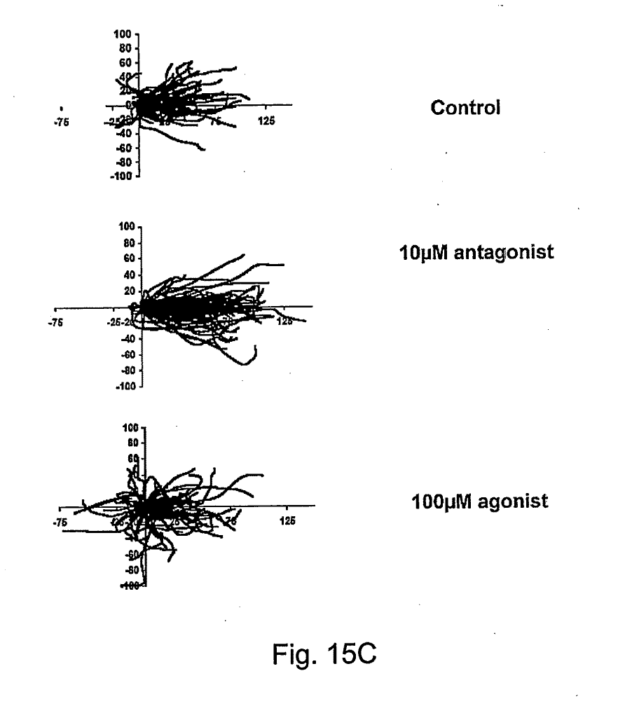

Primary bovine CECs were seeded at low density in electrotactic chambers, the galvanotaxis chamber was assembled, and a DC EF of 50 mV/min was applied as described. Galvanotaxis was performed in the absence or presence of 10 μM β-AR agonist or antagonist. Chambers were placed on a Zeiss Axiovert 100 microscope and the temperature was maintained at 37° C. Time-lapse images were recorded every 5 minutes and analyzed with a MetaMorph system as described. Trajectory speed and displacement speed are represented graphically in FIG. 15 Panel A. Cell directedness is represented graphically in FIG. 15 Panel B. (n=140 (control), n=207 (β-AR agonist), n=185 (β-AR antagonist)). The data shown are representative of multiple independent experiments with cells isolated from numerous bovine corneas. Values plotted are means+/−SEM. * P<0.01 between agonist or antagonist and control.

70 cells in the absence (control) or presence of 10 μM β-AR agonist or 10 β-AR antagonist were tracked over 15 minutes and each cell's trajectory was represented by a black line (FIG. 15 Panel C). A straight horizontal line from left to right represents a cell moving directly towards the cathode with a cosine=1. The length of the line represents the distance traveled by each cell.

CECs migrate cathodally in an applied EF (Farboud et al. (2000) and Zhao et al. (1997), both supra) reminiscent of keratinocytes (Nishimura et al. (1996) “Human keratinocytes migrate to the negative pole in direct current electric fields comparable to those measured in mammalian wounds” J Cell Sci 109:199-207). Here the effect of a β-AR agonist and antagonist on the ability of bovine CECs to sense and respond to an applied EF of 50 mV/mm is observed. Bovine CECs migrate at a rate of 1.17 μM/min with a cosine of 0.86 (FIG. 15 Panels A and B). Upon application of a β-AR agonist, cell speed decreases slightly, but the cosine of cell migration decreases significantly by 39% to 0.53 (FIG. 15 Panels A and B). In contrast, β-AR antagonist treatment significantly increases both the rate of bovine CEC migration by 30% to 1.5 μM/min and the cosine of migration by 6% to 0.91 (FIG. 15 Panels A and B). The vast majority of the β-AR antagonist-treated cells are moving directly to the cathode of the applied EF, demonstrated by the horizontal, parallel trajectories of all the cells in the field of view, in comparison to the more variable trajectories observed for untreated cells and the random trajectories of cells migrating in the presence of β-AR agonist (FIG. 15 Panel C).

β-AR Agonists and Antagonists have No Effect on Adult Human Corneal Epithelial Cell Proliferation

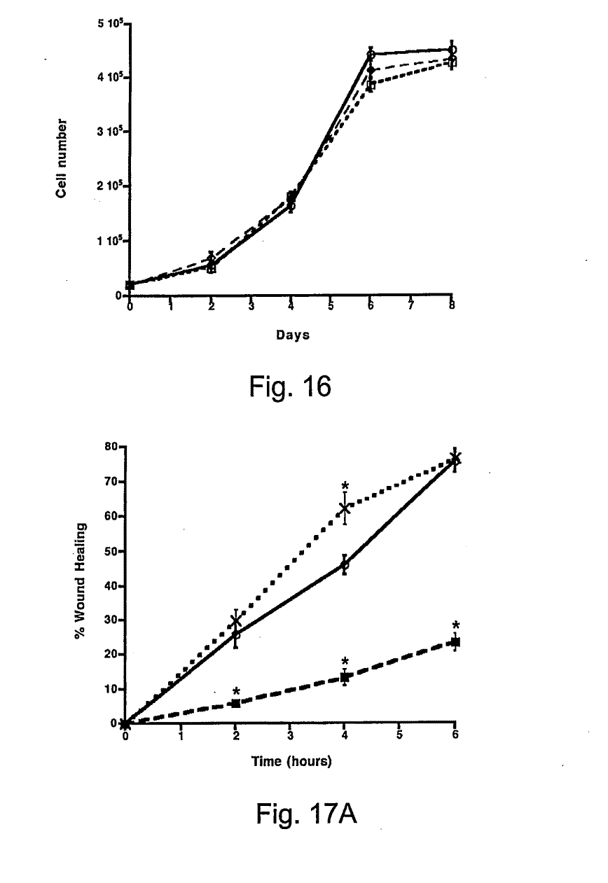

Cell proliferation plays an important role in corneal wound healing (Sharma et al. (2003) “p38 and ERK1/2 coordinate cellular migration and proliferation in epithelial wound healing: evidence of cross-talk activation between MAP kinase cascades” J Biol Chem 278:21989-97). Accordingly, effect of agonist and antagonist treatment on CEC proliferation was examined. 5×10⁴ adult human CECs were plated per well in a 12 well plate in triplicate and allowed to settle and attach to the plate for 2 hours. Cells were incubated in the presence or absence of β-AR agonist (10 nM) or β-AR antagonist (20 μM), (FIG. 16, control (◯), β-AR agonist (□)), β-AR antagonist ( [symbol] )). Cells were harvested and counted on days 2, 4, 6, 8 as described. The data are representative of three independent experiments with at least three different CEC strains. Values plotted are means+/−SEM.

Both β-AR agonists and antagonists have no effect on human CEC proliferation in vitro (FIG. 16). Higher concentrations of β-AR agonist (1 μM) also have no effect on CEC proliferation.

A β-AR Agonist Delays and a β-AR Antagonist Enhances Corneal Epithelial Wound Healing

β2-AR activation on CECs is anti-motogenic (FIGS. 14 and 16), while, in stark contrast, β-AR antagonists are pro-motogenic (FIGS. 12-16). CEC migration is essential for efficient wound re-epithelialization (Zhao et al. (2003) “Direct visualization of a stratified epithelium reveals that wounds heal by unified sliding of cell sheets” Faseb J 17:397-406). To determine if β-AR ligands alter corneal wound healing, the effect of both β-AR agonists and antagonists on bovine corneal wound repair was investigated using whole cornea organ culture, as described.

Bovine eyes were used within a few hours of harvest and a linear wound 200-300 μM wide was created as described. The corneas were surgically removed from the eye using a sterile scalpel blade and immediately transferred to a sterile 6 well tissue culture dish and submerged in 4 ml of CO₂-independent EP media in the presence or absence of 10 μM β-AR agonist or β-AR antagonist. The 6 well dishes were incubated at 37° C. in a humidified atmosphere of 5% CO₂. Corneal wounds were visualized on an inverted Nikon Diaphot 300 microscope using a 20× objective at time 0 and 2, 4 and 6 hours post wounding and images were captured as described at 3 different places along each incisional wound. Image J (a public domain image processing and analysis program developed at the U.S. National Institutes of Health and available on the Internet at rsb.info.nih.gov/ij/) was used to measure the wound area at time 0 and subsequent time points post wounding to calculate the % healing, represented graphically in FIG. 17 Panel A. Significance was taken as *P<0.01, using Student's t test.

Images of one site along representative control, 10 μM β-AR agonist and 10 μM β-AR antagonist-treated wounds at time 0 and 2, 4 and 6 hours post-wounding are presented in FIG. 17 Panel B, with the edge of the wound etched in black for improved clarity.

Bovine corneal wounds in the absence or presence of 10 μM β-AR agonist or 10 μM β-AR antagonist were monitored continually up to 10 hours as described to create movies of the healing process. Photographic images of the control, β-AR agonist and β-AR antagonist-treated wounds at time 0 and 10 hours post-wounding are presented in FIG. 17 Panel C. The arrows on the left mark the original edges of the wound and the arrows on the right mark the edges of each wound at 10 hours post wounding.

Bovine corneal wounds are 75% healed after 6 hours in culture (FIG. 17 Panel A), while a β-AR agonist significantly decreases the rate of healing by 20% after 2 hours, 32.4% after 4 hours and 52.4% after 6 hours. In contrast, a β-AR antagonist significantly increases healing by 16% after 4 hours (FIG. 17 Panel A). Images of corneal wounds, at time 0 and 2, 4 and 6 hours post wounding, highlight the contrasting β-AR agonist-mediated delay and the β-AR antagonist-mediated acceleration of corneal wound repair (FIG. 17 Panel B). Time-lapse images of agonist or antagonist-treated or untreated corneal wounds were recorded every 2 minutes up to 10 hours on a MetaMorph imaging system and the images were compiled into a movie using Quick time software. Additionally, images of corneal wounds immediately after wounding and at the termination of the experiment demonstrate the ability of applied β-AR agonists and antagonists to modulate wound repair up to 10 hours post wounding (FIG. 17 Panel C).

Adult Human Corneal Epithelial Cells Synthesize Epinephrine Endogenously

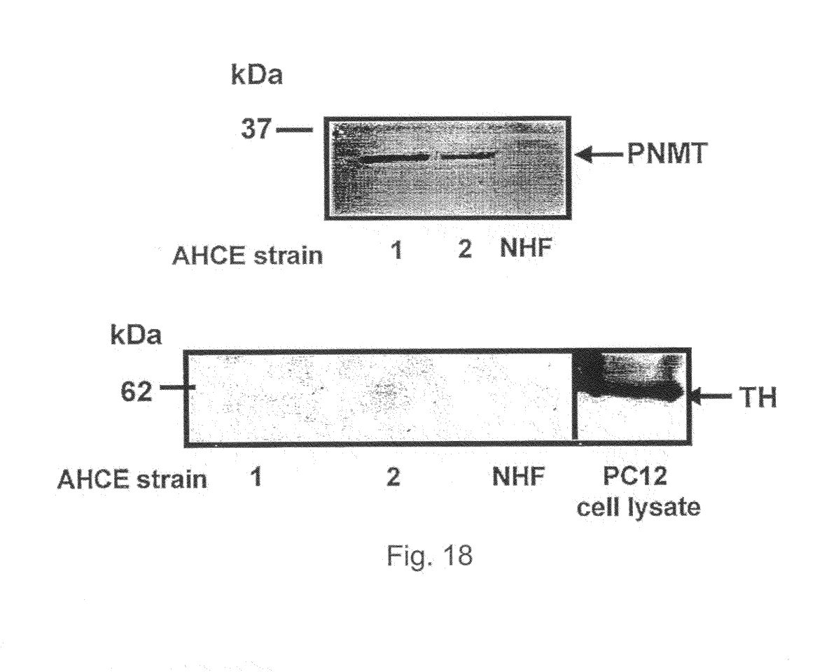

Catecholamines provide important biological functions, acting as both neurotransmitters and endocrine hormones. The conversion of L-tyrosine to L-dopa by tyrosine hydroxylase (TH) is the rate-limiting step for catecholamine biosynthesis and phenylethanolamine-N-methyl transferase (PNMT) catalyzes the synthesis of epinephrine from norepinephrine (FIG. 11 Panel A; Nagatsu et al. (1964a) “Conversion of L-tyrosine to 3,4-dihydroxyphenylalanine by cell-free preparations of brain and sympathetically innervated tissues” Biochem Biophys Res Commun 14:543-9; Nagatsu et al. (1964b) “Tyrosine Hydroxylase The Initial Step in Norepinephrine” Biosynthesis J Biol Chem 239:2910-7; and Schulz et al. (2004) “Principles of catecholamine biosynthesis, metabolism and release” Front Horm Res 31:1-25). To determine if a similar catecholamine synthesis cascade is present in human CECs, cells were lysed and immunoblotted with antibodies specific for TH and PNMT. A PC12 cell lysate was used as a positive control for TH (Nanmoku et al. (2005) “Stimulation of catecholamine biosynthesis via the PKC pathway by prolactin-releasing peptide in PC12 rat pheochromocytoma cells” J Endocrinol 186:233-9) but not for PNMT as PC12 cells contain negligible PNMT (Kano et al. (2005) “Regulatory Roles of Bone Morphogenetic Proteins and Glucocorticoids in Catecholamine Production by Rat Pheochromocytoma Cells” Endocrinology 146:5332-40). A human dermal fibroblast lysate was used as a negative control for both immunoblotting and immunofluorescence (Schallreuter et al. (1992) “Production of catecholamines in the human epidermis” Biochem Biophys Res Commun 189:72-8). Human TH and PNMT enzymes are reported to be around 61-62 kDa and 30-32 kDas in size, respectively (Davidoff et al. (2005) “Catecholamine-synthesizing enzymes in the adult and prenatal human testis” Histochem Cell Biol:1-11). Indeed, the TH and PNMT antibodies detected one major protein at around 61 kDa and 32 kDa, respectively (FIG. 18). The immunoblots of two separate adult human CEC strains are presented in FIG. 18. The data are representative of three independent experiments with three different CEC strains.

To determine the localization of TH and PNMT in human CECs, cell cultures were immunostained with the anti-TH and anti-PNMT antibodies. Multiple brightly stained TH and PNMT-containing circular structures/granules can be observed distributed throughout the cytoplasm of the CECs. Finally, 75 and 146 picograms of epinephrine per milligram protein was measured in extracts from two different strains of human CECs using an epinephrine enzyme immunoassay kit as described, confirming that CECs synthesize epinephrine.

β2-AR is Required for β-Adrenergic Drug-Mediated Modulation of Corneal Epithelial Cell Migration

Transgenic mice in which the β2-AR had been targeted for deletion were obtained as a gift from Dr. B. Kobilka, Md., at Stanford University. Murine corneal epithelial cells were cultured from either male β2-AR +/+ or β2-AR −/− mice, and were treated with mouse corneal growth medium (mCGM), mCGM with 10 nM isoproterenol, or mCGM with 20 μM timolol. Cell migration was monitored microscopically.

Cultured corneal epithelial cells from β2-AR +/+ mice (n=10) treated with a β-AR agonist showed a 70% decrease in migratory speed compared to control cells, while those treated with a β-AR antagonist exhibited a 33% increase in true speed (p<0.001 compared to control). However, when the same treatment groups were repeated using corneal epithelial cells from β2-AR −/− mice (n=10), all treatment groups showed statistically equivalent migratory speeds (p>0.05), demonstrating dependence on expression of the β2-AR.

β-Adrenergic Agents Modulate Corneal Wound Healing In Vivo

Using male β2-AR +/+ or β2-AR −/− mice, 2 mm diameter circular corneal epithelial wounds (OD) were created using a crescent blade. The corneas were treated topically with Balanced Salt Solution (BSS), BSS with 1% isoproterenol, or BSS with 0.5% timolol, and the wound healing was monitored stereomicroscopically using fluorescein.

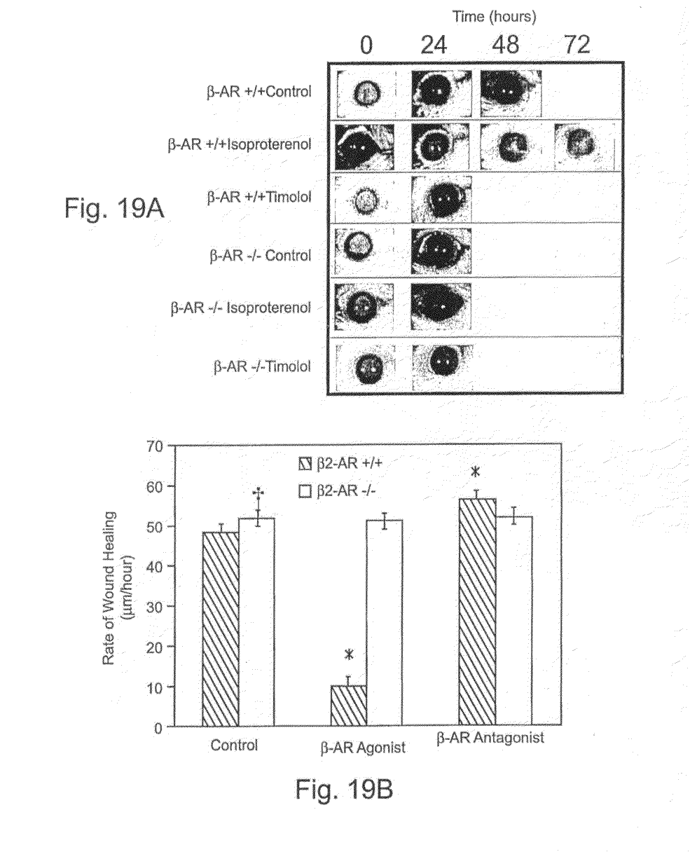

Photographs showing healing over time of fluorescein stained corneal epithelial wounds from β2-AR +/+ (n=24) and −/− mice (n=24) treated with BSS (control), isoproterenol (agonist), or timolol (antagonist) are presented in FIG. 19 Panel A. Using regression analysis of the linear phase of wound healing and the F-test, the rates of wound healing showed that a β-AR agonist delays wound healing by nearly 80% in β2-AR +/+ mice but not in β2-AR −/− mice; see FIG. 19 Panel B. Conversely, a β-AR antagonist increased the rate of healing significantly in β2-AR +/+ mice but not in β2-AR −/− mice, showing the isoproterenol and timolol modulation of wound healing is via the β2-AR. *p<0.05. Lastly, in the control group β2-AR −/− mice healed at a faster rate than β2-AR +/+mice. †p<0.06.

β-AR-Mediated Modulation of Corneal Epithelial Wound Repair

This example demonstrates that the β2-AR can modulate CEC migration, galvanotaxis and corneal wound healing. A β-AR agonist decreases the rate of human CEC migration and alters cytoskeletal conformation via a PP2A-dependent mechanism, partially blinds bovine CECs to an applied EF, and delays bovine corneal epithelial wound healing. In contrast, a β-AR antagonist increases human CEC migration, increases ERK phosphorylation, enhances the ability of bovine CECs to sense and respond to an applied EF, and accelerates bovine corneal epithelial wound healing. The example also presents the novel finding that CECs endogenously synthesize epinephrine. Without intending to be limited to any particular mechanism, the mechanism for the β-AR agonist-mediated promotion of corneal wound healing can be via β2-AR blockade, preventing the endogenously synthesized β-AR agonist from exerting its anti-motogenic effects.

In vitro, β2-AR activation decreases the rate of corneal epithelial single cell migration and remodels the CEC cytoskeleton from that of an actively migrating cell to that of a static adherent one, with a dense network of cortical actin fibers just beneath the plasma membrane and focal adhesions distributed evenly around the cell periphery. The β-AR agonist-mediated alterations in cell migration and cytoskeletal morphology are reversed when the cells are pre-treated with the membrane permeant phosphatase inhibitor, okadaic acid, at a concentration highly selective for PP2A (100 nM) (Namboodiripad and Jennings (1996) “Permeability characteristics of erythrocyte membrane to okadaic acid and calyculin” A Am J Physiol 270:C449-56; Bialojan and Takai (1988) supra; Millward et al. (1999) supra). The β-AR agonist-mediated anti-motogenic effects are, therefore, generated via a PP2A-dependent mechanism. Wound repair is dependent on the temporally and spatially controlled reorganization of the actin cytoskeleton to allow efficient cell migration (Pantaloni et al. (2001) supra). Integrin receptors become relocalized in the wounded cornea (Stepp et al. (1993) “Integrins in the wounded and unwounded stratified squamous epithelium of the cornea” Invest Opthalmol Vis Sci 34:1829-44 and Latvala et al. (1996) “Distribution of alpha 6 and beta 4 integrins following epithelial abrasion in the rabbit cornea” Acta Opthalmol Scand 74:21-5) allowing the migrating epithelial cells to interact with the variety of extra cellular matrices found in the wound site (Larjava et al. (1993) “Expression of integrins and basement membrane components by wound keratinocytes” J Clin Invest 92:1425-35). β1 integrin is also a substrate for PP2A (Mulrooney et al. (2000) “Phosphorylation of the beta1 integrin cytoplasmic domain: toward an understanding of function and mechanism” Exp Cell Res 258:332-41) as well as numerous other proteins that either reside in FAs or play a role in migration including, but not limited to FAK, paxillin, Akt and she (Kawada et al. (1999) “Cytostatin, an inhibitor of cell adhesion to extracellular matrix, selectively inhibits protein phosphatase 2A” Biochim Biophys. Acta 1452:209-17; Ugi et al. (2004) “Protein phosphatase 2A negatively regulates insulin's metabolic signaling pathway by inhibiting Akt (protein kinase B) activity in 3T3-L1 adipocytes” Mol Cell Biol 24:8778-89; and Kiely et al. (2005) “RACK1-mediated integration of adhesion and insulin-like growth factor I (IGF-I) signaling and cell migration are defective in cells expressing an IGF-I receptor mutated at tyrosines 1250 and 1251” J Biol Chem 280:7624-33). The β-AR agonist-mediated PP2A activation described here optionally also alters numerous other migration and adhesion pathways.

In contrast to the anti-motogenic effects of the β-AR agonist, β-AR antagonists accelerate the healing of scratch wounds, increase the rate of single cell migration, and increase ERK phosphorylation, while maintaining the cytoskeletal conformation of an actively migrating cell.

Wound currents have been measured exiting injured cornea (Chiang et al. (1992) “Electrical fields in the vicinity of epithelial wounds in the isolated bovine eye” Exp Eye Res 54:999-1003) and play a role in corneal wound healing (Reid et al. (2005) supra) and limb regeneration in salamanders and newts (Altizer et al. (2002) “Skin flaps inhibit both the current of injury at the amputation surface and regeneration of that limb in newts” J Exp Zool 293:467-77 and Borgens et al. (1984) “Stump currents in regenerating salamanders and newts” J Exp Zool 231:249-56). EF application also influences cell division (Zhao et al. (1999) “Small, physiological electric field orients cell division” Proc Natl Acad Sci USA 96:4942-6) and migration by initiating galvanotaxis within minutes (Farboud et al. (2000) and Zhao et al. (1997), both supra). Indeed, as the EF is generated immediately upon wounding, it may be the earliest signal that epithelial cells receive to initiate and guide CEC migration into the wound bed. Previously, it has been demonstrated that PKA (Pullar et al. (2001) “Cyclic AMP-dependent protein kinase A plays a role in the directed migration of human keratinocytes in a DC electric field” Cell Motil Cytoskeleton 50:207-17) and the β2-AR-mediated increase in intracellular cAMP (Pullar and Isseroff (2005b) supra) can modulate keratinocyte galvanotaxis. The experiments described herein demonstrate that β-AR activation can also modulate the ability of bovine CECs to sense and respond to an applied EF. While the β-AR agonist partially blinds the bovine CECs to the applied EF, the antagonist appears to increase the ability of the cells to sense and respond to the EF by exhibiting enhanced directionality and rate of migration, reminiscent of its effect on keratinocyte galvanotaxis (Pullar and Isseroff (2005b) supra). The β-AR-mediated modulation of EF-directed bovine CEC migration is optionally dependent on a cAMP-dependent signaling cascade, as reported in keratinocytes (Pullar and Isseroff (2005b) and Pullar et al. (2001), both supra). The ability of the β-AR to modulate epithelial cell galvanotaxis suggests that a potential interaction/co-operation may exist between endogenous EFs, known to be important guidance cues in development and wound healing (McCaig et al. (2005) “Controlling cell behavior electrically: current views and future potential” Physiol Rev 85:943-78 and Robinson (1985) “The responses of cells to electrical fields: a review” J Cell Biol 101:2023-7), and the epithelial adrenergic networks.

The significant delay in the healing of β-AR agonist-treated and acceleration in the healing of β-AR antagonist-treated bovine and murine corneal wounds provides strong evidence for the role of β2-AR in wound repair. Using murine or bovine corneal tissue confers the advantages of a physiological extra cellular matrix and the three dimensional geometry of the healing wound, not found in scratch assays or other assays using cultured cells. Thus this work documents specific β-AR-mediated changes in CEC biology with the resultant alteration in the rate of corneal wound healing. In addition, the murine experiments are performed in vivo, and confer the advantage of an intact animal in which to examine the effects of the beta agonists and antagoists. It is thus very strong confirmatory evidence that these agents impair (agonists) and improve (antagonists) healing of the corneal epithelium in vivo in the intact animal.

Catecholamines have been detected in lacrimal secretions from healthy volunteers (Trope and Rumley (1984) and Zubareva and Kiseleva (1977), both supra), but their presence has been attributed to the sympathetic nerves that terminate in the cornea (Rozsa and Beuerman (1982) “Density and organization of free nerve endings in the corneal epithelium of the rabbit” Pain 14:105-20 and Toivanen et al. (1987) “Histochemical demonstration of adrenergic nerves in the stroma of human cornea” Invest Opthalmol Vis Sci 28:398-400). However, β-AR-antagonist-mediated pro-motogenic effects on CECs are observed in culture, in the absence of sympathetic innervation, suggesting the presence of an adrenergic hormonal mediator network in the CECs themselves. Indeed, critical catecholamine synthesis enzymes are detected in CEC lysates, localized to granules or vesicles in the cytoplasm, and epinephrine can be measured in CEC extracts, demonstrating that CECs synthesize epinephrine endogenously.

The identification of an endogenous catecholamine synthesis network in CECs adds a new dimension and level of complexity to the adrenergic network in the cornea and the role it can play in controlling corneal homeostasis and wound healing. Whereas the presence of catecholamines in the eye has previously been attributed to corneal sympathetic nerves or their stress-induced release from the adrenal medulla (Garcia-Hirschfeld et al. (1994) “Neurotrophic influences on corneal epithelial cells” Exp Eye Res 59:597-605; Jones and Marfurt (1996) “Sympathetic stimulation of corneal epithelial proliferation in wounded and nonwounded rat eyes” Invest Opthalmol Vis Sci 37:2535-47; Marfurt and Ellis (1993)“Immunohistochemical localization of tyrosine hydroxylase in corneal nerves” J Comp Neurol 336:517-31; Perez et al. (1987) “Effects of chronic sympathetic stimulation on corneal wound healing” Invest Opthalmol Vis Sci 28:221-4; Yoshitomi and Gregory (1991) “Ocular adrenergic nerves contribute to control of the circadian rhythm of aqueous flow in rabbits” Invest Opthalmol Vis Sci 32:523-8; Detillion et al. (2004) “Social facilitation of wound healing” Psychoneuroendocrinology 29:1004-11; and Nankova and Sabban (1999) “Multiple signaling pathways exist in the stress-triggered regulation of gene expression for catecholamine biosynthetic enzymes and several neuropeptides in the rat adrenal medulla” Acta Physiol Scand 167:1-9), the experiments described herein demonstrate that CECs themselves are a source of catecholamines, generating a local endogenous β-adrenergic network within the corneal epithelium.

This finding is novel and can have far-reaching implications for the medical field due to the prolific use of β-AR agonists and antagonists in ophthalmic, cardiac and pulmonary medicine. Nearly 50 million Americans are treated daily with β-AR antagonists, more commonly known as β-blockers (North Suburban Cardiology Group, Ltd. report (2001) “Facts About Hypertension”), and an estimated 14.2 million Americans are treated with β-AR agonists for asthma (Asthma and Allergy Foundation report (2000) “Costs of Asthma in America”). β-AR ligands are also widely used in ocular medicine. β-AR antagonists are the most frequently prescribed class of drug for the treatment of glaucoma, a disease estimated to affect 1.25% of the population over 40 years of age and the leading cause of irreversible blindness in the world (Medeiros and Weinreb (2002). “Medical backgrounders: glaucoma” Drugs Today (Bare) 38:563-70). The World Health Organization reported that 5.1 million people were bilaterally blinded from glaucoma in 1995 (Coleman and Brigatti (2001) “The glaucomas” Minerva Med 92:365-79). Elevated intraocular pressure is a major risk factor associated with glaucoma (Quigley (1996) “Number of people with glaucoma worldwide” Br J Opthalmol 80:389-93) and β-AR antagonists are prescribed to lower it, therefore minimizing damage to the optic nerve (Zimmerman (1993) “Topical ophthalmic beta blockers: a comparative review” J Ocul Pharmacol 9:373-84). Additionally, epinephrine is widely used by ophthalmologists to maintain mydriasis during cataract surgery (Corbett and Richards (1994) “Intraocular adrenaline maintains mydriasis during cataract surgery” Br J Opthalmol 78:95-8). The results herein demonstrate exogenous β-AR ligands modulate both CECs and corneal wound healing, providing mechanistic support for the regulatory role of the β-adrenergic hormonal network in the corneal wound repair process and providing novel therapies for modulating wound healing.

### Example 4

### β-Adrenergic Receptor Agonists Delay while Antagonists Accelerate Burn Wound Healing

The following sets forth a series of experiments that demonstrate use of β2-AR agonists and antagonists to modulate the rate of re-epithelialization of burn wounds in human skin explants.

Since burn wounded patients have elevated levels of the circulating β2-AR activator epinephrine, whether blockade of β2-ARs increases keratinocyte migration and has the potential to accelerate burn wound re-epithelialization was examined. When cultured human keratinocytes were incubated with epinephrine at levels equivalent to those measured post-burn (10 nM), their migratory speed decreased by 75%. Addition of the β2-AR blocker timolol (10 μM) restored the migratory speed to control levels. When confluent cultures of human keratinocytes were scratch wounded, epinephrine pretreatment reduced healing by 70% and the addition of timolol reversed the effects of epinephrine and accelerated healing by 20%. Western blot analysis of scratch wounded confluent keratinocyte cultures showed downregulation of the β2-AR one hour after the scratch, and addition of timolol delayed this β2-AR downregulation.

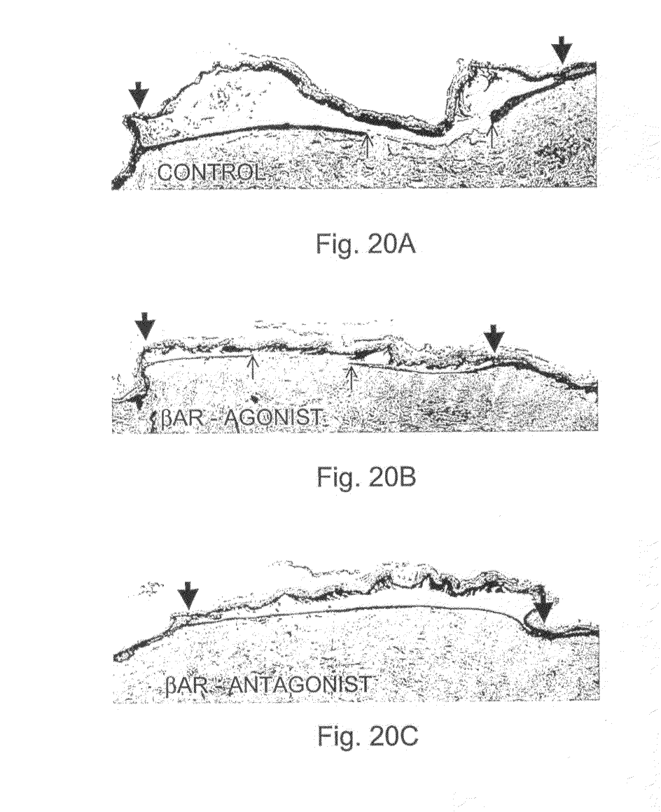

An ex vivo model of burn wound healing was developed. A heated brass rod was applied to excised human skin, obtained and maintained basically as described above in Example 1. Burn wounds were treated with β-AR agonist (10 μm isoproterenol) or β-AR antagonist (10 μm timolol). As shown in FIG. 20, at day ten after the burn wound, the control (Panel A) and β-AR agonist treated (Panel B) wounds did not fully re-epithelialize, but the β-AR antagonist treated (Panel C) wounds fully re-epithelialized. Thick arrows represent the burn wound edge and thin arrows represent the edge of the re-epithelialization from the wound edge. Images were taken at 40× magnification. At day ten after the burn, re-epithelialization was delayed 14% when treated with β2-AR activator (isoproterenol), and was conversely enhanced 37% when treated with antagonist (timolol).

These results demonstrated that the burn wound adrenergic environment significantly decreased keratinocyte migration and delayed burn wound re-epithelialization. Furthermore, the addition of the β2-AR blocker timolol significantly enhanced keratinocyte migration and accelerated burn wound re-epithelialization, indicating that antagonist treatment provides novel therapies for modulating burn wound healing.

While the foregoing invention has been described in some detail for purposes of clarity and understanding, it will be clear to one skilled in the art from a reading of this disclosure that various changes in form and detail can be made without departing from the true scope of the invention. For example, all the techniques and compositions described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

1. A pharmaceutical composition comprising a beta-2 adrenergic receptor antagonist, wherein the composition is formulated for topical delivery of the antagonist to a tissue or organ other than an eye.

2-7. (canceled)

8. A pharmaceutical composition comprising a beta-2 adrenergic receptor agonist, wherein the composition is formulated for topical delivery of the agonist to a tissue or organ other than an eye or a tissue or organ comprising a respiratory tract.

9-12. (canceled)

13. A kit comprising:
a pharmaceutical composition comprising a beta-2 adrenergic receptor agonist or antagonist; and
instructions for administering the composition to a patient comprising or at risk for comprising a wound in an epithelial tissue;
packaged in one or more containers.

14-22. (canceled)

23. A method for increasing a rate of wound healing in a target patient, the method comprising:
identifying the target patient by identifying a person comprising or at risk for comprising a wound in an epithelial tissue; and
topically administering an effective amount of a beta-2 adrenergic receptor antagonist to the target patient.

24. The method of claim 23, wherein the wound comprises a chronic skin wound.

25. The method of claim 23, wherein the wound comprises a venous stasis ulcer, a diabetic foot ulcer, a neuropathic ulcer, or a decubitus ulcer.

26. The method of claim 23, wherein the wound comprises a wound resulting from surgical wound dehiscence.

27. The method of claim 23, wherein the wound comprises a burn.

28. The method of claim 23, wherein the epithelial tissue comprises skin.

29. The method of claim 23, wherein the epithelial tissue comprises a genitourinary epithelium, a gastrointestinal epithelium, or a pulmonary epithelium.

30. The method of claim 23, wherein the epithelial tissue comprises a corneal epithelium.

31. The method of claim 23, wherein the antagonist is topically administered by application of an ointment, cream, lotion, gel, suspension, or spray comprising the antagonist to the wound.

32. The method of claim 23, wherein the antagonist is topically administered by application of a dressing comprising the antagonist to the wound.

33. The method of claim 23, wherein the antagonist is topically administered by introduction of a foam comprising the antagonist to an epithelial-lined cavity comprising the wound.

34. The method of claim 23, wherein the antagonist is administered after the wound is created.

35. The method of claim 23, wherein the antagonist is selected from the group consisting of: timolol, labetalol, dilevelol, propanolol, carvedilol, nadolol, carteolol, penbutolol, sotalol, ICI 118,551, and butoxamine.

36. The method of claim 23, wherein the antagonist has a K_{d} for a beta-3 adrenergic receptor that is about 100 or more times greater than a K_{d} of the antagonist for a beta-2 adrenergic receptor.

37. The method of claim 23, wherein the antagonist is substantially free of activity as a beta-3 adrenergic receptor agonist.

38. A method for increasing a rate of wound healing in a target patient, the method comprising:
identifying the target patient by identifying a person comprising or at risk for comprising a wound in an epithelial tissue, wherein the wound is other than a burn; and
administering an effective amount of a beta-2 adrenergic receptor antagonist to the target patient.

39-55. (canceled)

56. A method for increasing a rate of wound healing in a target patient, the method comprising:
identifying the target patient by identifying a person comprising or at risk for comprising a wound in an epithelial tissue; and
administering an effective amount of a beta-2 adrenergic receptor antagonist to the target patient, wherein the rate of wound healing is at least about 10% greater than in a corresponding untreated individual.

57-75. (canceled)

76. A method for increasing a rate of wound healing in a target patient, the method comprising:
identifying the target patient by identifying a person comprising or at risk for comprising a wound in an epithelial tissue;
wherein the wound is a burn covering less than about 40% of the patient's total body surface area, or wherein the wound is a burn and the patient does not display hypermetabolic syndrome; and
administering an effective amount of a beta-2 adrenergic receptor antagonist to the target patient.

77-84. (canceled)

85. A method for decreasing cell growth around a device implanted in a target organism, the method comprising:
identifying the target organism by identifying an organism having or expected to have a device implanted in the organism; and
administering to the target organism an effective amount of a beta-2 adrenergic receptor agonist.

86-91. (canceled)

92. A coated device for implantation in an organism, comprising:
a device, and
a coating on a surface of the device, the coating comprising a beta-2 adrenergic receptor agonist.

93-94. (canceled)

95. A method for decreasing wound contraction in a target patient, the method comprising:
identifying the target patient by identifying a person comprising or at risk for comprising a wound in an epithelial tissue; and
administering an effective amount of a beta-2 adrenergic receptor agonist to the target patient.

96-103. (canceled)
